(19) United States
(12) Patent Application Publication
(10) Pub. No.: US 2026/0274865 A1
(43) Pub. Date: Sep. 17, 2026

(54) Fused Ring Compound and Preparation Method Therefor and Use Thereof

(71) Applicant: SICHUAN KELUN-BIOTECH BIOPHARMACEUTICAL CO., LTD., Chengdu, Sichuan (CN)

(72) Inventors: Qiang Tian, Chengdu, Sichuan (CN); Yitao Zhang, Chengdu, Sichuan (CN); Jian Ye, Chengdu, Sichuan (CN); Wanli Liu, Chengdu, Sichuan (CN); Yile Li, Chengdu, Sichuan (CN); Yu Yang, Chengdu, Sichuan (CN); Xiaoxi Yuan, Chengdu, Sichuan (CN); Hongmei Song, Chengdu, Sichuan (CN); Junyou Ge, Chengdu, Sichuan (CN)

(73) Assignee: SICHUAN KELUN-BIOTECH BIOPHARMACEUTICAL CO., LTD., Chengdu, Sichuan (CN)

(21) Appl. No.: 19/469,673

(22) PCT Filed: May 10, 2024
(86) PCT No.: PCT/CN2024/092337

§ 371 Date: Sep. 26, 2025

(30) Foreign Application Priority Data
May 12, 2023 (CN) 202310538792.7

(51) Int. Cl. C07D 519/00; A61K 31/5517; A61K 47/68; A61P 35/00; C07B 59/00

(52) U.S. Cl. CPC C07D 519/00; A61K 31/5517; A61K 47/6803; A61P 35/00; C07B 59/002

(57) ABSTRACT

A fused ring compound and a preparation method therefor and a use thereof. The compound has excellent therapeutic effect on solid tumors such as gastric cancer, breast cancer, lung cancer or urothelial cancer, or hematological malignancies.

The present application is based on and claims priority to the Chinese application with application number CN 202310538792.7 and application date May 12, 2023, the content of which is hereby incorporated by reference in its entirety.

### TECHNICAL FIELD

The present application relates to the field of pharmaceuticals, and specifically to a fused-ring compound and preparation method therefor and use thereof.

### BACKGROUND ART

Antibody-drug conjugate (ADC) is typically composed of a monoclonal antibody, a bioactive molecule (primarily a tumor-killing cytotoxin) and a linker. The bioactive molecule is covalently coupled to the antibody via the linker. The antibody recognizes a specific target on the surface of tumor cells, directing the ADC to the tumor microenvironment and the surface of cancer cell. Through endocytosis, the ADC enters the cancer cells, where the bioactive molecule is released. By inhibiting the tubulin of the cancer cells or damaging the DNA of the cancer cells, the bioactive molecule kills the cancer cells while minimizing damage to normal tissue cells.

The bioactive molecules suitable for ADCs mainly include anti-tubulin agents, DNA intercalators, DNA topoisomerase inhibitors, RNA polymerase inhibitors and gene transcription inhibitors. At present, the payloads used in commercially available ADCs as well as those in clinical trials are predominantly microtubule inhibitors, such as MMAE, MMAF, DM1 and DM4.

The objective of the present invention is to provide a novel fused-ring compound with excellent anti-tumor activity and is suitable for the preparation of antibody-drug conjugates (ADCs). The conjugates have good stability, homogeneity, hydrophilicity, efficacy and safety.

### CONTENTS OF THE PRESENT INVENTION

### Compound

The first aspect of the present invention provides a compound or a pharmaceutically acceptable salt, ester, stereoisomer, polymorph, solvate, N-oxide, isotopically-labeled substance, metabolite or prodrug thereof, wherein the compound has a structure represented by the following formula:

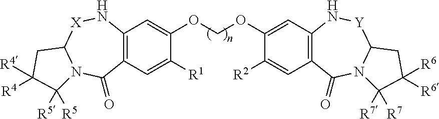

wherein, R¹ and R² are each independently selected from the group consisting of hydrogen, —CN, halogen, —OR^{a}, —NH₂, —NH(C₁₋₆ alkyl), —N(C₁₋₆ alkyl)₂, C₁₋₆ alkyl, C₂₋₆ alkenyl, C₂₋₆ alkynyl, C₃₋₆ cycloalkyl, 3- to 6-membered heterocyclyl, C₆₋₁₀ aryl and 5- to 10-membered heteroaryl; the alkyl, alkenyl, alkynyl, cycloalkyl, heterocyclyl, aryl and heteroaryl are optionally substituted with one or more substituents selected from the group consisting of —CN, halogen, —OH, —NH₂, C₁₋₆ alkyl, C₁₋₆ alkoxy, C₂₋₆ alkenyl, C₂₋₆ alkynyl, C₃₋₆ cycloalkyl, 3- to 6-membered heterocyclyl, C₆₋₁₀ aryl and 5- to 10-membered heteroaryl;
R⁴ and R^{4′} are each independently selected from the group consisting of hydrogen, halogen, —OH, —NH₂, C₁₋₆ alkyl, halogenated C₁₋₆ alkyl and C₁₋₆ alkoxy, and both R⁵ and R^{5′} are hydrogen; or, R⁴ and R^{4′}, together with the carbon atom connecting R⁴ and R^{4′}, form a carbon-carbon double bond or a 3- to 6-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3- to 6-membered carbocyclic ring, and R^{5′} and R^{4′} are each independently selected from the group consisting of hydrogen, halogen, —NH₂, C₁₋₆ alkyl, halogenated C₁₋₆ alkyl and C₁₋₆ alkoxy;
R⁶ is hydrogen, C₁₋₆ alkyl or

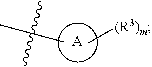

R^{6′}, R⁷ and R^{7′} are all hydrogen; or, R^{6′} and R⁷ are connected to form a bond, and R^{7′} is hydrogen;
R^{a} is selected from the group consisting of H, C₁₋₆ alkyl or C₃₋₆ cycloalkyl;
Ring A is selected from the group consisting of 3- to 6-membered cycloalkyl, C₆₋₁₀ aryl, 5- to 10-membered heteroaryl, 3- to 6-membered heterocyclyl;
R³ is selected from the group consisting of hydrogen, —CN, halogen, —OR^{a}, —NH₂, —NH(C₁₋₆ alkyl), —N(C₁₋₆ alkyl)₂, C₁₋₆ alkyl, C₃₋₆ cycloalkyl, 3- to 6-membered heterocyclyl, C₆₋₁₀ aryl and 5- to 10-membered heteroaryl; the alkyl, alkenyl, alkynyl, cycloalkyl, heterocyclyl, aryl and heteroaryl are optionally substituted with one or more substituents selected from the group consisting of —CN, halogen, —OH, —NH₂, C₁₋₆ alkyl, C₁₋₆ alkoxy, C₃₋₆ cycloalkyl, 3- to 6-membered heterocyclyl, C₆₋₁₀ aryl and 5- to 10-membered heteroaryl;
X and Y are each independently selected from the group consisting of —CH₂—, —CD₂-,

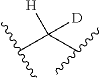 [SMILES: *C(*)[2H]]

and —C(O)—;
m is selected from 1 to 5, such as 1, 2, 3, 4 or 5;
n is selected from 1 to 10, such as 1, 2, 3, 4, 5, 6, 7, 8, 9 or 10;
when n is 5 and Ring A is phenyl, R³ is not methoxy.

In some embodiments, R¹ is selected from the group consisting of hydrogen, —CN, halogen, —OR^{a} and C₁₋₆ alkyl, wherein R^{a} is selected from the group consisting of C₁₋₆ alkyl, deuterated C₁₋₆ alkyl and C₃₋₆ cycloalkyl.

In some embodiments, R¹ is selected from the group consisting of —OR^{a}, wherein R^{a} is selected from the group consisting of C₁₋₆ alkyl, deuterated C₁₋₆ alkyl and C₃₋₆ cycloalkyl.

In some embodiments, R¹ is selected from the group consisting of methoxy, trideuterated methoxy and —O-cyclopropyl.

In some embodiments, R¹ is selected from the group consisting of hydrogen, —CN, halogen, —OR^{a} and C₁₋₆ alkyl, wherein R^{a} is C₁₋₆ alkyl or C₃₋₆ cycloalkyl.

In some embodiments, R¹ is —OR^{a}, wherein R^{a} is C₁₋₆ alkyl or C₃₋₆ cycloalkyl.

In some embodiments, R¹ is methoxy or —O-cyclopropyl.

In some embodiments, R² is selected from the group consisting of hydrogen, —CN, halogen, —OR^{a} and C₁₋₆ alkyl, wherein R^{a} is C₁₋₆ alkyl or C₃₋₆ cycloalkyl.

In some embodiments, R² is —OR^{a}, wherein R^{a} is C₁₋₆ alkyl.

In some embodiments, R² is methoxy.

In some embodiments, R⁴ is hydrogen or halogen, R^{4′} is selected from the group consisting of hydrogen, halogen, —OH, —NH₂, C₁₋₆ alkyl, halogenated C₁₋₆ alkyl and C₁₋₆ alkoxy, both R⁵ and R^{5′} are hydrogen; or, R⁴ and R^{4′}, together with the carbon atom connecting R⁴ and R^{4′}, form a carbon-carbon double bond or a 3- to 6-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3- to 6-membered carbocyclic ring, and both R^{5′} and R^{4′} are hydrogen.

In some embodiments, R⁴ is hydrogen or fluorine, R^{4′} is selected from the group consisting of hydrogen, fluorine, —OH, —NH₂, methyl, trifluoromethyl and methoxy, both R⁵ and R^{5′} are hydrogen; or, R⁴ and R^{4′}, together with the carbon atom connecting R⁴ and R^{4′}, form a carbon-carbon double bond or a 3- to 6-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3-membered carbocyclic ring, and both R^{5′} and R^{4′} are hydrogen.

In some embodiments, R⁴ is hydrogen or fluorine, R^{4′} is selected from the group consisting of hydrogen, fluorine, —OH, —NH₂, methyl, trifluoromethyl and methoxy, and both R⁵ and R^{5′} are hydrogen; or, R⁴ and R^{4′}, together with the carbon atom connecting R⁴ and R^{4′}, form a carbon-carbon double bond, and both R⁵ and R^{5′} are hydrogen; or, R⁴ and R⁵ are connected to form a bond, or R⁴ and R⁵, together with the carbon atom connecting R⁴ and R⁵, form a 3-membered carbocyclic ring, and both R^{5′} and R^{4′} are hydrogen.

In some embodiments, R⁴ and R^{4′}, together with the carbon atom connecting R⁴ and R^{4′}, form a 3-membered carbocyclic ring, and both R⁵ and R^{5′} are hydrogen.

In some embodiments, R⁶ is

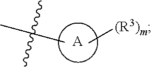

Ring A is selected from the group consisting of 3- to 6-membered cycloalkyl, C₆₋₁₀ aryl or 5- to 10-membered heteroaryl;
R³ is selected from the group consisting of hydrogen, —CN, halogen, —OR^{a}, —NH₂, —NH(C₁₋₆ alkyl), —N(C₁₋₆ alkyl)₂, C₁₋₆ alkyl, C₃₋₆ cycloalkyl, 3- to 6-membered heterocyclyl, C₆₋₁₀ aryl and 5- to 10-membered heteroaryl; the alkyl, alkenyl, alkynyl, cycloalkyl, heterocyclyl, aryl and heteroaryl are optionally substituted with one or more substituents selected from the group consisting of —CN, halogen, —OH, —NH₂, C₁₋₆ alkyl, C₁₋₆ alkoxy, C₃₋₆ cycloalkyl, 3- to 6-membered heterocyclyl, C₆₋₁₀ aryl and 5- to 10-membered heteroaryl;
R^{a} is selected from the group consisting of H and C₁₋₆ alkyl;
m is selected from 1, 2, 3, 4 and 5.

In some embodiments, R⁶ is

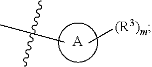

Ring A is selected from the group consisting of cyclopropyl, phenyl, furanyl, thienyl, pyrimidinyl, and pyridinyl;
R³ is selected from the group consisting of hydrogen, fluorine, —OH, methoxy, —NH₂, and hydroxymethyl;
m is 1 or 2.

In some embodiments, R⁶ is

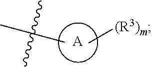

Ring A is phenyl;
R³ is selected from the group consisting of hydrogen, fluorine, —OH, methoxy, —NH₂, and hydroxymethyl;
m is 1 or 2.

In some embodiments, Ring A is C₆₋₁₀ aryl or 5- to 10-membered heteroaryl.

In some embodiments, Ring A is selected from the group consisting of phenyl, pyrimidinyl, pyridinyl, furanyl, and thienyl.

In some embodiments, R³ is selected from the group consisting of —OH, methoxy, —NH₂, and hydroxymethyl.

In some embodiments, m is 1 or 2.

In some embodiments, m is 1.

In some embodiments, R⁶ is selected from the group consisting of phenyl, pyridinyl,

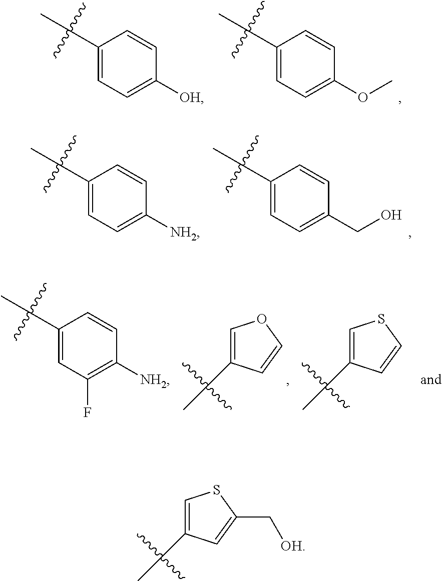

In some embodiments, R⁶ is selected from the group consisting of phenyl, pyridinyl,

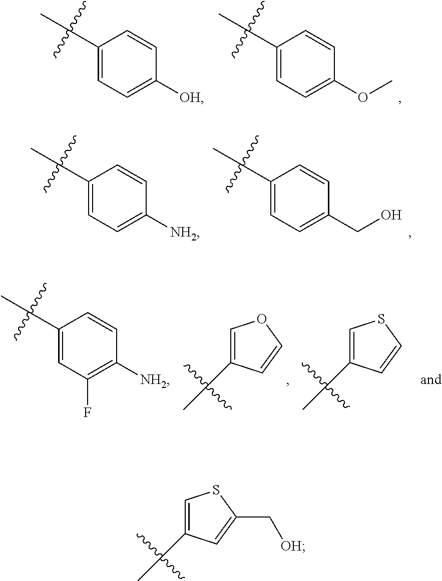

R^{6′} is bonded to R⁷, and R^{7′} is hydrogen.

In some embodiments, R⁶ is

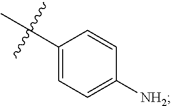 [SMILES: *c1ccc(N)cc1]

R^{6′}, R⁷ and R^{7′} are all hydrogen.

In some embodiments, n is 3 to 8, such as 3, 4, 5, 6, 7 or 8.

In some embodiments, X is —CH₂— or —CD₂-; optionally, Y is selected from the group consisting of —CH₂—, —CD₂-,

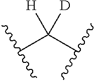 [SMILES: *C(*)[2H]]

### and —C(O)—.

In some embodiments, X is —CH₂—, Y is —CH₂—; X is —CH₂—, Y is —CD₂- or —C(O)—; or X is —CD₂-, Y is —CH₂—.

In some embodiments, X is —CH₂—, Y is —CH₂—.

In some embodiments, the compound has a structure represented by Formula (I-1) or Formula (I-2):

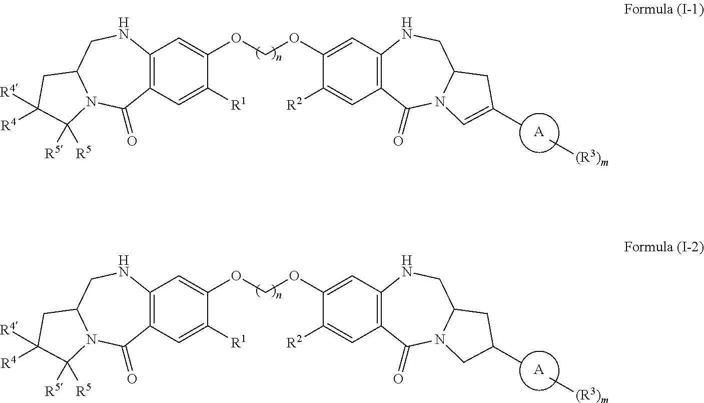

wherein, R¹, R², R³, R⁴, R^{4′}, R⁵, R^{5′}, Ring A, m and n are as described above.

In some embodiments, the compound has a structure represented by Formula (II-1) or Formula (II-2):

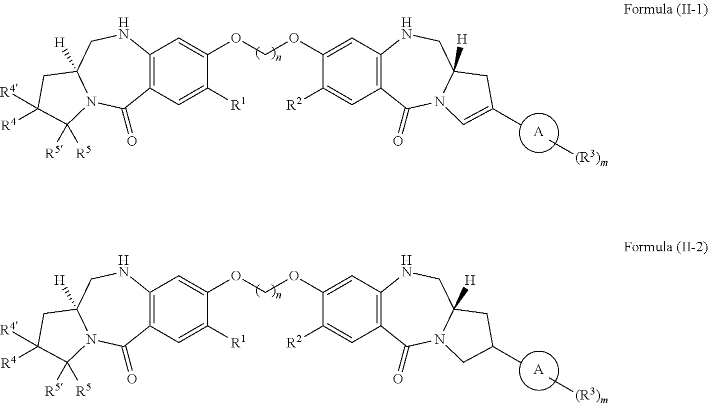

wherein, R¹, R², R³, R⁴, R^{4′}, R⁵, R^{5′}, Ring A, m and n are as described above.

In some embodiments, in any of the above general formulas,
R¹ is selected from the group consisting of methoxy, trideuterated methoxy and —O-cyclopropyl;
R² is methoxy;
R⁴ is hydrogen or fluorine, R^{4′} is selected from the group consisting of hydrogen, fluorine, —OH, —NH₂, trifluoromethyl and methoxy, and both R⁵ and R^{5′} are hydrogen; or, R⁴ and R^{4′}, together with the carbon atom connecting R⁴ and R^{4′}, form a carbon-carbon double bond or a 3-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3-membered carbocyclic ring, and both R^{5′} and R^{4′} are hydrogen;
Ring A is selected from the group consisting of cyclopropyl, phenyl, furanyl, thienyl, pyrimidinyl and pyridinyl;
R³ is selected from the group consisting of hydrogen, fluorine, —OH, methoxy, —NH₂ and hydroxymethyl;
m is 1 or 2;
n is 5.

In some embodiments, in any of the above general formulas,
R¹ and R² are both methoxy;
R⁴ is hydrogen or fluorine, R^{4′} is selected from the group consisting of hydrogen, fluorine, —OH, —NH₂, trifluoromethyl and methoxy, and both R⁵ and R^{5′} are hydrogen; or, R⁴ and R^{4′}, together with the carbon atom connecting R⁴ and R^{4′}, form a carbon-carbon double bond or a 3-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3-membered carbocyclic ring, and both R^{5′} and R^{4′} are hydrogen;

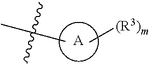

is selected from the group consisting of

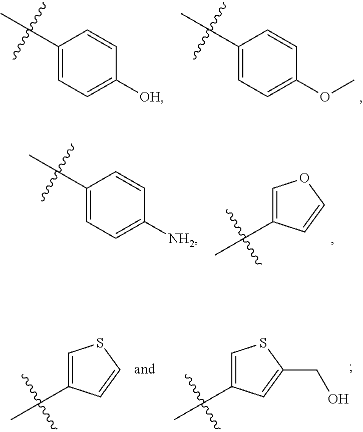

n is 5.

In some embodiments, the compound is selected from the group consisting of the following structures:

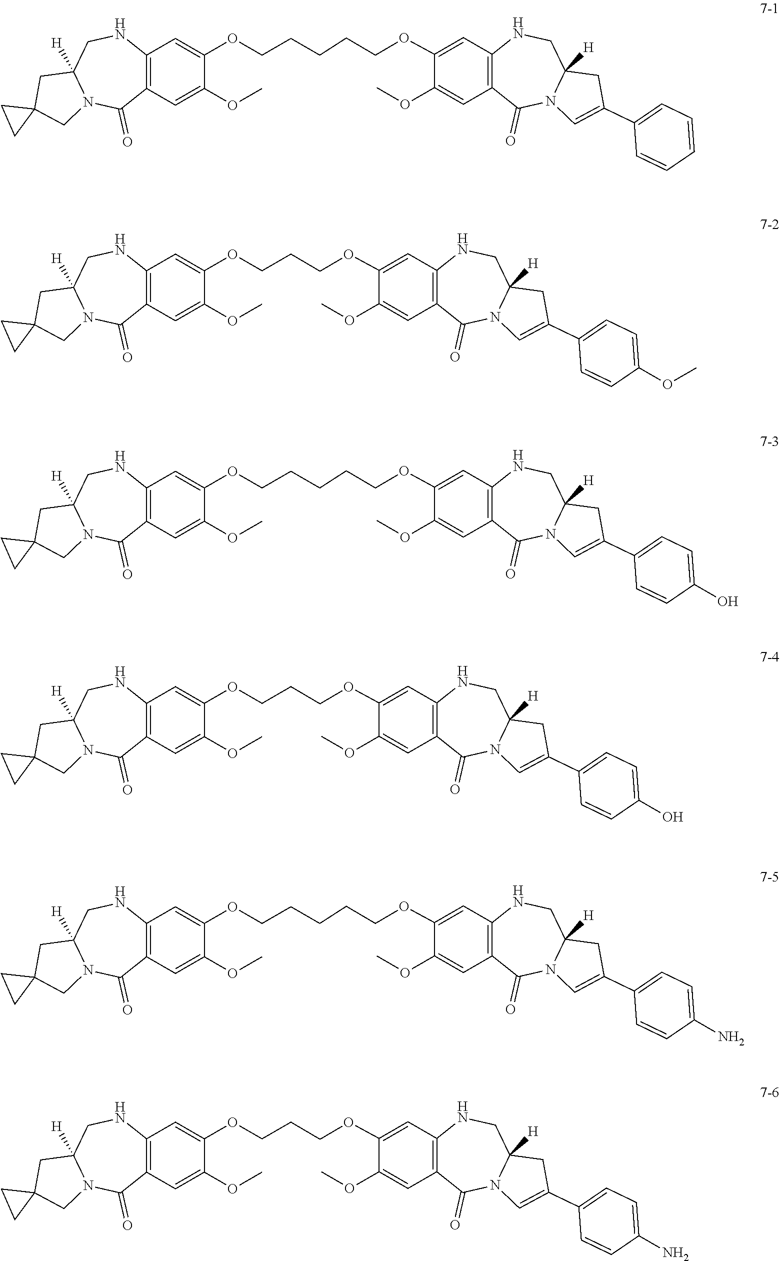

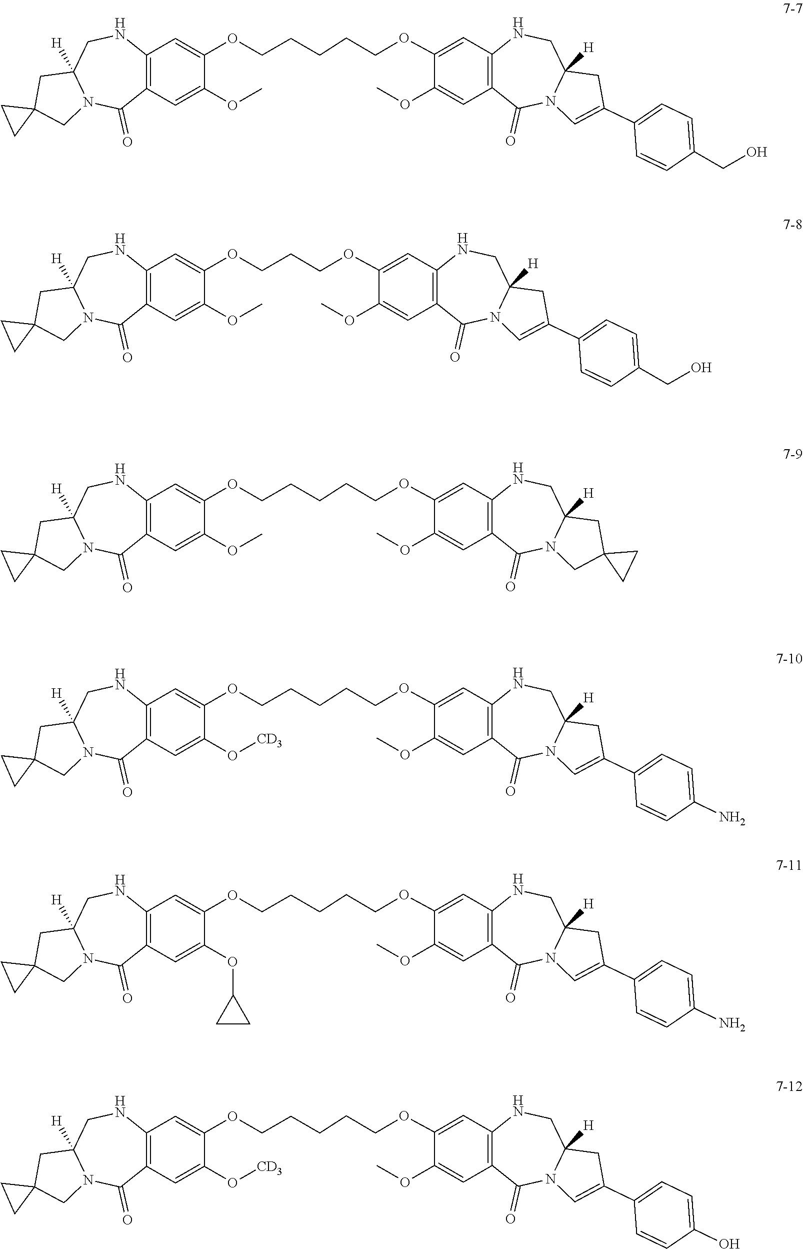

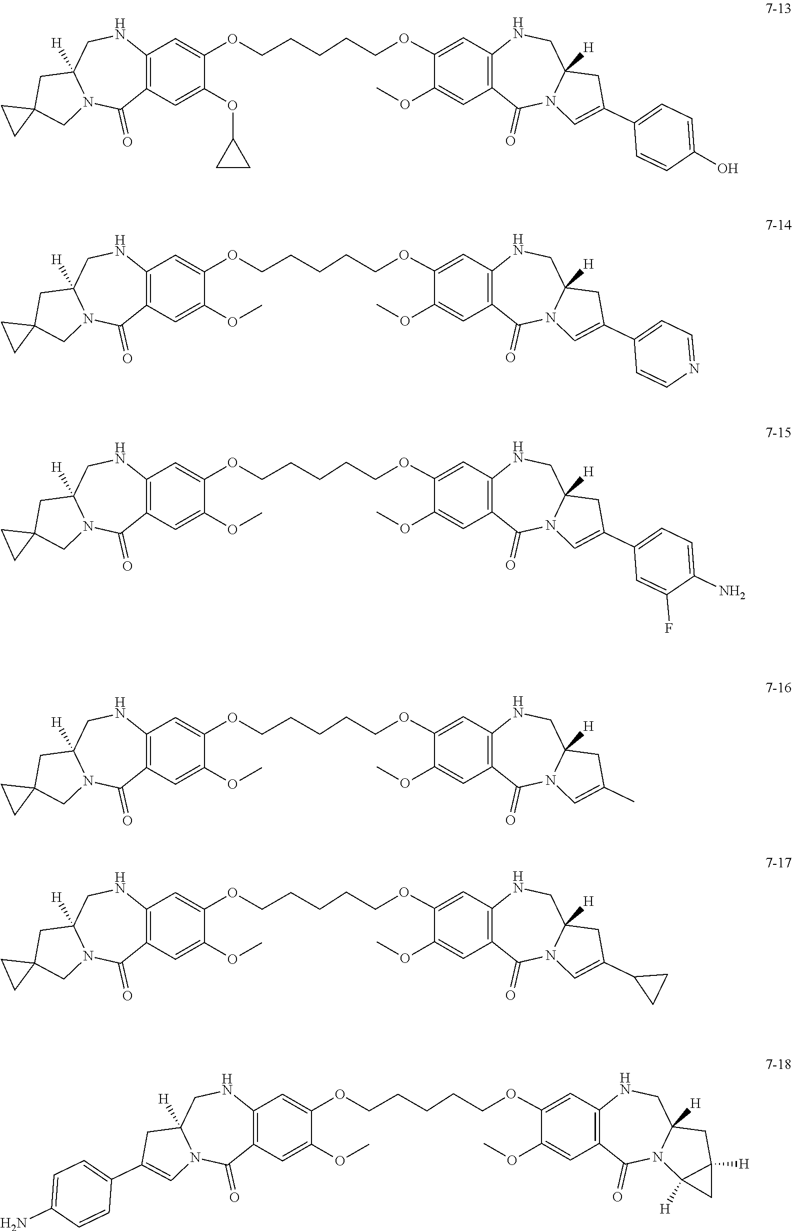

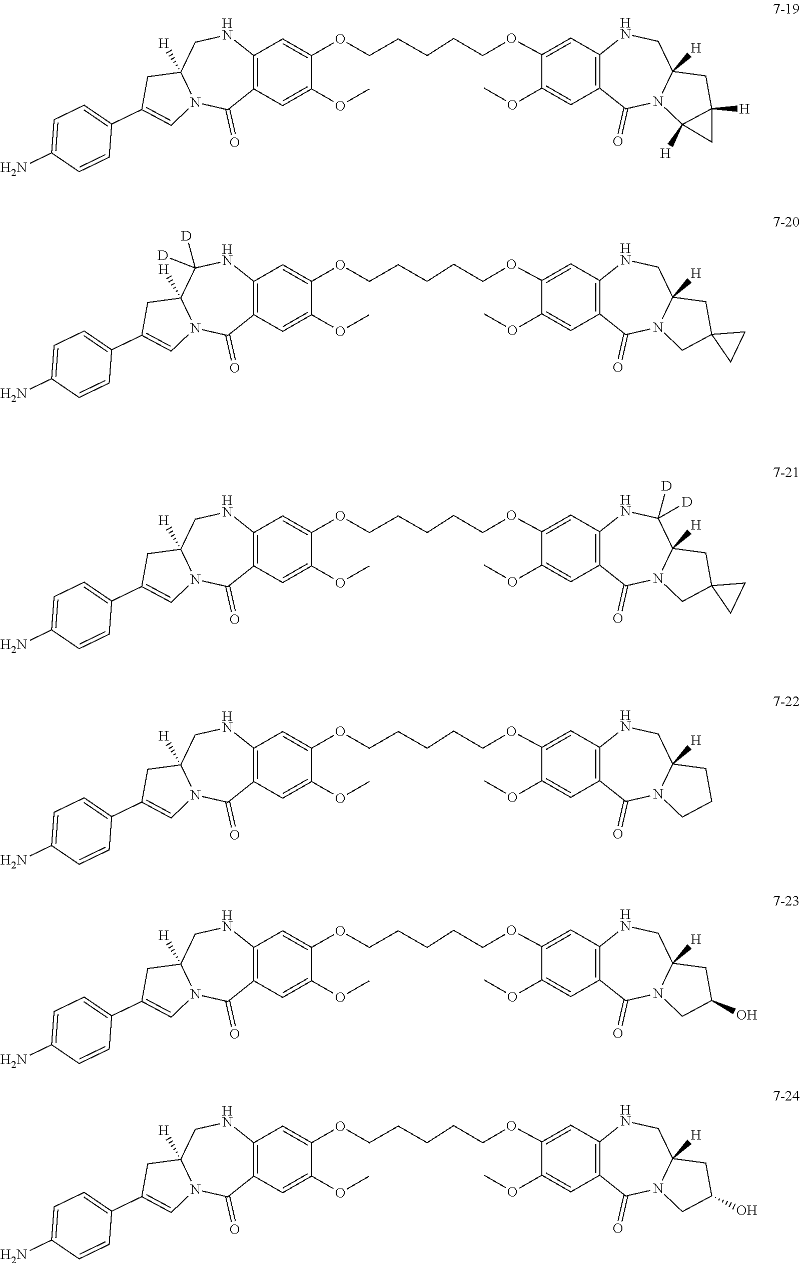

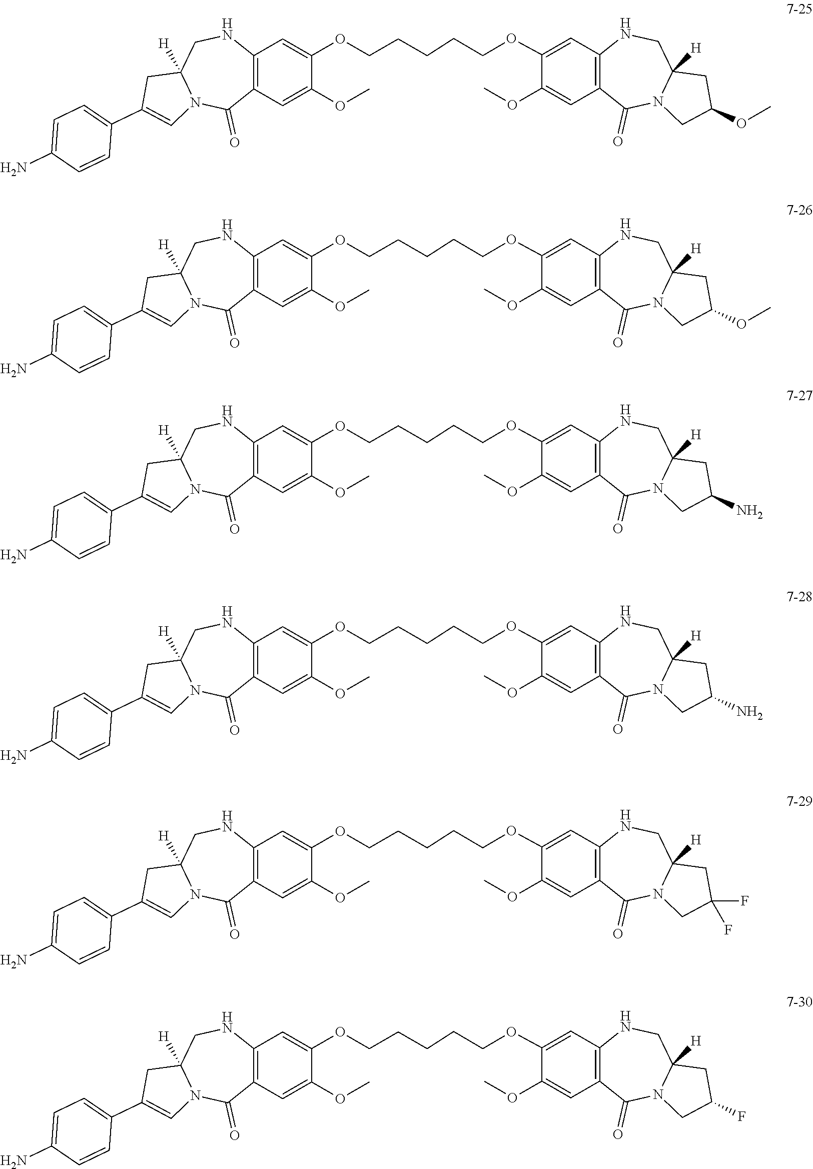

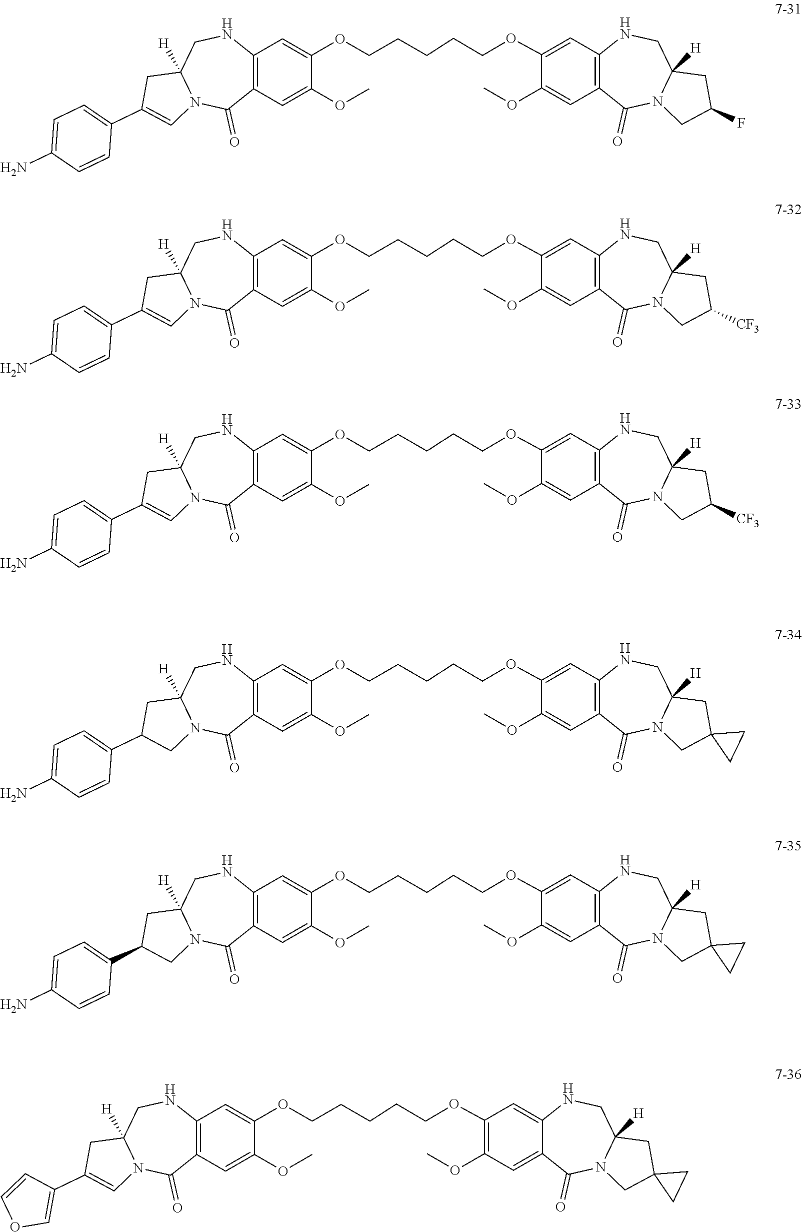

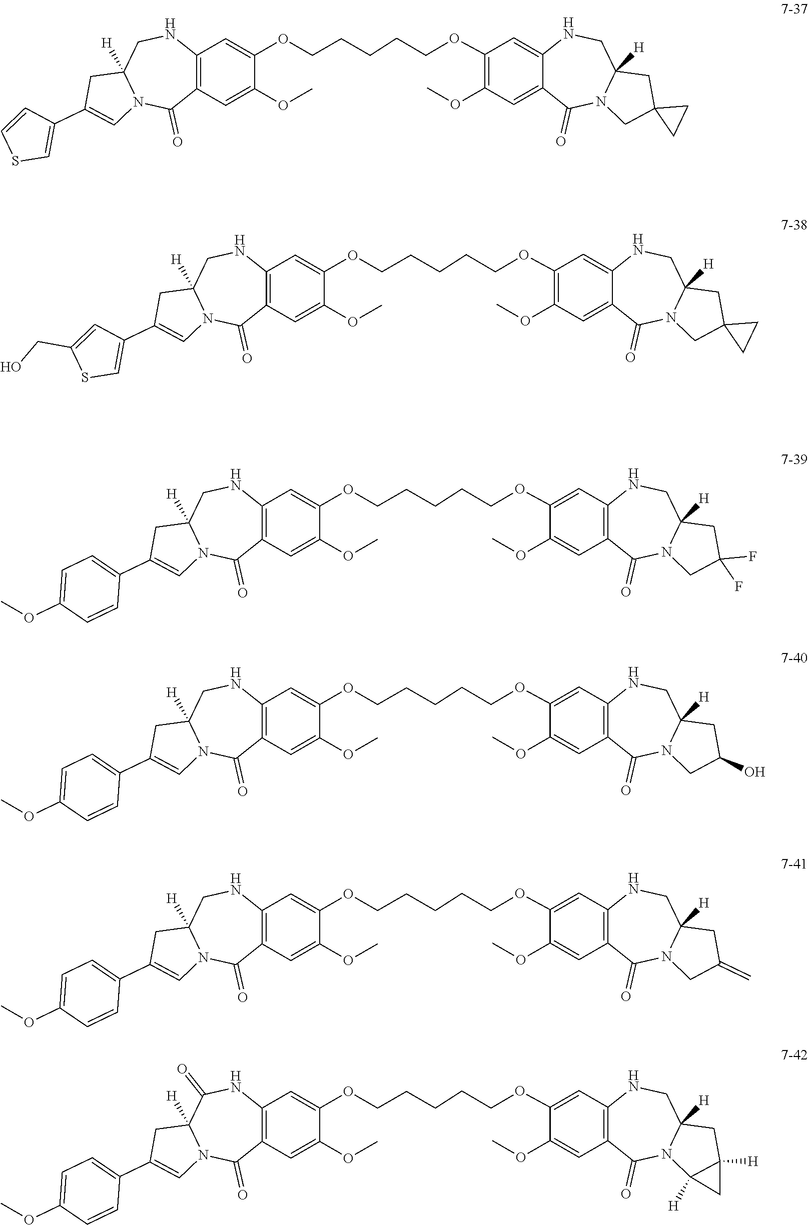

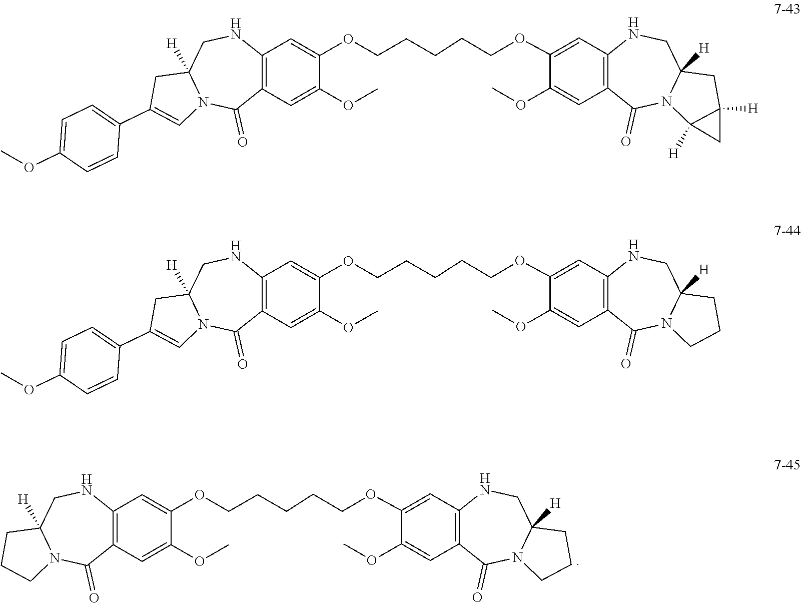 [SMILES: COc1ccc(C2=CN3C(=O)c4cc(OC)c(OCCCCCOc5cc6c(cc5OC)C(=O)N5[C@H](CN6)C[C@H]6C[C@H]65)cc4NC[C@@H]3C2)cc1 ; COc1ccc(C2=CN3C(=O)c4cc(OC)c(OCCCCCOc5cc6c(cc5OC)C(=O)N5CCC[C@H]5CN6)cc4NC[C@@H]3C2)cc1 ; COc1cc2c(cc1OCCCCCOc1cc3c(cc1OC)C(=O)N1CCC[C@H]1CN3)NC[C@@H]1CCCN1C2=O]

### Antibody-Drug Conjugates

The fused-ring compounds of the present invention have excellent anti-tumor activity and are suitable for preparing antibody-drug conjugates. In some embodiments, the obtained antibody-drug conjugates have good stability, homogeneity, hydrophilicity, efficacy and safety.

Therefore, in another aspect, the present invention provides an antibody-drug conjugate, which comprises a moiety of any of the compound or pharmaceutically acceptable salt, ester, stereoisomer, polymorph, solvate, N-oxide, isotopically-labeled substance, metabolite or prodrug thereof as described above.

### Pharmaceutical Composition

The second aspect of the present invention provides a pharmaceutical composition, which comprises the compound or pharmaceutically acceptable salt, ester, stereoisomer, polymorph, solvate, N-oxide, isotopically-labeled substance, metabolite or prodrug thereof as described above, or the antibody-drug conjugate as described above, and one or more pharmaceutical excipients.

The compound or antibody-drug conjugate as described herein is usually formulated together with a pharmaceutically acceptable parenteral vehicle into a unit injectable form for parenteral use, such as bolus injection, intravenous injection, intratumoral injection, etc. Optionally, the antibody-drug conjugate having the desired purity is mixed with a pharmaceutically acceptable diluent, carrier, excipient or stabilizer in the form of a lyophilized agent or solution (Remington's Pharmaceutical Sciences (1980) 16^{th} edition, Osol, A. Ed.). The compound or antibody-drug conjugate or pharmaceutical composition containing the same as described herein can be administered by any route suitable for the individual to be treated.

### Use

The third aspect of the present invention provides a use of the compound or pharmaceutically acceptable salt, ester, stereoisomer, polymorph, solvate, N-oxide, isotopically-labeled substance, metabolite or prodrug thereof, the antibody-drug conjugate, or the pharmaceutical composition as described above in the manufacture of a medicament for treating a cancer or tumor.

The fourth aspect of the present invention provides the compound or pharmaceutically acceptable salt, ester, stereoisomer, polymorph, solvate, N-oxide, isotopically-labeled substance, metabolite or prodrug thereof, the antibody-drug conjugate, or the pharmaceutical composition as described above, for use in treating a cancer or tumor.

The fifth aspect of the present invention provides a method for treating cancer or tumor, comprising administering to an individual in need thereof a therapeutically effective amount of the compound or pharmaceutically acceptable salt, ester, stereoisomer, polymorph, solvate, N-oxide, isotopically-labeled substance, metabolite or prodrug thereof, the antibody-drug conjugate, or the pharmaceutical composition as described above.

In some embodiments of the present invention, the cancer or tumor is a solid tumor or a hematological malignancy, such as gastric cancer, breast cancer, lung cancer (e.g., non-small cell lung cancer, specifically lung adenocarcinoma) or urothelial carcinoma.

In some embodiments, the compound or pharmaceutical composition thereof is sufficient (e.g., in a subject) to:
(1) inhibit the proliferation of cells (e.g., tumor cells);
(2) inhibit tumor growth; or
(3) induce and/or increase antibody-dependent cell-mediated cytotoxicity activity;
(4) any combination of the above (1) to (3).

### Definitions

Unless otherwise defined below, the meanings of all technical terms and scientific terms used herein are intended to be the same as those generally understood by those skilled in the art. References to the techniques used herein are intended to refer to techniques generally understood in the art, including those changes in techniques or replacements with equivalent techniques that are obvious to those skilled in the art. In addition, the laboratory operation steps of genomics, nucleic acid chemistry, molecular biology, etc. used herein are all routine steps widely used in the corresponding fields. Although it is believed that the following terms are well understood by those skilled in the art, the following definitions are still set forth to better explain the present invention.

The term “antibody” refers to an immunoglobulin molecule that is typically composed of two pairs of polypeptide chains, each pair having one light chain (LC) and one heavy chain (HC). Antibody light chains can be classified as κ (kappa) and λ (lambda) light chains. Heavy chains can be classified as μ, δ, γ, α, or ε, and define the antibody's isotype as IgM, IgD, IgG, IgA, and IgE, respectively. Within the light and heavy chains, the variable and constant regions are connected by a “J” region of about 12 or more amino acids, and the heavy chain also contains a “D” region of about 3 or more amino acids. Each heavy chain consists of a heavy chain variable region (VH) and a heavy chain constant region (CH). The heavy chain constant region consists of three domains (CH1, CH₂, and CH₃). Each light chain consists of a light chain variable region (VL) and a light chain constant region (CL). The light chain constant region consists of one domain, CL. The constant domain is not directly involved in the binding of antibodies to antigens, but exhibits a variety of effector functions, such as mediating the binding of immunoglobulins to host tissues or factors, including various cells of the immune system (e.g., effector cells) and the first component (C1q) of the classical complement system. The VH and VL regions can also be subdivided into regions of high variability (called complementarity determining regions (CDRs)), interspersed with more conservative regions called framework regions (FRs). Each VH and VL consists of three CDRs and four FRs arranged from the amino terminus to the carboxyl terminus in the following order: FR1, CDR1, FR2, CDR2, FR3, CDR3, FR4. The variable regions (VH and VL) of each heavy chain/light chain pair form antigen binding sites, respectively. The allocation of amino acids in each region or domain can follow various numbering systems known in the art.

The term “complementarity determining region” or “CDR” refers to the amino acid residues in the variable region of an antibody that are responsible for antigen binding. The variable regions of the heavy and light chains each contain three CDRs, designated CDR1, CDR2 and CDR3. The precise boundaries of these CDRs can be defined according to various numbering systems known in the art, such as the Kabat numbering system (Kabat et al., Sequences of Proteins of Immunological Interest, 5th Ed. Public Health Service, National Institutes of Health, Bethesda, Md., 1991), the Chothia numbering system (Chothia & Lesk (1987) J. Mol. Biol. 196:901-917; Chothia et al. (1989) Nature 342:878-883), the IMGT numbering system (Lefranc et al., Dev. Comparat. Immunol. 27:55-77, 2003), or the AbM numbering system (Martin A C R, Cheetham J C, Rees A R (1989) Modelling antibody hypervariable loops: A combined algorithm. Proc Natl Acad Sci USA 86:9268-9272). For a given antibody, a person skilled in the art will easily identify the CDRs defined by each numbering system. Moreover, the correspondence between different numbering systems is well known to those skilled in the art (for example, see Lefranc et al., Dev. Comparat. Immunol. 27:55-77, 2003).

In the present invention, the CDRs contained in an antibody or antigen-binding fragment thereof can be determined according to various numbering systems known in the art, such as by the Kabat, Chothia, IMGT or AbM numbering system. In certain embodiments, the CDRs contained in an antibody or antigen-binding fragment thereof are defined by the Chothia numbering system.

The term “framework region” or “FR” residues refers to those amino acid residues in the variable region of an antibody other than the CDR residues defined above.

The term “antigen-binding fragment” of antibody refers to a polypeptide of a fragment of an antibody, such as a polypeptide of a fragment of a full-length antibody, which retains the ability to specifically bind to the same antigen bound by the full-length antibody, and/or competes with the full-length antibody for specific binding to the antigen, which is also referred to as an “antigen-binding portion”. See generally, Fundamental Immunology, Ch. 7 (Paul, W., ed., 2nd ed., Raven Press, N.Y. (1989), which is incorporated herein by reference in its entirety for all purposes. Antigen-binding fragments of antibodies can be produced by recombinant DNA techniques or by enzymatic or chemical cleavage of intact antibodies. Non-limiting examples of antigen-binding fragments include Fab fragments, Fab′ fragments, F(ab)′₂ fragments, F(ab)′₃ fragments, Fd, Fv, scFv, di-scFv, (scFv)₂, disulfide-stabilized Fv proteins (“dsFv”), single domain antibodies (sdAb, nanobodies), and polypeptides that contain at least a portion of an antibody sufficient to confer specific antigen binding ability to the polypeptide. Engineered antibody variants are reviewed in Holliger et al., 2005; Nat Biotechnol, 23: 1126-1136.

The term “Fd” refers to an antibody fragment consisting of VH and CH1 domains; the term “dAb fragment” refers to an antibody fragment consisting of VH domain (Ward et al., Nature 341:544 546 (1989)). The term “Fab fragment” refers to an antibody fragment consisting of VL, VH, CL and CH1 domains. The term “F(ab′)₂ fragment” refers to an antibody fragment comprising two Fab fragments connected by a disulfide bridge on the hinge region. The term “Fab′ fragment” refers to a fragment obtained after reducing the disulfide bonds connecting the two heavy chain fragments in the F(ab′)₂ fragment, which consists of a complete light chain and the Fd fragment of the heavy chain (consisting of VH and CH1 domains).

The term “Fv” refers to an antibody fragment consisting of the VL and VH domains of a single arm of an antibody. The Fv fragment is generally considered to be the smallest antibody fragment that can form a complete antigen binding site. It is generally believed, six CDRs confer antigen-binding specificity to an antibody. However, even a single variable region (e.g., an Fd fragment, which contains only three CDRs specific for an antigen) is able to recognize and bind to the antigen, although its affinity may be lower than that of the complete binding site.

The term “Fc” refers to an antibody fragment formed by binding the second and third constant regions of the first heavy chain of the antibody and the second and third constant regions of the second heavy chain of the antibody via disulfide bonds. The Fc fragment of an antibody has a variety of different functions but is not involved in antigen binding.

The term “scFv” refers to a single polypeptide chain comprising VL and VH domains, wherein the VL and VH are connected via a linker (see, for example, Bird et al., Science 242:423-426 (1988); Huston et al., Proc. Natl. Acad. Sci. USA 85:5879-5883 (1988); and Pluckthun, The Pharmacology of Monoclonal Antibodies, Vol. 113, Roseburg and Moore, eds., Springer-Verlag, New York, pp. 269-315 (1994)). Such scFv molecules may have the general structure: NH₂—VL-linker-VH—COOH or NH₂—VH-linker-VL-COOH. Suitable prior art linkers consist of repeated GGGGS amino acid sequences or variants thereof. For example, a linker having the amino acid sequence (GGGGS)₄ may be used, but variants thereof may also be used (Holliger et al. (1993), Proc. Natl. Acad. Sci. USA 90: 6444-6448). Other linkers that can be used in the present invention are described by Alfthan et al. (1995), Protein Eng. 8:725-731, Choi et al. (2001), Eur. J. Immunol. 31: 94-106, Hu et al. (1996), Cancer Res. 56:3055-3061, Kipriyanov et al. (1999), J. Mol. Biol. 293:41-56 and Roovers et al. (2001), Cancer Immunol. In some cases, a disulfide bond may also exist between the VH and VL of the scFv. In certain embodiments, the VH and VL domains may be positioned relative to each other in any suitable arrangement. For example, a scFv may comprises NH₂—VH—VH—COOH, or NH₂—VL-VL-COOH.

The term “single-domain antibody” (sdAb) has the meaning generally understood by those skilled in the art, which refers to an antibody fragment consisting of a single monomeric variable antibody domain (e.g., a single heavy chain variable region) that retains the ability to specifically bind to the same antigen as the full-length antibody (Holt, L. et al., Trends in Biotechnology, 21(11):484-490, 2003). Single-domain antibody is also called nanobody.

Each of the above antibody fragments retains the ability to specifically bind to the same antigen bound by the full-length antibody, and/or competes with the full-length antibody for specific binding to the antigen.

As used herein, unless the context clearly indicates otherwise, when referring to the term “antibody”, it includes not only complete antibodies but also antigen-binding fragments of antibodies.

An antigen-binding fragments of an antibody (e.g., the aforementioned antibody fragments) can be obtained from a given antibody (e.g., the antibody provided by the present invention) using conventional techniques known to those skilled in the art (e.g., recombinant DNA technology or enzymatic or chemical cleavage methods), and the antigen-binding fragments of antibodies can be screened for specificity in the same manner as for complete antibodies.

The writing of the twenty conventional amino acids involved herein follows conventional usage. See, for example, Immunology-A Synthesis (2nd Edition, E. S. Golub and D. R. Gren, Eds., Sinauer Associates, Sunderland, Mass. (1991)), which is incorporated herein by reference. In the present invention, amino acids are generally represented by single-letter and three-letter abbreviations known in the art. For example, alanine can be represented by A or Ala.

The term “linker” refers to a structural fragment that connects a cytotoxic drug to an antibody or antigen-binding fragment thereof.

The term “drug-linker complex” refers to a structure formed by connecting the cytotoxic drug and the linker (as described in the present invention) prior to its conjugation to an antibody or antigen-binding fragment thereof. The “drug-linker complex” is covalently linked to an antibody or antigen-binding fragment thereof to obtain the antibody-drug conjugate described in the present application.

The “drug-linker complex” also includes all pharmaceutically acceptable isotopically-labeled substances thereof, which are identical to the “drug-linker complex” compound of the present invention, except that one or more atoms are replaced by isotopes (atoms having the same atomic number but an atomic mass or mass number different from the atomic mass or mass number that predominates in nature). Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ³⁷Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O and ¹⁸O); and isotopes of sulfur (e.g., ³⁵S).

The terms “include”, “comprise”, “have”, “contain” or “involve” and other variant forms thereof herein are inclusive or open-ended and do not exclude other unlisted elements or method steps.

The term “alkyl” refers to a group obtained by removing one hydrogen atom from a straight or branched hydrocarbon group, such as “C₁₋₂₀ alkyl”, “C₁₋₁₀ alkyl”, “C₁₋₆ alkyl”, “C₁₋₄ alkyl”, “C₁₋₃ alkyl”, etc. Specific examples include, but are not limited to: methyl, ethyl, n-propyl, isopropyl, n-butyl, isobutyl, sec-butyl, tert-butyl, n-pentyl, isopentyl, 2-methylbutyl, neopentyl, 1-ethylpropyl, n-hexyl, isohexyl, 3-methylpentyl, 2-methylpentyl, 1-methylpentyl, 3,3-dimethylbutyl, 2,2-dimethylbutyl, 1,1-dimethylbutyl, 1,2-dimethylbutyl, 1,3-dimethylbutyl, 2,3-dimethylbutyl, 2-ethylbutyl, 1,2-dimethylpropyl, etc.

As used herein, the term “haloalkyl” refers to an alkyl substituted with one or more (e.g., 1 to 3) identical or different halogen atoms. The term “C₁₋₄ haloalkyl” refers to a C₁₋₄ alkyl substituted with one or more identical or different halogen atoms, such as —CF₃, —C₂F₅, —CHF₂, —CH₂F, —CH₂CF₃, —CH₂Cl or —CH₂CH₂CF₃, etc.

As used herein, the term “hydroxyalkyl” refers to a group formed by replacing hydrogen atoms in an alkyl with one or more hydroxyl groups, such as C₁₋₄ hydroxyalkyl or C₁₋₆ hydroxyalkyl, examples of which include but are not limited to hydroxymethyl, hydroxyethyl, hydroxypropyl, hydroxybutyl, —CH(OH)CH₃, etc.

As used herein, the term “alkenyl” refers to a straight or branched aliphatic hydrocarbonyl having one or more carbon-carbon double bonds. For example, the term “C₂₋₆ alkenyl” as used herein refers to an alkenyl having 2 to 6 carbon atoms and one, two or three carbon-carbon double bonds (e.g., vinyl, 1-propenyl, 2-propenyl, 2-butenyl, 3-butenyl, 2-pentenyl, 3-pentenyl, 4-pentenyl, 2-hexenyl, 3-hexenyl, 4-hexenyl, 5-hexenyl, 2-methyl-2-propenyl, 4-methyl-3-pentenyl, etc.), which is optionally substituted with one or more (e.g., 1 to 3) substituents described herein.

As used herein, the term “alkynyl” refers to a straight or branched aliphatic hydrocarbonyl having one or more carbon-carbon triple bonds. For example, the term “C₂₋₆ alkynyl” as used herein refers to an alkynyl having 2 to 6 carbon atoms and one, two or three carbon-carbon triple bonds (e.g., ethynyl, 1-propynyl, 2-propynyl, 2-butynyl, 3-butynyl, 2-pentynyl, 3-pentynyl, 4-pentynyl, 2-hexynyl, 3-hexynyl, 4-hexynyl, 5-hexynyl, etc.), which is optionally substituted with one or more (e.g., 1 to 3) substituents described herein.

As used herein, the term “alkoxy” refers to a group in which an alkyl (as defined above) is attached to the rest of molecule via an oxygen atom, such as C₁₋₈ alkoxy, C₁₋₆ alkoxy, C₁₋₄ alkoxy or C₁₋₃ alkoxy. Representative examples of C₁₋₆ alkoxy include, but are not limited to, methoxy, ethoxy, propoxy, isopropoxy, n-propoxy, isopropoxy, n-butoxy, isobutoxy, tert-butoxy, pentyloxy, hexyloxy, and the like, wherein the alkoxy is optionally substituted with one or more (e.g., 1 to 3) identical or different substituents. For example, the term “haloalkoxy” means that the hydrogen atoms of the alkoxy are substituted with one or more (e.g., 1 to 3) identical or different halogen atoms.

As used herein, the term “cycloalkyl” refers to a saturated or unsaturated non-aromatic monocyclic or polycyclic (e.g., bicyclic) hydrocarbon ring group, including but not limited to monocyclic alkyl (e.g., cyclopropyl, cyclobutyl, cyclopentyl, cyclohexyl, cycloheptyl, cyclooctyl, cyclononyl, etc.) and bicyclic alkyl, including spiro, fused (condensed) or bridged ring systems (i.e., spiro cycloalkyl, fused (condensed) cycloalkyl, and bridged cycloalkyl, such as bicyclo[1.1.1]pentyl, bicyclo[2.2.1]heptyl, etc.). In the present invention, the cycloalkyl is optionally substituted with one or more (e.g., 1 to 3) identical or different substituents. The carbon atoms on the cycloalkyl are optionally substituted with oxo (i.e., forming C═O). The term “C₃₋₈ cycloalkyl” refers to a cycloalkyl having 3 to 8 ring carbon atoms, such as C₃₋₆ cycloalkyl, which can be a monocyclic alkyl, such as cyclopropyl, cyclobutyl, cyclopentyl, cyclohexyl, cycloheptyl or cyclooctyl, or a bicyclic alkyl, such as C₅₋₈ spiro cycloalkyl, C₅₋₈ bridged cycloalkyl, C₅₋₈ fused cycloalkyl, C₅₋₆ spiro cycloalkyl, C₅₋₆ bridged cycloalkyl or C₅₋₆ fused cycloalkyl. The term “aliphatic ring” refers to a saturated or unsaturated carbon ring containing 3 or more carbon atoms connected together, and typical aliphatic rings include, for example, cyclopropane ring, cyclobutane ring, cyclopentane ring, cyclohexane ring, etc.

As used herein, the term “heterocyclyl” or “heterocyclic ring” refers to an aliphatic saturated or partially unsaturated monocyclic or polycyclic (e.g., fused, spiro or bridged cyclic) group having 2 or more (e.g., 3, 4, 5, 6, 7, 8, 9, 10, 11, 12, 13 or 14) carbon atoms, and one or more (e.g., 1, 2, 3 or 4) heteroatoms, including but not limited to oxygen atoms, nitrogen atoms and sulfur atoms, the carbon atoms and heteroatoms on the heterocyclyl are optionally substituted with oxo groups (e.g., forming C═O, S(═O) or S(═O)₂), or optionally substituted with one or more (e.g., 1 to 3) substituents independently selected from the group consisting of halogen and C₁₋₃ alkyl.

As used herein, the term “aryl” or “aromatic ring” refers to an all-carbon monocyclic or fused polycyclic aromatic group having a conjugated n electron system. As used herein, the term “C₆₋₁₀ aryl” or “aromatic ring” refers to an aryl or aromatic ring containing 6 to 10 carbon atoms, such as phenyl or benzene ring, naphthyl or naphthalene ring. The aryl is optionally substituted with one or more (e.g., 1 to 3) identical or different substituents (e.g., halogen, OH, CN, NO₂, C₁₋₆ alkyl, etc.).

As used herein, the term “heteroaryl” or “heteroaromatic ring” refers to a monocyclic or polycyclic aromatic group containing one or more identical or different heteroatoms, including monocyclic heteroaryls and bicyclic or polycyclic ring systems containing at least one heteroaromatic ring (aromatic ring system containing at least one heteroatom), which may have 5, 6, 7, 8, 9, 10, 11, 12, 13 or 14 ring atoms, such as 5, 6, 7, 8, 9 or 10 ring atoms. The heteroatom may be oxygen, nitrogen or sulfur. The carbon atoms and heteroatoms on the heteroaryl are optionally substituted with oxo groups (e.g., forming C═O, S(═O) or S(═O)₂).

As used herein, the term “halo” or “halogen” group is defined to include F, Cl, Br or I.

The term “isotopically-labeled substance” means that the compound is identical to the compound of the present invention except that one or more atoms are replaced by isotopes (atoms having the same atomic number but an atomic mass or mass number different from the atomic mass or mass number that predominates in nature). Examples of isotopes suitable for inclusion in the present invention include, but are not limited to, isotopes of hydrogen (e.g., ²H, ³H, deuterium D, tritium T); isotopes of carbon (e.g., ¹¹C, ¹³C, and ¹⁴C); isotopes of chlorine (e.g., ₃₇Cl); isotopes of fluorine (e.g., ¹⁸F); isotopes of iodine (e.g., ¹²³I and ¹²⁵I); isotopes of nitrogen (e.g., ¹³N and ¹⁵N); isotopes of oxygen (e.g., ¹⁵O, ¹⁷O, and ¹⁸O); and isotopes of sulfur (e.g., ³⁵S).

The term “substitute” refers to the replacement of one or more (e.g., 1, 2, 3, 4, or 5) hydrogens on the specified compound or structural fragment by a substituent, provided that the normal atomic valence of the specified atom in the current context is not exceeded and the substitution forms a stable compound. Combinations of substituents and/or variables are permitted only if such combinations form stable compounds. For example, the substituents are each independently composed of one or more of the following structures: —O—, —S—, —NR′—, halogen, —CN, —OH, —NH₂, —NO₂, —CN, ═O, C₁-C₆ alkyl or alkylene, C₁-C₆ halo alkyl or alkylene, C₁-C₆ alkoxy, C₂-C₆ alkenyl or alkenylene, C₂-C₆ alkynyl or alkynylene, C₃-C₈ cycloalkyl or cycloalkylene, 3- to 8-membered heterocyclyl or heterocyclylene, C₆-C₁₀ aryl or arylene, and 5- to 10-membered heteroaryl or heteroarylene, etc.

If a substituent is described as “independently selected from” a group, each substituent is selected independently of the other. Therefore, each substituent may be the same as or different from another (other) substituent.

As used herein, the term “one or more” means 1 or more than 1 under reasonable conditions, such as 2, 3, 4, 5, 6, 7, 8, 9 or 10.

Unless otherwise specified, as used herein, the point of attachment of a substituent may be from any suitable position of the substituent.

The term “stereoisomer” refers to an isomer formed due to at least one asymmetric center. For a compound having one or more (e.g., one, two, three, or four) asymmetric centers, it may produce a racemic mixture, a single enantiomer, a diastereomeric mixture, and individual diastereomers. Specific individual molecules may also exist as geometric isomers (cis/trans). Similarly, the compounds of the present invention may exist as mixtures of two or more structurally different forms in rapid equilibrium (commonly referred to as tautomers). Representative examples of tautomers include keto-enol tautomers, phenol-keto tautomers, nitroso-oxime tautomers, imine-enamine tautomers, and the like. For example, nitroso-oxime may exist in equilibrium in a solution in the following tautomeric forms:

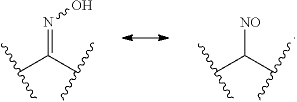

It is to be understood that the scope of the present application encompasses all such isomers in any proportion (e.g., 60%, 65%, 70%, 75%, 80%, 85%, 90%, 95%, 96%, 97%, 98%, 99%) or mixtures thereof.

Solid line (—), solid wedge ( [symbol] ), or dashed wedge ( [symbol] ) may be used herein to depict a chemical bond of the compound of the present invention. The use of a solid line to depict a bond to an asymmetric carbon atom is intended to indicate that all possible stereoisomers (e.g., specific enantiomers, racemic mixtures, etc.) at that carbon atom are included. The use of solid or dashed wedges to depict bonds to asymmetric carbon atoms is intended to indicate that the indicated stereoisomer is present. When present in a racemic mixture, solid and dashed wedges are used to define relative stereochemistry, rather than absolute stereochemistry. Unless otherwise specified, the compounds of the present invention are intended to exist in the form of stereoisomers (including cis and trans isomers, optical isomers (e.g., R and S enantiomers), diastereomers, geometric isomers, rotational isomers, conformational isomers, atropisomers and mixtures thereof). The compounds of the present invention may exhibit more than one type of isomerism and consist of mixtures thereof (e.g., racemic mixtures and diastereoisomer pairs).

The present invention encompasses all possible crystalline forms or polymorphs of the compounds of the present invention, which may be a single polymorph or a mixture of more than one polymorph in any proportion.

Cocrystal refers to a crystal composed of a pharmaceutically active molecule and other physiologically acceptable acid, base, salt, or non-ionic compound molecules, bound within the same crystal lattice through hydrogen bonds, x-n stacking, van der Waals forces, or other non-covalent interactions.

It should also be understood that certain compounds of the present invention may exist in their free form for treatment, or, where appropriate, in the form of pharmaceutically acceptable derivatives thereof. In the present invention, pharmaceutically acceptable derivatives include, but are not limited to, pharmaceutically acceptable salts, esters, solvates, N-oxides, metabolites or prodrugs, which can directly or indirectly provide the compounds of the present invention or their metabolites or residues after being administered to patients in need thereof. Therefore, when referring to “compound of the present invention” herein, it is also intended to cover the above-mentioned various derivative forms of the compound.

Pharmaceutically acceptable salts of the compound of the present invention include acid addition salts and base addition salts thereof, for example, hexafluorophosphate, meglumine salt, etc. For a review of suitable salts, see Stahl and Wermuth's “Handbook of Pharmaceutical Salts: Properties, Selection, and Use” (Wiley-VCH, 2002).

As used herein, the term “ester” refers to an ester derived from the compound of the general formula in the present application, including a physiologically hydrolyzable ester (which can be hydrolyzed under physiological conditions to release the compound of the present invention in free acid or alcohol form). The compound of the present invention itself may also be an ester.

The compound of the present invention may exist in the form of solvate, preferably hydrate, wherein the compound of the present invention contains a polar solvent, in particular water, methanol or ethanol, as a structural element of the crystal lattice of the compound. The amount of polar solvent, in particular water, may be present in a stoichiometric or non-stoichiometric ratio.

Those skilled in the art will appreciate that not all nitrogen-containing heterocycles are capable of forming N-oxides, as the nitrogen atom must have an available lone pair of electrons for oxidation to occur. Those skilled in the art will recognize nitrogen-containing heterocycles capable of forming N-oxides. Those skilled in the art will also recognize that tertiary amines are capable of forming N-oxides. Synthetic methods for preparing N-oxides of heterocycles and tertiary amines are well known to those skilled in the art, including but not limited to oxidation of heterocycles and tertiary amines with peroxyacids such as peracetic acid and meta-chloroperbenzoic acid (MCPBA), hydrogen peroxide, alkyl hydroperoxides such as tert-butyl hydroperoxide, sodium perborate, and dioxiranes such as dimethyldioxirane. These methods for preparing N-oxides have been extensively described and reviewed in the literature, see, for example, T. L. Gilchrist, Comprehensive Organic Synthesis, vol. 7, pp 748-750; A. R. Katritzky and A. J. Boulton, Eds., Academic Press; and G. W. H. Cheeseman and E. S. G. Werstiuk, Advances in Heterocyclic Chemistry, vol. 22, pp 390-392, A. R Katritzky and A. J. Boulton, Eds., Academic Press.

Also included within the scope of the present invention are metabolites of the compound of the present invention, i.e., substances formed in vivo upon administration of the compound of the present invention. Such products may be produced, for example, by oxidation, reduction, hydrolysis, amidation, deamidation, esterification, enzymatic hydrolysis, etc. of the administered compound. Thus, the present invention includes metabolites of the compound of the present invention, including a compound prepared by contacting the compound of the present invention with a mammal for a period of time sufficient to produce a metabolic product thereof.

The present invention further includes within its scope prodrugs of the compound of the invention, which are certain derivatives of the compound of the invention which themselves may have little or no pharmacological activity but which, when administered into or onto the body, are converted, for example, by hydrolytic cleavage, into the compound of the present invention having the desired activity. Typically, such prodrugs will be functional derivatives of the compound which can be readily converted in vivo into the desired therapeutically active compound. Additional information on the use of prodrugs can be found in “Pro-drugs as Novel Delivery Systems”, Vol. 14, ACS Symposium Series (T. Higuchi and V. Stella). The prodrugs of the present invention can be prepared, for example, by replacing appropriate functional groups present in the compound of the present invention with certain moieties known to those skilled in the art as “pro-moieties” (e.g., as described in “Design of Prodrugs”, H. Bundgaard (Elsevier, 1985)).

The present invention also covers compounds of the present invention containing protecting groups. In any process for preparing the compounds of the present invention, it may be necessary and/or desirable to protect sensitive or reactive groups on any of the molecules involved, thereby forming chemically protected forms of the compounds of the present invention. This can be achieved by conventional protecting groups, for example, as described in T. W. Greene & P. G. M. Wuts, Protective Groups in Organic Synthesis, John Wiley & Sons, 1991, which are incorporated herein by reference. The protecting groups may be removed at an appropriate subsequent stage using methods known in the art.

If a functional group or structural fragment is described as “substituted or unsubstituted” or “optionally substituted with . . . ”, the functional group or structural fragment may be (1) unsubstituted or (2) substituted.

### SPECIFIC MODELS FOR CARRYING OUT THE PRESENT INVENTION

The present invention is further described below by describing specific embodiments, but this is not a limitation of the present invention. Those skilled in the art can make various modifications or improvements based on the teachings of the present invention without departing from the basic idea and scope of the present invention.

The structures of the compounds described in the following examples were determined by nuclear magnetic resonance (¹H NMR) or mass spectrometry (MS).

Nuclear magnetic resonance (¹H NMR) was measured using a Bruker 400 MHz nuclear magnetic resonance instrument; the deuterated reagent was hexadeuterated dimethylsulfoxide (DMSO-d6); the internal standard substance was tetramethylsilane (TMS).

The abbreviations in the nuclear magnetic resonance (NMR) spectra used in the examples are shown below.

s: singlet, d: doublet, t: triplet, q: quartet, m: multiplet, br: broad, J: coupling constant, Hz: Hertz, DMSO-d6: deuterated dimethyl sulfoxide. δ values are expressed in ppm.

The mass spectrometer (MS) was determined using an Agilent (ESI) mass spectrometer, model: Agilent 6120B.

### Preparation Example 1: (S)-8-((5-(((S)-2-(4-Aminophenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11-tetrahydro-3H,5H-spiro[benzo[e]pyrrolo[1,2-a][1,4]diazepin-2,1′-cyclopropane]-5-one (7-5)

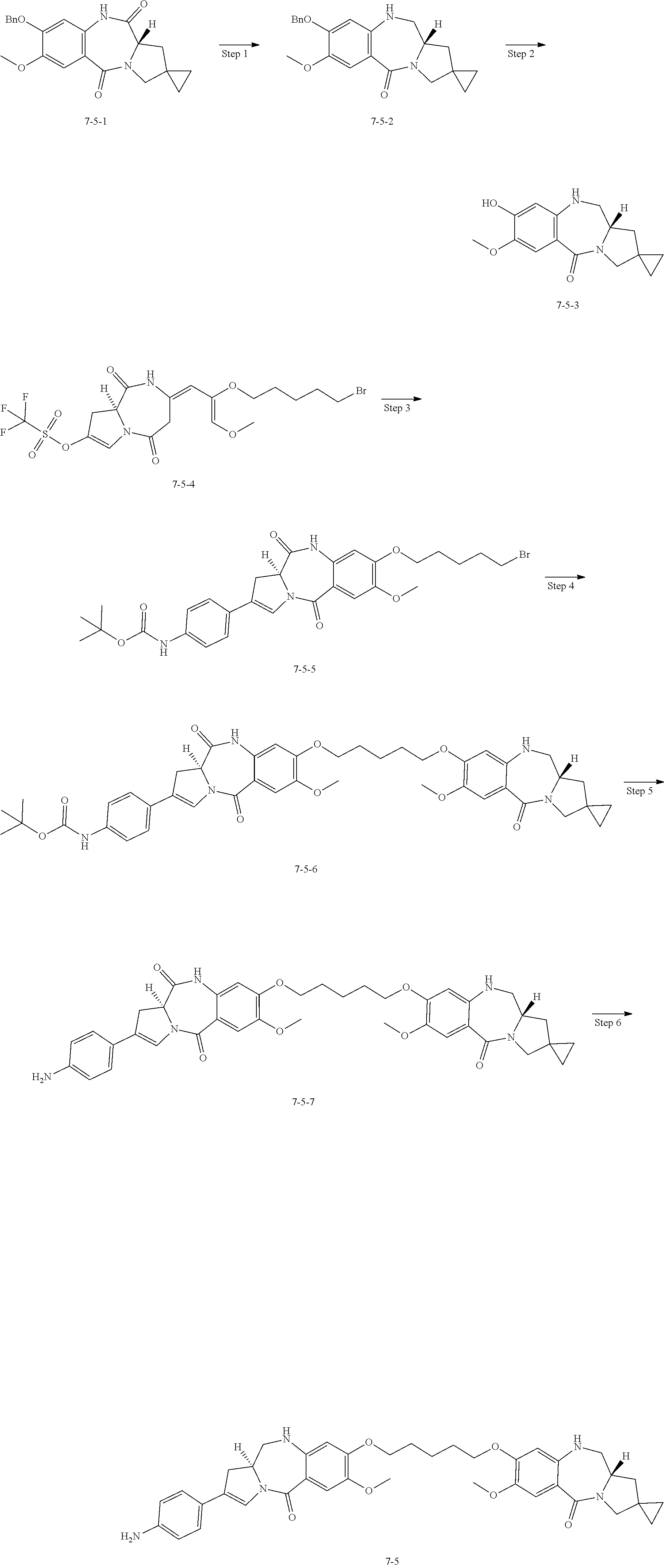

### Step 1: Preparation of (S)-8-(benzyloxy)-7-methoxy-1,10,11,11a-tetrahydro-3H,5H-spiro[benzo[e]pyrrolo[1,2-a][1,4]diazepin-2,1′-cyclopropane]-5-one (7-5-2)

(S)-8-(Benzyloxy)-7-methoxy-1,11a-dihydro-3H,5H-spiro[benzo[e]pyrrolo[1,2-a][1,4]diazepin-2,1′-cyclopropane]-5,11(10H)-dione (2 g, 5.29 mmol, FR) was dissolved in tetrahydrofuran (100 mL), followed by an addition of sodium borohydride (599.85 mg, 15.86 mmol), then trifluoroacetic acid (1.21 g, 10.57 mmol) was added dropwise at room temperature., until no large amount of gas overflowed, and then heated to 75° C. for reflux and reacted for 5 hours.

The reaction mixture was concentrated, followed by an addition of water (50 mL), and then extracted twice with ethyl acetate (50 mL×2). The organic phases were combined and washed with saturated sodium chloride aqueous solution, then dried over anhydrous sodium sulfate, filtered and concentrated to obtain the crude product of the title compound (1.82 g, 4.99 mmol), which was used directly in the next step without purification.

Its structural characterization data are as follows:

ESI-MS (m/z): 365.2[M+H]⁺

### Step 2: Preparation of (S)-8-hydroxy-7-methoxy-1,10,11,11a-tetrahydro-3H,5H-spiro[benzo[e]pyrrolo[1,2-a][1,4]diazepin-2,1′-cyclopropane]-5-one (7-5-3)

(S)-8-(Benzyloxy)-7-methoxy-1,10,11,11a-tetrahydro-3H,5H-spiro[benzo[e]pyrrolo[1,2-a][1,4]diazepin-2,1′-cyclopropane]-5-one (100 mg, 0.274 mmol, FR) was dissolved in dichloromethane (0.8 mL) and cooled to 0° C., followed by a dropwise addition of methanesulfonic acid (0.4 mL). After the addition was completed, the reaction mixture was reacted under thermal insulation conditions for 1 hour. Water (2 ml) was added to the reaction mixture, which was adjusted to pH=8 with saturated sodium bicarbonate aqueous solution and extracted with dichloromethane (3 mL×5) for 5 times. The combined organic phases were washed with saturated sodium chloride aqueous solution (3 mL), dried over anhydrous sodium sulfate, filtered and concentrated to obtain a crude product of the title compound (75 mg, 0.273 mmol), which was used directly in the next step without purification.

Its structural characterization data are as follows:

ESI-MS (m/z): 275.2[M+H]⁺

### Step 3: Preparation of tert-butyl (S)-(4-(8-((5-bromopentyl)oxy)-7-methoxy-5,11-dioxo-5,10,11,11a-tetrahydro-1H-benzo[e]pyrrolo[1,2-a][1,4]diazepin-2-yl)phenyl)carbamate (7-5-5)

(S)-8-((5-Bromopentyl)oxy)-7-methoxy-5,11-dioxo-5,10,11,11a-tetrahydro-1H-benzo[e]pyrrolo[1,2-a][1,4]diazepin-2-yl-trifluoromethanesulfonate (550 mg, 0.987 mmol), 4-(tert-butoxycarbonyl)aminophenylboronic acid (468 mg, 1.97 mmol) and sodium carbonate (418 mg, 3.95 mmol) were dissolved in a mixed solvent of toluene (10 mL), ethanol (1 mL) and water (1 mL), followed by an addition of tetrakis(triphenylphosphine)palladium (114 mg, 0.099 mmol), subjected to nitrogen replacement twice, heated to 80° C. and reacted for 3 hours under nitrogen protection. The reaction mixture was added to ethyl acetate (20 mL) and water (10 mL), stirred, and filtered. The filtrate was allowed to stand for separation. The organic phase was dried over anhydrous sodium sulfate, filtered and concentrated to obtain a crude product, which was purified by silica gel column (ethyl acetate/petroleum ether=0% to 50%) and concentrated again to obtain the title compound (397 mg, 0.661 mmol).

Its structural characterization data are as follows:

ESI-MS (m/z): 600.7[M+H]⁺

### Step 4: Preparation of tert-butyl (4-((S)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-5,11-dioxo-5,10,11,11a-tetrahydro-1-benzo[e]pyrrolo[1,2-a][1,4]diazepin-2-yl)phenyl)carbamate (7-5-6)

Tert-butyl (S)-(4-(8-((5-bromopentyl)oxy)-7-methoxy-5,11-dioxy-5,10,11,11a-tetrahydro-1H-benzo[e]pyrrolo[1,2-a][1,4]diazepin-2-yl)phenyl)carbamate (100 mg, 0.167 mmol) and (S)-8-hydroxy-7-methoxy-1,10,11,11a-tetrahydro-3H,5H-spiro[benzo[e]pyrrolo[1,2-a][1,4]diazepin-2,1′-cyclopropane]-5-one (46 mg, 0.167 mmol) were dissolved in DMF (1 mL), followed by an addition of potassium carbonate (69 mg, 0.500 mmol), and reacted under stirring for 16 hours. The reaction was quenched with water, and the mixture was extracted with ethyl acetate for 3 times (10 mL×3). The organic phases were combined, washed with saturated brine, dried and concentrated, and purified by silica gel column (MeOH/DCM=0% to 5%) to obtain the title compound (130 mg, 0.165 mmol)

Its structural characterization data are as follows:

ESI-MS (m/z): 794.4[M+H]⁺

### Step 5: Preparation of (S)-2-(4-aminophenyl)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (7-5-7)

Tert-butyl (4-((S)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-5,11-dioxo-5,10,11,11a-tetrahydro-1-benzo[e]pyrrolo[1,2-a][1,4]diazepin-2-yl)phenyl)carbamate (200 mg, 0.122 mmol) was dissolved in dichloromethane (2 mL), followed by a dropwise addition of trifluoroacetic acid (0.5 mL) under stirring. After the addition was completed, the reaction mixture was stirred continuously for 1 hour, then concentrated under reduced pressure, purified by reverse phase column chromatography (acetonitrile/l % formic acid aqueous solution=0% to 70%) and freeze-dried to obtain the title compound (31 mg, 0.045 mmol).

Its structural characterization data are as follows:

ESI-MS (m/z): 694.4 [M+H]⁺

### Step 6: Preparation of (S)-8-((5-(((S)-2-(4-aminophenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11-tetrahydro-3H,5H-spiro[benzo[e]pyrrolo[1,2-a][1,4]diazepin-2,1′-cyclopropane]-5-one (7-5)

(S)-2-(4-Aminophenyl)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (31 mg, 0.045 mmol) was dissolved in dry tetrahydrofuran (5 mL), followed by an addition of sodium borohydride (8.5 mg, 0.224 mmol) and a slow, dropwise addition of trifluoroacetic acid (20.4 mg, 0.179 mmol) to generate a large amount of gas. After the gas dissipated, the reaction mixture was slowly heated to 70° C. and reacted under refluxing for 16 hours. The reaction mixture was cooled to room temperature, followed by a dropwise addition of methanol to quench the reaction, and concentrated under reduced pressure. The concentrate was purified by reverse phase column chromatography (acetonitrile/l % formic acid aqueous solution=0% to 50%) and freeze-dried to obtain the title compound (4.65 mg, 0.007 mmol).

Its structural characterization data are as follows:

ESI-MS (m/z): 680.4[M+H]⁺

¹H NMR (400 MHz, DMSO): δ 7.31 (d, J=13.4 Hz, 2H), 7.26 (s, 1H), 7.11 (d, J=8.4 Hz, 2H), 6.53 (d, J=8.5 Hz, 3H), 6.29 (d, J=8.1 Hz, 3H), 5.19 (s, 2H), 4.13 (d, J=4.4 Hz, 1H), 3.93 (dd, J=12.0, 6.1 Hz, 4H), 3.83 (d, J=7.6 Hz, 1H), 3.65 (d, J=7.5 Hz, 6H), 3.56-3.46 (m, 3H), 3.31-3.11 (m, 4H), 2.69 (dd, J=16.7, 3.9 Hz, 1H), 1.95 (dd, J=12.5, 7.3 Hz, 1H), 1.79 (dd, J=12.5, 7.4 Hz, 5H), 1.56 (d, J=6.8 Hz, 2H), 0.67-0.62 (m, 1H), 0.60-0.50 (m, 3H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 2: Preparation of (S)-8-((5-(((S)-2-(4-Hydroxyphenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-3)

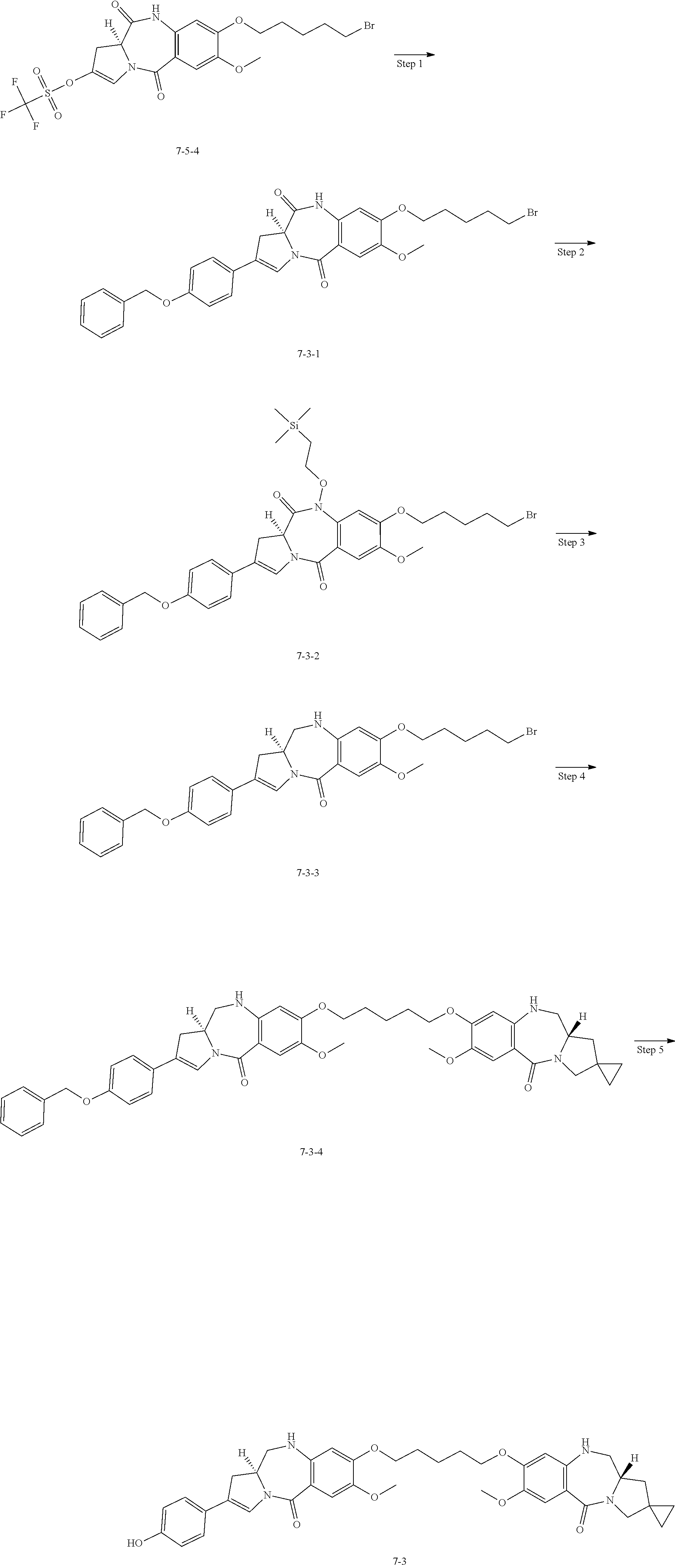 [SMILES: COc1cc2c(cc1OCCCCCBr)NC(=O)[C@@H]1CC(OS(=O)(=O)C(F)(F)F)=CN1C2=O ; COc1cc2c(cc1OCCCCCBr)NC(=O)[C@@H]1CC(c3ccc(OCc4ccccc4)cc3)=CN1C2=O ; COc1cc2c(cc1OCCCCCBr)N(OCC[Si](C)(C)C)C(=O)[C@@H]1CC(c3ccc(OCc4ccccc4)cc3)=CN1C2=O ; COc1cc2c(cc1OCCCCCBr)NC[C@@H]1CC(c3ccc(OCc4ccccc4)cc3)=CN1C2=O ; COc1cc2c(cc1OCCCCCOc1cc3c(cc1OC)C(=O)N1CC4(CC4)C[C@H]1CN3)NC[C@@H]1CC(c3ccc(OCc4ccccc4)cc3)=CN1C2=O ; COc1cc2c(cc1OCCCCCOc1cc3c(cc1OC)C(=O)N1CC4(CC4)C[C@H]1CN3)NC[C@@H]1CC(c3ccc(O)cc3)=CN1C2=O]

### Step 1: Preparation of (S)-2-(4-(benzyloxy)phenyl)-8-((5-bromopentyl)oxy)-7-methoxy-1,11a-dihydro-5H-benzo[e]pyrrolo[1,2-a][1,4]diazepin-5,11(10H)-dione (7-3-1)

(S)-8-((5-Bromopentyl)oxy)-7-methoxy-5,11-dioxo-5,10,11,11a-tetrahydro-1H-benzo[e]pyrrolo[1,2-a][1,4]diazepin-2-yl trifluoromethanesulfonate (500 mg, 897.13 μmol), (4-(benzyloxy)phenyl)boronic acid (409.18 mg, 1.79 mmol), tetrakis(triphenylphosphine) palladium (103.67 mg, 89.71 μmol) and sodium carbonate (380.34 mg, 3.59 mmol) were dissolved in toluene (8 mL), water (2 mL) and ethanol (2 mL). The reaction was carried out at 80° C. for 3 hours. After the reaction was completed, the reaction mixture was treated with water and ethyl acetate for extraction. The organic phases were combined, dried and concentrated to obtain a crude product. The crude product was purified by silica gel column chromatography to obtain the title compound (303 mg, 461.04 μmol).

Its structural characterization data are as follows:

ESI-MS (m/z): 591.4 [M+H]⁺

### Step 2: Preparation of (S)-2-(4-(benzyloxy)phenyl)-8-((5-bromopentyl)oxy)-7-methoxy-10-(2-(trimethylsilyl)ethoxy)methyl)-1,11a-dihydro-5H-benzo[e]pyrrolo[1,2-a][1,4]diazepin-5,11(10H)-dione (7-3-2)

(S)-2-(4-(Benzyloxy)phenyl)-8-((5-bromopentyl)oxy)-7-methoxy-1,11a-dihydro-5H-benzo[e]pyrrolo[1,2-a][1,4]diazepin-5,11(10H)-dione (186 mg, 314.46 μmol) was dissolved in tetrahydrofuran (5 mL) and cooled to −78° C. Then n-butyl lithium (294.81 μL, 471.69 μmol) was added dropwise. After the addition was completed, the reaction mixture was reacted under thermal insulation conditions for 0.5 hours. 2-(Trimethylsilyl)ethoxymethyl chloride (78.64 mg, 471.69 μmol) was added to the reaction mixture. After the addition was completed, the reaction mixture was continued at the same temperature for 1 hour. After the reaction was completed, the reaction was quenched with saturated ammonium chloride, and then the reaction mixture was extracted with ethyl acetate. The organic phase was dried and concentrated to obtain a crude product. The crude product was purified by silica gel column chromatography to obtain the title compound (200 mg, 263.25 μmol).

Its structural characterization data are as follows:

ESI-MS (m/z): 721.3 [M+H]⁺

### Step 3: Preparation of (S)-2-(4-(benzyloxy)phenyl)-8-((5-bromopentyl)oxy)-7-methoxy-1,10,11,11a-tetrahydro-5H-benzo[e]pyrrolo[1,2-a][1,4]diazepin-5-one (7-3-3)

Sodium borohydride (52.42 mg, 1.39 mmol) and calcium chloride (153.77 mg, 1.39 mmol) were dissolved in a mixture of tetrahydrofuran (2 mL) and ethanol (2 mL). Then, a solution of (S)-2-(4-(benzyloxy)phenyl)-8-((5-bromopentyl)oxy)-7-methoxy-10-(2-(trimethylsilyl)ethoxy)methyl)-1,11a-dihydro-5H-benzo[e]pyrrolo[1,2-a][1,4]diazepin-5,11(10H)-dione (100 mg, 138.55 μmol) in tetrahydrofuran (2 mL) was slowly added dropwise to the above system. The reaction was carried out at room temperature for 16 hours. After the reaction was completed, formic acid (0.5 mL) was added to quench the reaction, and the reaction mixture was stirred continuously for 10 minutes. After the detection indicated that the intermediate state conversion was completed, the reaction mixture was purified by preparative HPLC to obtain the title compound (14 mg, 23.03 μmol).

Its structural characterization data are as follows:

ESI-MS (m/z): 577.3 [M+H]⁺

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 60 | 40 | 28 |
| 2.00 | 60 | 40 | 28 |
| 18.00 | 90 | 10 | 28 |

### Step 4: Preparation of (S)-8-((5-(((S)-2-(4-(benzyloxy)phenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-3-4)

(S)-2-(4-(Benzyloxy)phenyl)-8-((5-bromopentyl)oxy)-7-methoxy-1,10,11,11a-tetrahydro-5H-benzo[e]pyrrolo[1,2-a][1,4]diazepin-5-one (12 mg, 20.78 μmol) and (S)-8-hydroxy-7-methoxy-1,10,11,11a-tetrahydro-3H,5H-spiro[benzo[e]pyrrolo[1,2-a][1,4]diazepin-2,1′-cyclopropane]-5-one (5.70 mg, 20.78 μmol) were dissolved in DMF (2 mL), followed by an addition of potassium carbonate (8.62 mg, 62.34 μmol), and reacted at room temperature for 4 hours. After the reaction was completed, the reaction was quenched with water, and the reaction mixture was extracted with ethyl acetate. The organic phases were combined, washed with saturated brine, dried and concentrated to obtain a crude product, which was purified by silica gel column chromatography to obtain the title compound (10 mg, 12.32 μmol).

Its structural characterization data are as follows:

ESI-MS (m/z): 771.4 [M+H]⁺

### Step 5: Preparation of (S)-8-((5-(((S)-2-(4-hydroxyphenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-3)

(S)-8-((5-(((S)-2-(4-(Benzyloxy)phenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (10.00 mg, 12.97 μmol) was dissolved in dichloromethane (2 mL), followed by a dropwise addition of methanesulfonic acid (0.5 mL) under stirring. After the addition was completed, the reaction mixture was stirred continuously for 16 hours. After the reaction was completed, the reaction mixture was purified and freeze-dried to obtain the title compound (0.47 mg, 0.69 μmol).

Its structural characterization data are as follows:

ESI-MS (m/z): 681.3 [M+H]⁺

¹H NMR (400 MHz, DMSO): δ ¹H NMR (400 MHz, DMSO) δ 9.49 (s, 1H), 7.37 (s, 1H), 7.32 (s, 1H), 7.29 (s, 1H), 7.26 (d, J=8.8 Hz, 2H), 6.73 (d, J=8.4 Hz, 2H), 6.54 (d, J=6.4 Hz, 1H), 6.31-6.28 (m, 3H), 4.20-4.12 (m, 1H), 3.95-3.89 (m, 3H), 3.86-3.81 (m, 1H), 3.66 (s, 3H), 3.64 (s, 3H), 3.57-3.46 (m, 3H), 3.30-3.28 (m, 1H), 3.26-3.20 (m, 1H), 3.17-3.11 (m, 1H), 2.76-2.67 (m, 2H), 1.98-1.93 (m, 1H), 1.83-1.77 (m, 4H), 1.59-1.52 (m, 2H), 0.67-0.62 (m, 1H), 0.60-0.56 (m, 1H), 0.56-0.50 (m, 2H).

Its preparation method was as follows:
Chromatographic column: SunFire Prep C18 OBD 19 mm×150 mm×5.0 μm
Mobile phase A: acetonitrile; Mobile phase B: water (0.05% ammonium bicarbonate)

| Time [min] | Mobile phase A [%] | Mobile phase B [%] | Flow rate [mL/min] |
|---|---|---|---|
| 0.00 | 10 | 90 | 28 |
| 2.00 | 10 | 90 | 28 |
| 18.00 | 80 | 20 | 28 |

### Preparation Example 3: Preparation of (11aS,11a″S)-8,8″-(pentan-1,5-diylbis(oxy))bis(7-methoxy-1,10,11,11a-tetrahydro-3H,5H-spiro[benzo][e]pyrrolo[1,2-a][1,4]diazepin-2,1′-cyclopropane]-5-one) (7-9)

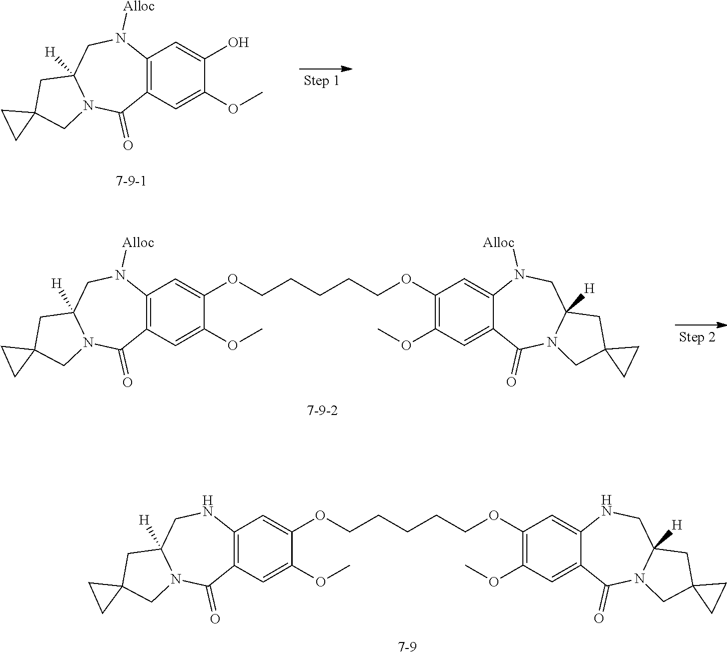

### Step 1: Preparation of 8,8″-(pentan-1,5-diylbis(oxy))(11aS,11a″S)-bis(7-methoxy-5-oxo-11,11a-dihydro-1H,3H-spiro[benzo[e]pyrrolo[1,2-a][1,4]diazepin-2,1′-cyclopropane]-10(5H)-carboxylic acid diallyl ester) (7-9-2)

Allyl (S)-8-hydroxy-7-methoxy-5-oxo-11,11a-dihydro-1H,3H-spiro[benzo[e]pyrrolo[1,2-a][1,4]diazepin-2,1′-cyclopropane]-10(5H)-carboxylate (55 mg, 153.47 μmol) and 1,5-dibromopentane (17.64 mg, 76.73 μmol) was added to DMF (2 mL), followed by an addition of potassium carbonate (63.63 mg, 460.40 μmol), and reacted at room temperature for 20 hours. After the reaction was completed, 10 mL of water was added to the reaction mixture, and the mixture was extracted with ethyl acetate 3 times (5 mL×3). The organic phases were combined and washed with 5 mL of saturated brine, dried over anhydrous sodium sulfate, and concentrated under reduced pressure. The resultant crude product was purified by preparative thin layer chromatography (petroleum ether/ethyl acetate=1:2) and concentrated under reduced pressure again to obtain the title compound (100 mg, 127.41 μmol).

Its structural characterization data are as follows:

ESI-MS (m/z): 785.4[M+H]⁺

### Step 2: Preparation of (11aS,11a″S)-8,8″-(pentane-1,5-diylbis(oxy))bis(7-methoxy-1,10,11,11a-tetrahydro-3H,5H-spiro[benzo][e]pyrrolo[1,2-a][1,4]diazepin-2,1′-cyclopropane]-5-one) (7-9)

8,8″-(Pentan-1,5-diylbis(oxy))(11aS,11a″S)-bis(7-methoxy-5-oxo-11,11a-dihydro-1H,3H-spiro[benzo[e]pyrrolo[1,2-a][1,4]diazepin-2,1′-cyclopropane]-10(5H)-carboxylic acid diallyl ester) (80 mg, 101.92 μmol) and tetrahydropyrrole (14.50 mg, 203.85 μmol) were dissolved in DMF (1.5 mL), followed by an addition of Pd(PPh₃)₄ (11.78 mg, 10.19 μmol) under nitrogen protection, subjected to nitrogen replacement for three times, and then reacted under stirring and nitrogen protection at room temperature for 2 hours. After the reaction was completed, 5 mL of water was added to the reaction mixture, and the mixture was extracted with 5 mL of ethyl acetate (5 mL×3). The organic phase was washed with 5 mL of saturated brine, dried over anhydrous sodium sulfate and concentrated under reduced pressure. The resultant crude product was first purified by preparative thin layer chromatography (ethyl acetate:methanol=20:1) to remove triphenylphosphine and then concentrated under reduced pressure again, purified by preparative HPLC again and freeze-dried to obtain the title compound (12.83 mg, 20.18 μmol).

Its structural characterization data are as follows:

ESI-MS (m/z): 617.4 [M+H]⁺; 309.3 [M/2+1]⁺

¹H NMR (400 MHz, CDCl₃) δ 7.54 (s, 2H), 6.10 (s, 2H), 3.99 (dd, J=16.0, 10.4 Hz, 3H), 3.88 (d, J=30.0 Hz, 3H), 3.74-3.38 (m, 4H), 2.04 (dd, J=17.2, 7.6 Hz, 1H), 1.96-1.83 (m, 2H), 1.71 (ddd, J=21.6, 12.0, 5.6 Hz, 2H), 0.77-0.66 (m, 1H), 0.66-0.50 (m, 3H).

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 10 | 90 | 28 |
| 2.00 | 10 | 90 | 28 |
| 16.00 | 90 | 10 | 28 |

### Preparation Example 4: Preparation of (S)-8-((5-(((S)-2-(4-aminophenyl)-7-methoxy-5-oxo-5,10,11,11a-tetrahydro-1H-benzo[e]pyrrolo[1,2-a][1,4]diazepin-8-yl)oxy)pentyl)oxy)-7-(methoxy-d3)-1,10,11,11a-tetrahydro-3H,5H-spiro[benzo[e]pyrrolo[1,2-a][1,4]diazepin-2,1′-cyclopropane]-5-one (7-10)

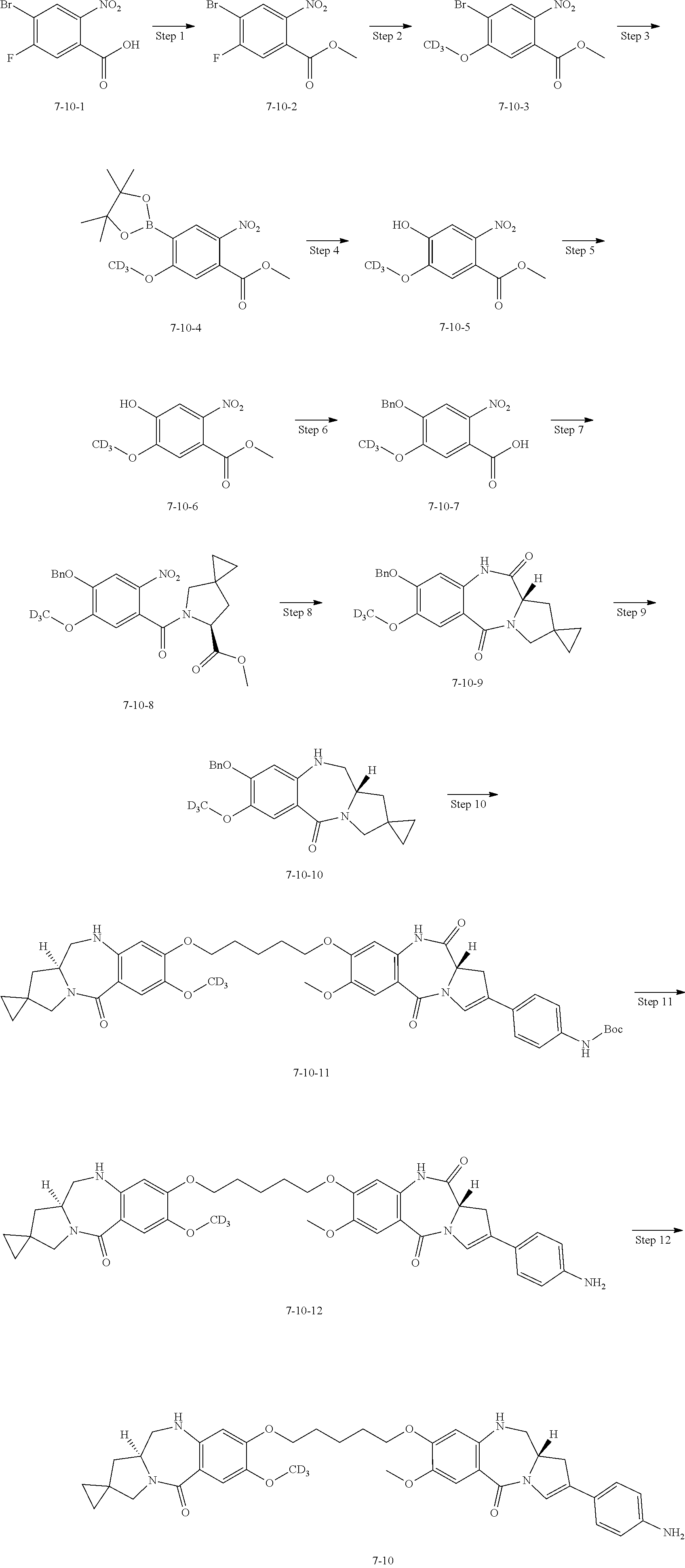

### Step 1: Preparation of methyl 4-bromo-5-fluoro-2-nitrobenzoate (7-10-2)

4-Bromo-5-fluoro-2-nitrobenzoic acid (5.9 g, 22.35 mmol, FR) was dissolved in methanol (30 mL), followed by an addition of thionyl chloride (30 mL), and reacted at room temperature for 16 hours. The reaction mixture was concentrated to obtain a crude product, which was purified by a silica gel column (ethyl acetate/petroleum ether=0% to 50%) and concentrated again to obtain the title compound (3.05 g, 10.97 mmol).

### Step 2: Preparation of methyl 4-bromo-5-(methoxy-d3)-2-nitrobenzoate (7-10-3)

Methyl 4-bromo-5-fluoro-2-nitrobenzoate (1.5 g, 5.4 mmol, FR) was dissolved in DMF (6 mL), followed by an addition of deuterated methanol (388.5 mg, 10.8 mmol) and cesium carbonate (2.64 mg, 8.09 mmol), subjected to nitrogen replacement for 3 times, and reacted at room temperature for 16 hours. Ethyl acetate (20 mL) and water (30 mL) were added to the reaction mixture. The resulting mixture was stirred and allowed to stand for separation. The organic phase was dried over anhydrous sodium sulfate, filtered and concentrated to obtain a crude product, which was purified by silica gel column (ethyl acetate/petroleum ether=0% to 50%) and concentrated to obtain the title compound (1.08 g, 3.68 mmol).

Its structural characterization data are as follows:

¹H NMR (400 MHz, DMSO): δ 8.35 (s, 1H), 7.51 (s, 1H).

### Step 3: Preparation of methyl 5-(methoxy-d3)-2-nitro-4-(4,4,5,5-tetramethyl-1,3,2-dioxaborolan-2-yl)benzoate (7-10-4)

Methyl 4-bromo-5-(methoxy-d3)-2-nitrobenzoate (1.08 g, 3.68 mmol), 4,4,4′,4′,5,5,5′,5′-octamethyl-2,2′-bis(1,3,2-dioxaborolan) (2.34 g, 9.21 mmol), bis(triphenylphosphine)palladium dichloride (775.94 mg, 1.11 mmol) and potassium acetate (1.08 g, 11.05 mmol) were added to a single-necked bottle, followed by an addition of 1,4-dioxane (15 mL), subjected to nitrogen replacement for 3 times, heated to 101° C. and reacted for 16 hours. The reaction mixture was then cooled to room temperature, filtered through diatomaceous earth and concentrated to obtain a crude product (1.1 g, 3.23 mmol), which was used directly in the next step without purification.

### Step 4: Preparation of methyl 4-hydroxy-5-(methoxy-d3)-2-nitrobenzoate (7-10-5)

Methyl 5-(methoxy-d3)-2-nitro-4-(4,4,5,5-tetramethyl-1,3,2-dioxaborolan-2-yl)benzoate (1.1 g, 3.23 mmol) was dissolved in tetrahydrofuran (30 mL), followed by an addition of glacial acetic acid (3.75 mL) at 0° C. and a slow, dropwise addition of hydrogen peroxide (7 mL), and reacted at room temperature for 3 hours. Saturated sodium sulfite solution (30 mL) was added to the reaction mixture, then the resulting mixture was stirred for 1 hour and concentrated under reduced pressure until no tetrahydrofuran remained, followed by extraction with 30 mL of ethyl acetate, and allowed to liquid separation. The organic phase was dried over anhydrous sodium sulfate, filtered and concentrated to obtain a crude product, which was purified by silica gel column (ethyl acetate/petroleum ether=0% to 70%) and concentrated to obtain the title compound (680 mg, 2.95 mmol).

Its structural characterization data are as follows:

ESI-MS (m/z): 230.2 [M−H]⁻

### Step 5: Preparation of methyl 4-(benzyloxy)-5-(methoxy-d3)-2-nitrobenzoate (7-10-6)

Methyl 4-hydroxy-5-(methoxy-d3)-2-nitrobenzoate (680 mg, 2.95 mmol) was dissolved in DMF (5 mL), followed by an addition of potassium carbonate (815.3 mg, 5.91 mmol) and benzyl bromide (1.01 g, 5.91 mmol), and reacted at room temperature for 15 hours. 30 mL of water was added to the reaction solution, then 30 mL of ethyl acetate was added for extraction, the resulting mixture was allowed to stand for liquid separation. The organic phase was dried over anhydrous sodium sulfate, filtered and concentrated to obtain a crude product, which was purified by silica gel column (ethyl acetate/petroleum ether=0% to 50%) and concentrated to obtain the title compound (688 mg, 2.15 mmol).

### Step 6: Preparation of 4-(benzyloxy)-5-(methoxy-d3)-2-nitrobenzoic Acid (7-10-7)

Methyl 4-(benzyloxy)-5-(methoxy-d3)-2-nitrobenzoate (688 mg, 2.15 mmol) was dissolved in tetrahydrofuran (5 mL) and water (1 mL), followed by an addition of lithium hydroxide (103.1 mg, 4.3 mmol), and reacted at room temperature for 3 hours. The system was adjusted to pH=5 with dilute hydrochloric acid to precipitate solids, then 30 mL of ethyl acetate was added for extraction, and the resulting mixture was allowed to stand for liquid separation. The organic phase was washed with saturated brine, dried over anhydrous sodium sulfate, filtered, and concentrated to obtain the title compound (400 mg, 1.31 mmol).

### Step 7: Preparation of methyl (S)-5-(4-(benzyloxy)-5-(methoxy-d3)-2-nitrobenzoyl)-5-azaspiro[2.4]heptan-6-carboxylate (7-10-8)

4-(Benzyloxy)-5-(methoxy-d3)-2-nitrobenzoic acid (400 mg, 1.31 mmol) and methyl (S)-5-azaspiro[2.4]heptan-6-carboxylate (203 mg, 1.31 mmol) were dissolved in DMF (5 mL), followed by an addition of HATU (597.3 mg, 1.57 mmol) and DIPEA (507 mg, 3.93 mmol), and reacted at room temperature for 15 hours. 30 mL of water was added to the reaction mixture, then 30 mL of ethyl acetate was added for extraction. After liquid separation, the organic phase was dried over anhydrous sodium sulfate, filtered and concentrated to obtain a crude product, which was purified by silica gel column (ethyl acetate/petroleum ether=0% to 50%) and concentrated to obtain the title compound (570 mg, 1.29 mmol).

Its structural characterization data are as follows:

ESI-MS (m/z): 444.1 [M+H]⁺

### Step 8: Preparation of (S)-8-(benzyloxy)-7-(methoxy-d3)-1,11a-dihydro-3H,5H-spiro[benzo[e]pyrrolo[1,2-a][1,4]diazepin-2,1′-cyclopropane]-5,11(10H)-dione (7-10-9)

Methyl (S)-5-(4-(benzyloxy)-5-(methoxy-d3)-2-nitrobenzoyl)-5-azaspiro[2.4]heptan-6-carboxylate (570 mg, 1.29 mmol) was dissolved in methanol (30 mL), followed by an addition of 10% aqueous ammonium chloride solution (4 mL) and zinc powder (1.28 g, 19.51 mmol), heated to 40° C. and reacted for 1 hour, then heated to 80° C. and reacted overnight, and concentrated to obtain a crude product, 35 mL ethyl acetate was added to the crude product for dissolution and washed with saturated brine 3 times. The organic phase was dried over anhydrous sodium sulfate, filtered and concentrated to obtain a crude product, which was purified by reverse phase column chromatography (acetonitrile/1% formic acid aqueous solution=0% to 70%) and freeze-dried to obtain the title compound (306 mg, 0.8 mmol).

Its structural characterization data are as follows:

ESI-MS (m/z): 382.2[M+H]⁺

### Step 9: Preparation of (S)-8-hydroxy-7-(methoxy-d3)-1,11a-dihydro-3H,5H-spiro[benzo[e]pyrrolo[1,2-a][1,4]diazepin-2,1′-cyclopropane]-5,11(10H)-dione (7-10-10)

(S)-8-(Benzyloxy)-7-(methoxy-d3)-1,11a-dihydro-3H,5H-spiro[benzo[e]pyrrolo[1,2-a][1,4]diazepin-2,1′-cyclopropane]-5,11(10H)-dione (132 mg, 0.29 mmol) was dissolved in dichloromethane (1 mL), cooled to 0° C., followed by the dropwise addition of methanesulfonic acid (0.5 mL). After the addition was completed, the reaction was carried out under thermal insulation conditions for 1 hour. Water (3 ml) was added to the reaction mixture, which was adjusted to pH=8 with saturated sodium bicarbonate aqueous solution and extracted with dichloromethane (5 mL×3) 3 times. The organic phases were combined, washed with saturated sodium chloride aqueous solution (10 mL), dried over anhydrous sodium sulfate, filtered and concentrated to obtain a crude product of the title compound (100 mg, 0.27 mmol), which was used directly in the next step without purification.

Its structural characterization data are as follows:

ESI-MS (m/z): 292.1 [M+H]⁺

### Step 10: Preparation of tert-butyl (4-((S)-7-methoxy-8-((5-(((S)-7-(methoxy-d3))-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-5,11-dioxo-5,10,11,11a-tetrahydro-1H-benzo[e]pyrrolo[1,2-a][1,4]diazepin-2-yl)phenyl)carbamate (7-10-11)

Tert-butyl (S)-(4-(8-((5-bromopentyl)oxy)-7-methoxy-5,11-dioxy-50,11,11-tetrahydro-1H-benzo[e]pyrrolo[1,2-a][1,4]diazepin-2-yl)phenyl)carbamate (167 mg, 0.27 mmol) and (S)-8-hydroxy-7-(methoxy-d3)-1,11a-dihydro-3H,5H-spiro[benzo[e]pyrrolo[1,2-a][1,4]diazepin-2,1′-cyclopropane]-5,11(10H)-dione (100 mg, 0.27 mmol) were dissolved in DMF (2 mL), followed by an addition of potassium carbonate (60 mg, 0.54 mmol), and reacted under stirring for 16 hours. The reaction was quenched with water, and the reaction mixture was extracted with ethyl acetate 3 times (10 mL×3). The organic phases were then combined, washed with saturated brine, dried, concentrated, and purified by silica gel column (MeOH/DCM=0% to 5%) to obtain the title compound (120 mg, 0.136 mmol).

Its structural characterization data are as follows:

ESI-MS (m/z): 797.3 [M+H]⁺

### Step 11: Preparation of (S)-2-(4-aminophenyl)-7-methoxy-8-((5-(((S)-7-(methoxy-d3)-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (7-10-12)

Tert-butyl (4-((S)-7-methoxy-8-((5-(((S)-7-(methoxy-d3))-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-5,11-dioxo-5,10,11,11a-tetrahydro-1H-benzo[e]pyrrolo[1,2-a][1,4]diazepin-2-yl)phenyl)carbamate (120 mg, 0.136 mmol) was dissolved in dichloromethane (3 mL), followed by a dropwise addition of trifluoroacetic acid (0.6 mL) under stirring. After the addition was completed, the reaction mixture was stirred continuously for 1 hour, concentrated under reduced pressure, purified by reverse phase column chromatography (acetonitrile/1% formic acid aqueous solution=0% to 70%) and freeze-dried to obtain the title compound (35 mg, 0.05 mmol).

Its structural characterization data are as follows:

ESI-MS (m/z): 697.3 [M+H]⁺

### Step 12: Preparation of (S)-8-((5-(((S)-2-(4-aminophenyl)-7-methoxy-5-oxo-5,10,11,11a-tetrahydro-1H-benzo[e]pyrrolo[1,2-a][1,4]diazepin-8-yl)oxy)pentyl)oxy)-7-(methoxy-d3)-1,10,11,11a-tetrahydro-3H,5H-spiro[benzo[e]pyrrolo[1,2-a][1,4]diazepin-2,1′-cyclopropane]-5-one (7-10)

(S)-2-(4-Aminophenyl)-7-methoxy-8-((5-(((S)-7-(methoxy-d3)-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (35 mg, 0.05 mmol) was dissolved in dry tetrahydrofuran (5 mL), followed by an addition of sodium borohydride (8.8 mg, 0.225 mmol) and a slow, dropwise addition of trifluoroacetic acid (20.9 mg, 0.18 mmol) to generate a large amount of gas. After the gas was dissipated, the reaction mixture was slowly heated to 70° C. and reacted under refluxing for 16 hours. The reaction mixture was cooled to room temperature, and methanol was added dropwise to quench the reaction. The reaction mixture was concentrated under reduced pressure, purified by reverse phase column chromatography (acetonitrile/1% formic acid aqueous solution=0% to 50%) and freeze-dried to obtain the title compound (2.9 mg, 0.004 mmol).

Its structural characterization data are as follows:

ESI-MS (m/z): 683.3 [M+H]⁺

¹H NMR (400 MHz, DMSO): δ 7.33 (s, 1H), 7.30 (s, 1H), 7.27 (s, 1H), 7.13 (d, J=8.4 Hz, 1H), 6.58-6.53 (m, 2H), 6.34-6.26 (m, 2H), 4.20-4.10 (m, 1H), 4.0-3.92 (m, 4H), 3.86-3.80 (m, 2H), 3.67 (s, 3H), 3.56 (s, 3H), 3.3-3.22 (m, 3H), 3.20-3.12 (m, 2H), 2.76-2.66 (m, 2H), 2.02-1.94 (m, 1H), 1.85-1.77 (m, 4H), 1.63-1.52 (m, 2H), 0.62-0.52 (m, 3H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 25 |
| 2.00 | 30 | 90 | 25 |
| 24.00 | 90 | 10 | 25 |

### Preparation Example 5: Preparation of (S)-2-(4-aminophenyl)-8-((5-(((S)-7-cyclopropyloxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-7-methoxy-1,11a-dihydro-5H-benzo[e]pyrrolo[1,2-a][1,4]diazepin-5,11(10H)-dione (7-11)

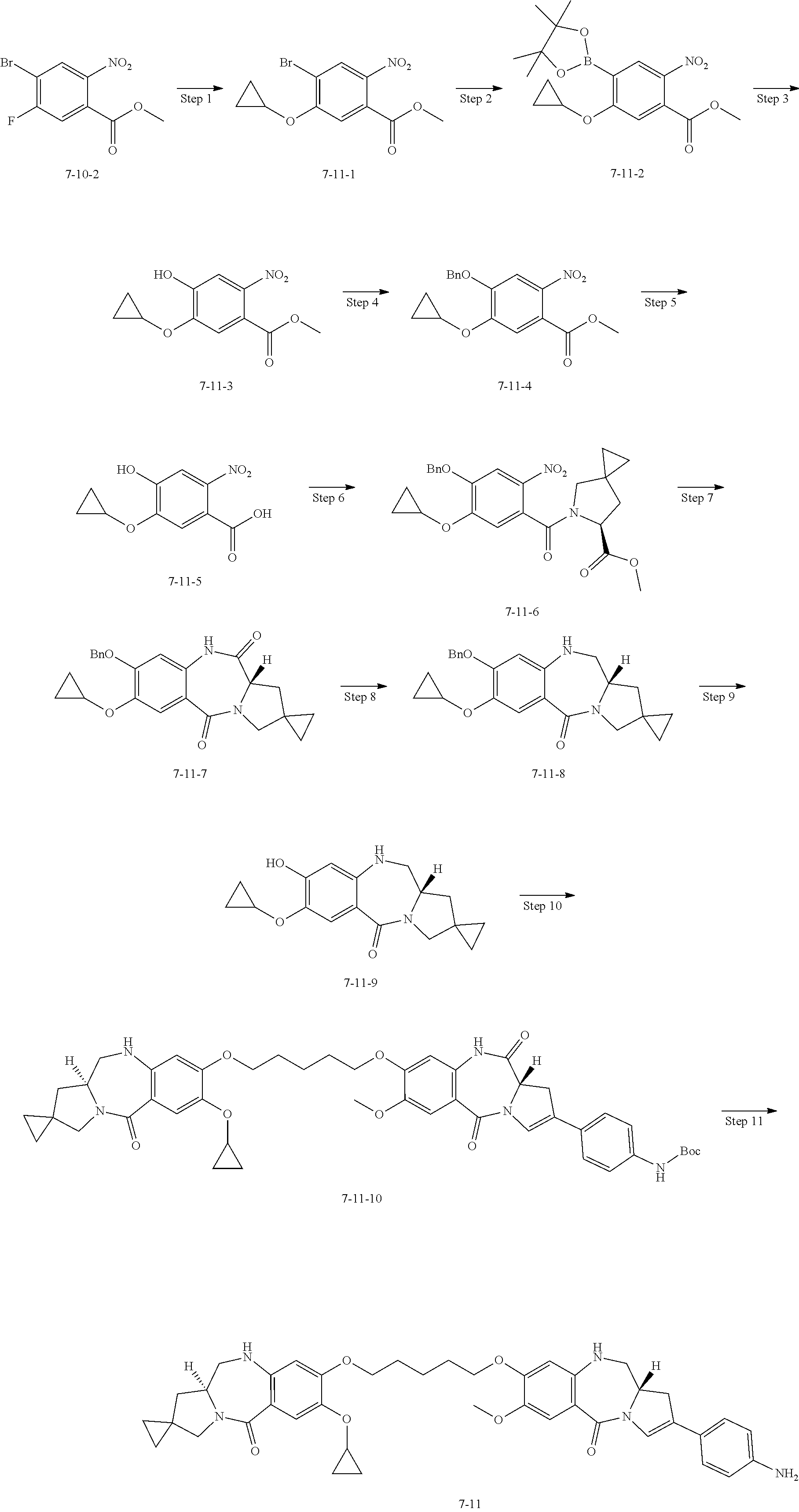

### Step 1: Preparation of methyl 4-bromo-5-cyclopropyloxy-2-nitrobenzoate (7-11-1)

Methyl 4-bromo-5-fluoro-2-nitrobenzoate (1.5 g, 5.4 mmol, FR) was dissolved in DMF (6 mL), followed by an addition of cyclopropanol (635 mg, 10.97 mmol) and cesium carbonate (2.68 mg, 8.3 mmol), subjected to nitrogen replacement for three times, and then reacted at room temperature for 16 hours. The reaction mixture was added to ethyl acetate (30 mL) and water (30 mL), stirred, and allowed to stand for liquid separation. The resultant organic phase was dried over anhydrous sodium sulfate, filtered and concentrated to obtain a crude product, which was purified by silica gel column (ethyl acetate/petroleum ether=0% to 50%) and concentrated to obtain the title compound (750 mg, 2.37 mmol).

Its structural characterization data are as follows:

¹H NMR (400 MHz, DMSO): δ 8.41 (s, 1H), 7.70 (s, 1H), 4.21 (tt, J=5.8, 2.8 Hz, 1H), 3.88 (s, 3H), 0.92 (m, 2H), 0.80 (m, 2H).

### Step 2: Preparation of methyl 5-cyclopropyloxy-2-nitro-4-(4,4,5,5-tetramethyl-1,3,2-dioxaborolan-2-yl)benzoate (7-11-2)

Methyl 4-bromo-5-cyclopropyloxy-2-nitrobenzoate (750 mg, 2.37 mmol), 4,4,4′,4′,5,5,5′,5′-octamethyl-2,2′-bis(1,3,2-dioxaborolane) (1.5 g, 5.93 mmol), bis(triphenylphosphine)palladium dichloride (499.6 mg, 0.71 mmol), and potassium acetate (700 mg, 7.12 mmol) were added to a single-necked bottle, followed by an addition of 1,4-dioxane (15 mL). The resulting mixture was subjected to nitrogen replacement for 3 times, heated to 101° C. and reacted for 16 hours, then cooled to room temperature, filtered through diatomaceous earth and concentrated to obtain a crude product (1.6 g, 4.41 mmol), which was used directly in the next step without purification.

### Step 3: Preparation of methyl 5-cyclopropyloxy-4-hydroxy-2-nitrobenzoate (7-11-3)

Methyl 5-cyclopropyloxy-2-nitro-4-(4,4,5,5-tetramethyl-1,3,2-dioxaborolan-2-yl)benzoate (1.6 g, 4.41 mmol) was dissolved in tetrahydrofuran (40 mL), followed by an addition of glacial acetic acid (5 mL) at 0° C. and a slow, dropwise addition of hydrogen peroxide (10 mL), and reacted at room temperature for 3 hours. The reaction mixture was treated with saturated sodium sulfite solution (50 mL) and stirred for 1 hour, then concentrated under reduced pressure until no tetrahydrofuran remained. 30 mL of ethyl acetate was added to the residue for extraction, and allowed to stand for separation of liquid layers. The organic phase was dried over anhydrous sodium sulfate, filtered and concentrated to obtain a crude product, which was purified by silica gel column (ethyl acetate/petroleum ether=0% to 70%) and concentrated to obtain the title compound (1.05 g, 4.15 mmol).

Its structural characterization data are as follows:

ESI-MS (m/z): 253.2 [M−H]⁻

### Step 4: Preparation of methyl 4-(benzyloxy)-5-cyclopropyloxy-2-nitrobenzoate (7-11-4)

Methyl 5-cyclopropyloxy-4-hydroxy-2-nitrobenzoate (1.05 g, 4.15 mmol) was dissolved in DMF (5 mL), followed by an addition of potassium carbonate (1.2 g, 8.6 mmol) and benzyl bromide (1.49 g, 8.6 mmol), and reacted at room temperature for 15 hours. 30 mL of water was added to the reaction mixture, followed by extraction with 50 mL of ethyl acetate. After liquid separation, the organic phase was dried over anhydrous sodium sulfate, filtered and concentrated to obtain a crude product, which was purified by silica gel column (ethyl acetate/petroleum ether=0% to 50%) and concentrated to obtain the title compound (1.09 g, 3.17 mmol).

### Step 5: Preparation of 4-(benzyloxy)-5-cyclopropyloxy-2-nitrobenzoic Acid (7-11-5)

Methyl 4-(benzyloxy)-5-cyclopropyloxy-2-nitrobenzoate (1.09 g, 3.17 mmol) was dissolved in tetrahydrofuran (8 mL) and water (2 mL), followed by an addition of lithium hydroxide (152.1 mg, 6.35 mmol), and reacted at room temperature for 3 hours. The system was adjusted to pH=5 with dilute hydrochloric acid to precipitate solids and extracted with 60 mL of ethyl acetate. After liquid separation, the organic phase was washed with saturated brine, dried over anhydrous sodium sulfate, filtered, and concentrated to obtain the title compound (743 mg, 2.26 mmol), which was used directly in the next step without purification.

### Step 6: Preparation of methyl (S)-5-(4-(benzyloxy)-5-cyclopropyloxy-2-nitrobenzoyl)-5-azaspiro[2.4]heptan-6-carboxylate (7-11-6)

4-(Benzyloxy)-5-cyclopropyloxy-2-nitrobenzoic acid (743 mg, 2.26 mmol) and methyl (S)-5-azaspiro[2.4]heptan-6-carboxylate (700 mg, 4.51 mmol) were dissolved in DMF (5 mL), followed by an addition of HATU (1.71 g, 4.51 mmol) and DIPEA (875 mg, 6.77 mmol), and reacted at room temperature for 2 hours. 30 mL of water was added to the reaction mixture, followed by extraction with 50 mL of ethyl acetate. After liquid separation, the organic phase was dried over anhydrous sodium sulfate, filtered and concentrated to obtain a crude product, which was purified by silica gel column (ethyl acetate/petroleum ether=0% to 50%) and concentrated to obtain the title compound (910 mg, 1.95 mmol).

Its structural characterization data are as follows:

ESI-MS (m/z): 467.1 [M+H]⁺

### Step 7: Preparation of (S)-8-(benzyloxy)-7-cyclopropyloxy-1,11a-dihydro-3H,5H-spiro[benzo[e]pyrrolo[1,2-a][1,4]diazepin-2,1′-cyclopropane]-5,11(10H)-dione (7-11-7)

Methyl (S)-5-(4-(Benzyloxy)-5-cyclopropyloxy-2-nitrobenzoyl)-5-azaspiro[2.4]heptan-6-carboxylate (910 mg, 1.95 mmol) was dissolved in methanol (30 mL), followed by an addition of 10% aqueous ammonium chloride solution (6 mL) and zinc powder (1.28 g, 19.51 mmol), heated to 40° C. and reacted for 1 hour, then heated to 80° C. and reacted overnight. The reaction mixture was concentrated to obtain a crude product, 35 mL ethyl acetate was added to the crude product for dissolution, and washed 3 times with saturated brine. The organic phase was dried over anhydrous sodium sulfate, filtered and concentrated to obtain a crude product, which was purified by reverse phase column chromatography (acetonitrile/l % formic acid aqueous solution=0% to 70%) and freeze-dried to obtain the title compound (467 mg, 1.15 mmol).

Its structural characterization data are as follows:

ESI-MS (m/z): 405.2 [M+H]⁺

### Step 8: Preparation of (S)-8-(benzyloxy)-7-cyclopropyloxy-1,10,11,11a-tetrahydro-3H,5H-spiro[benzo[e]pyrrolo[1,2-a][1,4]diazepin-21,1′-cyclopropane]-5-one (7-11-8)

(S)-8-(Benzyloxy)-7-cyclopropyloxy-1,11a-dihydro-3H,5H-spiro[benzo[e]pyrrolo[1,2-a][1,4]diazepin-2,1′-cyclopropane]-5,11(10H)-dione (467 mg, 1.15 mmol) was dissolved in dry tetrahydrofuran (20 mL), followed by an addition of sodium borohydride (128 mg, 3.46 mmol) and a slow, dropwise addition of trifluoroacetic acid (264 mg, 2.31 mmol) to generate a large amount of gas. After the gas was dissipated, the reaction mixture was slowly heated to 70° C. and reacted under refluxing for 16 hours. The reaction mixture was cooled to room temperature, followed by a dropwise addition of methanol to quench the reaction, concentrated under reduced pressure, and purified by reverse phase column chromatography (acetonitrile/1% formic acid aqueous solution=0% to 80%) and freeze-dried to obtain the title compound (380 mg, 0.97 mmol).

Its structural characterization data are as follows:

ESI-MS (m/z): 363.3 [M+H]⁺

### Step 9: Preparation of (S)-7-cyclopropyloxy-8-hydroxy-1,10,11,11a-tetrahydro-3H,5H-spiro[benzo[e]pyrrolo[1,2-a][1,4]diazepin-2,1′-cyclopropane]-5-one (7-11-9)

(S)-8-(Benzyloxy)-7-cyclopropyloxy-1,10,11,11a-tetrahydro-3H,5H-spiro[benzo[e]pyrrolo[1,2-a][1,4]diazepin-2,1′-cyclopropane]-5-one (380 mg, 0.97 mmol) was dissolved in dichloromethane (1.5 mL), cooled to 0° C., followed by a dropwise addition of methanesulfonic acid (0.6 mL). After the addition was completed, the reaction was carried out under thermal insulation conditions for 1 hour. Water (10 ml) was added to the reaction solution, then adjusted to pH=8 with saturated sodium bicarbonate aqueous solution, and extracted with dichloromethane (8 mL×3) three times. The organic phases were combined and washed with saturated sodium chloride aqueous solution (20 mL), dried over anhydrous sodium sulfate, filtered and concentrated to obtain a crude product of the title compound (118 mg, 0.31 mmol), which was used directly in the next step without purification.

Its structural characterization data are as follows:

ESI-MS (m/z): 301.1 [M+H]⁺

### Step 10: Preparation of tert-butyl (4-((S)-8-((5-(((S)-7-cyclopropyloxy-5-oxo-5,10,11,11a-tetrahydro-1H,3H-spiro[benzo[e]]pyrrolo[1,2-a][1,4]diazepin-2,1′-cyclopropane]-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (7-11-10)

Tert-butyl (S)-(4-(8-((5-bromopentyl)oxy)-7-methoxy-5,11-dioxy-50,11,11-tetrahydro-1H-benzo[e]pyrrolo[1,2-a][1,4]diazepin-2-yl)phenyl)carbamate (141 mg, 0.23 mmol) and (S)-7-cyclopropyloxy-8-hydroxy-1,10,11,11a-tetrahydro-3H,5H-spiro[benzo[e]pyrrolo[1,2-a][1,4]diazepin-2,1′-cyclopropane]-5-one (118 mg, 0.31 mmol) were dissolved in DMF (2 mL), followed by an addition of potassium carbonate (49 mg, 0.47 mmol), and reacted under stirring for 16 hours. The reaction was quenched with water, and the reaction mixture was extracted with ethyl acetate 3 times (20 mL×3). The organic phases were combined, washed with saturated brine, dried and concentrated, and purified by silica gel column purification (MeOH/DCM=0% to 5%) to obtain the title compound (81 mg, 0.09 mmol).

Its structural characterization data are as follows:

ESI-MS (m/z): 806.3 [M+H]⁺

### Step 11: Preparation of (S)-2-(4-aminophenyl)-8-((5-(((S)-7-cyclopropyloxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-7-methoxy-1,11a-dihydro-5H-benzo[e]pyrrolo[1,2-a][1,4]diazepin-5,11(10H)-dione (7-11)

Tert-butyl (4-((S)-8-((5-(((S)-7-cyclopropyloxy-5-oxo-5,10,11,11a-tetrahydro-1H,3H-spiro[benzo[e]]pyrrolo[1,2-a][1,4]diazepin-2,1′-cyclopropane]-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (81 mg, 0.09 mmol) was dissolved in dichloromethane (1 mL), followed by a dropwise addition of trifluoroacetic acid (0.6 mL) under stirring. After the addition was completed, the reaction mixture was stirred continuously for 1 hour, then concentrated under reduced pressure, purified by reverse phase column chromatography (acetonitrile/1% formic acid aqueous solution=0% to 70%) and then freeze-dried to obtain the title compound (3.5 mg, 0.005 mmol).

Its structural characterization data are as follows: ESI-MS (m/z): 706.3 [M+H]⁺

¹H NMR (400 MHz, DMSO): δ 7.58 (s, 1H), 7.27 (d, J=11.8 Hz, 2H), 7.11 (d, J=8.4 Hz, 2H), 6.57-6.51 (m, 2H), 6.29 (d, J=9.0 Hz, 2H), 5.19 (s, 2H), 4.18-4.07 (m, 1H), 4.01-3.88 (m, 4H), 3.86-3.80 (m, 1H), 3.67 (s, 3H), 3.57-3.51 (m, 4H), 3.27-3.10 (m, 4H), 2.73-2.65 (m, 1H), 2.02-1.93 (m, 1H), 1.88-1.71 (m, 5H), 1.61-1.48 (m, 2H), 1.24 (s, 2H), 0.88-0.75 (m, 1H), 0.69-0.48 (m, 6H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 22.00 | 90 | 10 | 28 |

### Preparation Example 6: Preparation of (S)-8-((5-(((S)-2-(4-aminophenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one-11,11-d2 (7-21)

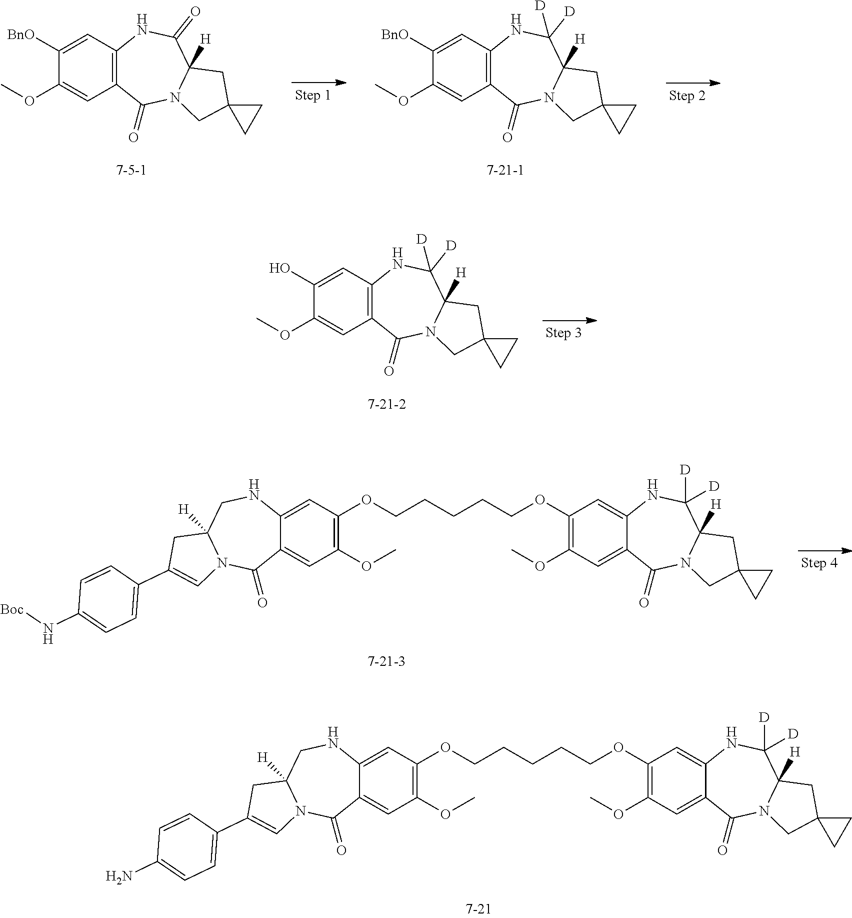 [SMILES: COc1cc2c(cc1OCc1ccccc1)NC(=O)[C@@H]1CC3(CC3)CN1C2=O ; [2H]C1([2H])Nc2cc(OCc3ccccc3)c(OC)cc2C(=O)N2CC3(CC3)C[C@H]21 ; [2H]C1([2H])Nc2cc(O)c(OC)cc2C(=O)N2CC3(CC3)C[C@H]21 ; [2H]C1([2H])Nc2cc(OCCCCCOc3cc4c(cc3OC)C(=O)N3C=C(c5ccc(NC(=O)OC(C)(C)C)cc5)C[C@H]3CN4)c(OC)cc2C(=O)N2CC3(CC3)C[C@H]21 ; [2H]C1([2H])Nc2cc(OCCCCCOc3cc4c(cc3OC)C(=O)N3C=C(c5ccc(N)cc5)C[C@H]3CN4)c(OC)cc2C(=O)N2CC3(CC3)C[C@H]21]

### Step 1: Preparation of (S)-8-(benzyloxy)-7-methoxy-1,10,11,11a-tetrahydro-3H,5H-spiro[benzo[e]pyrrolo[1,2-a][1,4]diazepin-2,1′-cyclopropane]-5-one-11,11-d2 (7-21-1)

(S)-8-(Benzyloxy)-7-methoxy-1,11a-dihydro-3H,5H-spiro[benzo[e]pyrrolo[1,2-a][1,4]diazepin-2,1′-cyclopropane]-5,11(10H)-dione (1.00 g, 2.64 mmol) was dissolved in tetrahydrofuran (3.00 mL), and cooled to 0° C. A tetrahydrofuran solution of deuterated borane (0.75 M, 7.05 mL) was added dropwise to the reaction system under nitrogen protection. The reaction system was warmed to 25° C. and continuously stirred for 2 hours. Then methanol (100 mL) was added to the reaction system, which was stirred for half an hour and directly concentrated to obtain a crude product of the title compound (1.00 g, crude), which was directly used in the next step without purification.

Its structural characterization data are as follows:

ESI-MS (m/z): 367.2 [M+H]⁺

### Step 2: Preparation of (S)-8-hydroxy-7-methoxy-1,10,11,11a-tetrahydro-3H,5H-spiro [benzo[e]pyrrolo[1,2-a][1,4]diazepin-2,1′-cyclopropane]-5-one-11,11-d2(7-21-2)

(S)-8-(Benzyloxy)-7-methoxy-1,10,11,11a-tetrahydro-3H,5H-spiro[benzo[e]pyrrolo[1,2-a][1,4]diazepin-2,1′-cyclopropane]-5-one-11,11-d2 (1.00 g, 2.73 mmol) was dissolved in methanol (10.0 mL), subjected to nitrogen replacement for 3 times, followed by an addition of Pd/C (0.20 g, 187 μmol, 10% purity), introduced with hydrogen gas, and reacted continuously for 2 hours. The reaction mixture was filtered through a silica gel pad, and directly concentrated to obtain a crude product, which was purified by silica gel column (dichloromethane/methanol=100/1 to 97/3) and concentrated again to obtain the title compound (500 mg, 1.77 mmol).

Its structural characterization data are as follows:

ESI-MS (m/z): 277.1 [M+H]⁺

¹H NMR: (400 MHz, CD3OD) δ 7.36 (s, 1H), 6.52 (s, 1H), 3.95-4.04 (m, 1H), 3.86 (s, 3H), 3.63 (d, J=11.6 Hz, 1H), 3.49 (d, J=12.0 Hz, 1H), 2.26 (dd, J=12.8, 8.0 Hz, 1H), 1.68 (dd, J=12.8, 4.4 Hz, 1H), 0.69-0.79 (m, 2H), 0.62-0.69 (m, 2H)

### Step 3: Preparation of tert-butyl (4-((S)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl-11,11-d2)oxy)pentyl)oxy)-5-oxo-5,10,11,11a-tetrahydro-1H-benzo[e]pyrrolo[1,2-a][1,4]diazepin-2-yl)phenyl)carbamate (7-21-3)

Tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (21.23 mg, 36.19 μmol) and (S)-8-hydroxy-7-methoxy-1,10,11,11a-tetrahydro-3H,5H-spiro[benzo[e]pyrrolo[1,2-a][1,4]diazepin-2,1′-cyclopropane]-5-one-11,11-d2 (10 mg, 36.19 μmol) were dissolved in DMF (1 mL), followed by an addition of potassium carbonate (14.98 mg, 108.57 μmol), and reacted under stirring for 16 hours. The reaction was quenched with water, and the reaction mixture was extracted with ethyl acetate 3 times (10 mL×3). The organic phases were combined, washed with saturated brine, dried, concentrated, and purified by silica gel column (MeOH/DCM=0% to 5%) to obtain the title compound (20 mg, 25.58 μmol).

Its structural characterization data are as follows:

ESI-MS (m/z): 794.4 [M+H]⁺

### Step 4: Preparation of (S)-8-((5-(((S)-2-(4-aminophenyl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one-11,11-d2 (7-21)

Tert-butyl (4-((S)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl-11,11-d2)oxy)pentyl)oxy)-5-oxo-5,10,11,11a-tetrahydro-1H-benzo[e]pyrrolo[1,2-a][1,4]diazepin-2-yl)phenyl)carbamate (20 mg, 25.58 μmol) was dissolved in dichloromethane (1 mL), followed by a dropwise addition of trifluoroacetic acid (0.5 mL) under stirring. After the addition was complete, the reaction mixture was stirred continuously for 1 hour, concentrated under reduced pressure, then purified by reverse phase column chromatography (acetonitrile/l % formic acid aqueous solution=0% to 70%) and freeze-dried to obtain the title compound (4.7 mg, 6.82 μmol).

Its structural characterization data are as follows:

ESI-MS (m/z): 682.4 [M+H]⁺

¹H NMR (400 MHz, DMSO) δ 7.28 (d, J=13.4 Hz, 2H), 7.27 (s, 1H), 7.15 (d, J=8.5 Hz, 2H), 6.59 (d, J=8.6 Hz, 2H), 6.30 (d, J=10.2 Hz, 2H), 3.95 (dd, J=12.0, 6.1 Hz, 4H), 3.66 (d, J=7.5 Hz, 6H), 3.59-3.51 (m, 2H), 3.37-3.15 (m, 4H), 2.76-2.67 (m, 2H), 2.03-1.92 (m, 2H), 1.85-1.75 (m, 4H), 1.62-1.52 (m, 2H), 0.68-0.58 (m, 2H), 0.56-0.48 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 7: Preparation of (S)-2-(4-aminophenyl)-7-methoxy-8-((5-(((S)-7-methoxy-5-oxo-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-22)

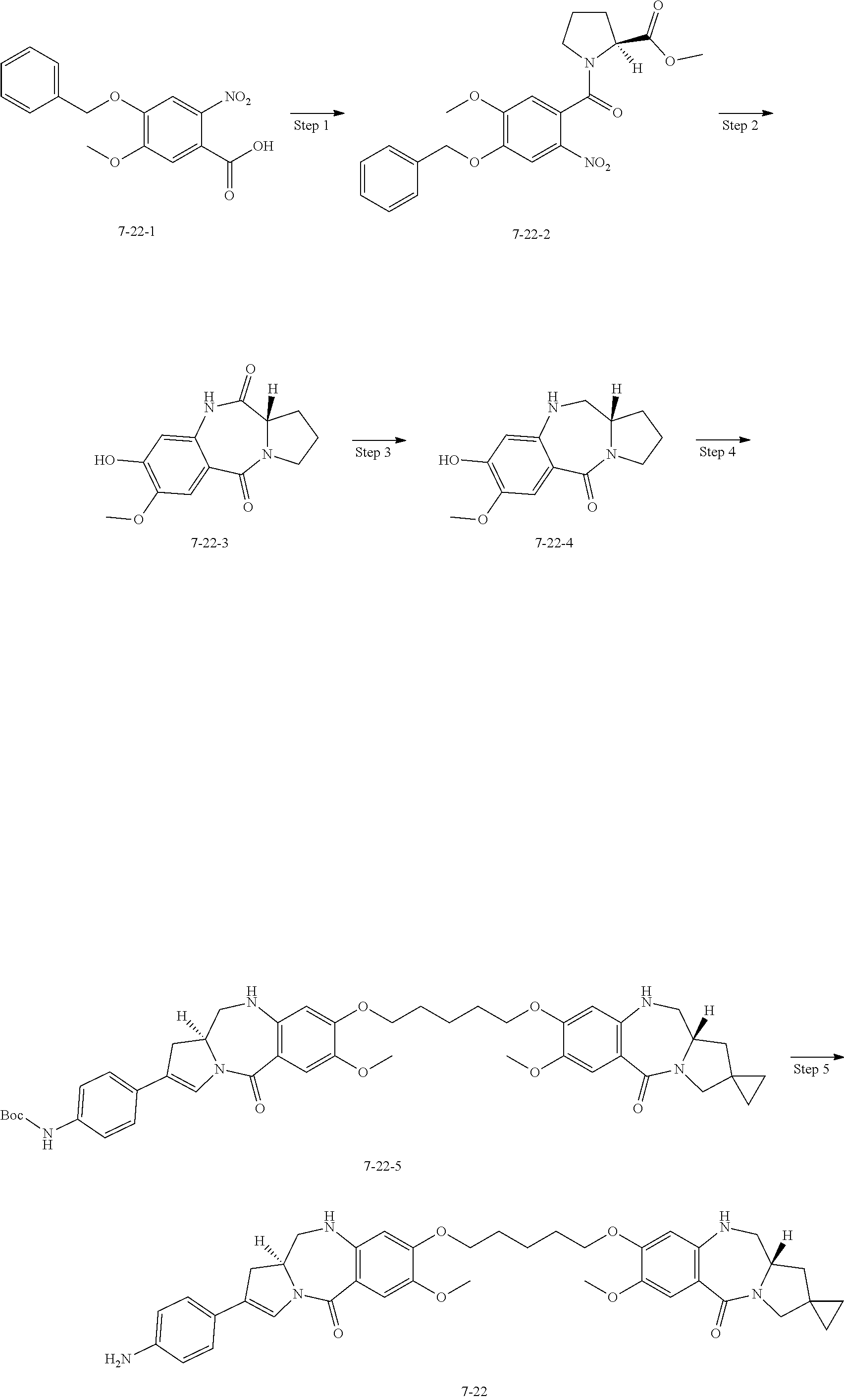 [SMILES: COc1cc(C(=O)O)c([N+](=O)[O-])cc1OCc1ccccc1 ; COC(=O)[C@@H]1CCCN1C(=O)c1cc(OC)c(OCc2ccccc2)cc1[N+](=O)[O-] ; COc1cc2c(cc1O)NC(=O)[C@@H]1CCCN1C2=O ; COc1cc2c(cc1O)NC[C@@H]1CCCN1C2=O ; COc1cc2c(cc1OCCCCCOc1cc3c(cc1OC)C(=O)N1CC4(CC4)C[C@H]1CN3)NC[C@@H]1CC(c3ccc(NC(=O)OC(C)(C)C)cc3)=CN1C2=O ; COc1cc2c(cc1OCCCCCOc1cc3c(cc1OC)C(=O)N1CC4(CC4)C[C@H]1CN3)NC[C@@H]1CC(c3ccc(N)cc3)=CN1C2=O]

### Step 1: Preparation of methyl (4-(benzyloxy)-5-methoxy-2-nitrobenzoyl)-L-proline (7-22-2)

4-(Benzyloxy)-5-methoxy-2-nitrobenzoic acid (3.00 g, 9.89 mmol), and L-proline methyl ester hydrochloride (1.97 g, 11.87 mmol) were dissolved in DMF (30 mL), followed by an addition of HATU (5.64 g, 14.84 mmol) and DIPEA (3.84 g, 29.68 mmol) in sequence, and reacted at 25° C. for 1 hour. The reaction mixture was poured into water (100 ml), and the product was extracted with EA/PE=1/1 (200 ml), washed with brine (30 ml), and concentrated to obtain the title compound (4.10 g, 9.89 mmol), which was used directly in the next step without purification.

Its structural characterization data are as follows:

MS m/z (ESI): 415.1 [M+H]⁺

### Step 2: Preparation of (S)-8-hydroxy-7-methoxy-1,2,3,11a-tetrahydro-5H-benzo[e]pyrrolo [1,2-a][1,4]diazepin-5,11(10H)-dione (7-22-3)

The compound (4-(benzyloxy)-5-methoxy-2-nitrobenzoyl)-L-proline methyl ester (3.80 g, 8.98 mmol) was dissolved in methanol (40 mL) and tetrahydrofuran (40 mL), followed by an addition of 10% palladium-on-carbon (380 mg) and HOAc (1.08 g, 17.95 mmol). The air was removed under vacuum, hydrogen was introduced, and the reaction was carried out at 25° C. for 16 hours. The palladium-on-carbon was filtered off, and the filtrate was concentrated to obtain the title compound (2.35 g, 8.98 mmol), which was used directly in the next step without purification.

Its structural characterization data are as follows:

MS m/z (ESI): 263.1 [M+H]⁺

### Step 3: Preparation of (S)-8-hydroxy-7-methoxy-1,2,3,10,11,11a-hexahydro-5H-benzo[e]pyrrolo[1,2-a][1,4]diazepin-5-one (7-22-4)

The compound (S)-8-hydroxy-7-methoxy-1,2,3,11a-tetrahydro-5H-benzo[e]pyrrolo[1,2-a][1,4]diazepin-5,11(10H)-dione (100.0 mg, 381.30 μmol) was dissolved in THF (3 mL), followed by an addition of borane tetrahydrofuran solution (4 M, 476.62 μL), and reacted under stirring at 25° C. for 1 hour. The reaction mixture was poured into methanol (3 ml), adjusted to pH=5-6 with 20% formic acid aqueous solution, and concentrated to obtain a crude product, which was purified by C18 reverse column (H₂O:ACN=0 to 30%, 0.05% FA) and freeze-dried to obtain the title compound (95.0 mg, 306.11 μmol).

Its structural characterization data are as follows:

MS m/z (ESI): 249.1 [M+H]⁺

### Step 4: Preparation of tert-butyl (4-((S)-7-methoxy-8-((5-(((S)-7-methoxy-5-oxo-2,3,5,10,11,11a-hexahydro-1H-benzo[e]pyrrolo[1,2-a][1,4]diazepin-8-yl)oxy)pentyl)oxy)-5-oxo-5,10,11,11a-tetrahydro-1H-benzo[e]pyrrolo[1,2-a][1,4]diazepin-2-yl)phenyl)carbamate (7-22-5)

Allyl (S)-8-((5-bromopentyl)oxy)-2-(4-((tert-butyloxycarbonyl)amino)phenyl)-7-methoxy-5-oxo-11,11a-dihydro-1H-benzo[e]pyrrolo[1,2-a][1,4]diazepin-10(5H)-carboxylate (20 mg, 34.10 μmol) and the compound (S)-8-hydroxy-7-methoxy-1,2,3,10,11,11a-hexahydro-5H-benzo[e]pyrrolo[1,2-a][1,4]diazepin-5-one (12.70 mg, 51.15 μmol, FR) were dissolved in DMF (1 mL), followed by an addition of Cs₂CO₃ (33.33 mg, 102.30 μmol), heated to 40° C. and reacted for 16 hours. EA (20 ml) and H₂O (6 ml) were added to the reaction system, which was stirred for 5 minutes. The EA phase was separated, washed with brine, and concentrated to obtain a crude product, which was purified by thin layer chromatography (MeOH/DCM=10%) to obtain the title compound (15.0 mg, 19.90 μmol).

Its structural characterization data are as follows:

MS m/z (ESI): 754.4 [M+H]⁺

### Step 5: Preparation of (S)-2-(4-aminophenyl)-7-methoxy-8-((5-(((S)-7-methoxy-5-oxo-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-22)

The compound tert-butyl (4-((S)-7-methoxy-8-((5-(((S)-7-methoxy-5-oxo-2,3,5,10,11,11a-hexahydro-1H-benzo[e]pyrrolo[1,2-a][1,4]diazepin-8-yl)oxy)pentyl)oxy)-5-oxo-5,10,11,11a-tetrahydro-1H-benzo[e]pyrrolo[1,2-a][1,4]diazepin-2-yl)phenyl)carbamate (15.0 mg, 19.90 μmol) was dissolved in DCM (0.5 mL), followed by an addition of trifluoroacetic acid (226.87 mg, 1.99 mmol), and reacted at 25° C. for 1 hour. The reaction mixture was concentrated to obtain a crude product, which was purified by preparative HPLC and freeze-dried to obtain the title compound (7.10 mg, 10.53 μmol).

Its preparation method was as follows:
Chromatographic column: Waters SunFire Prep C18 OBD (5 μm*19 mm*150 mm)
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 20 | 80 | 28 |
| 4.00 | 20 | 80 | 28 |
| 24.00 | 90 | 10 | 28 |

Its structural characterization data are as follows:

MS m/z (ESI): 654.4 [M+H]⁺

¹H NMR (400 MHz, MeOD): δ 7.38 (dd, J=8.2, 2.3 Hz, 3H), 6.91 (d, J=8.9 Hz, 2H), 6.27 (d, J=9.3 Hz, 2H), 4.24 (s, 1H), 4.00 (dd, J=9.9, 6.3 Hz, 4H), 3.80 (s, 3H), 3.77 (s, 3H), 3.76 (s, 3H), 3.64 (s, 1H), 3.61 (s, 1H), 3.56 (d, J=11.1 Hz, 2H), 3.50-3.46 (m, 2H), 3.41 (s, 1H), 3.40-3.33 (m, 3H), 2.80 (d, J=13.5 Hz, 1H), 2.08 (d, J=5.0 Hz, 1H), 1.95-1.86 (m, 5H), 1.69 (d, J=6.7 Hz, 2H), 0.71-0.68 (m, 1H), 0.61 (dd, J=12.4, 6.0 Hz, 3H).

### Preparation Example 8: Preparation of (S)-2-(4-aminophenyl)-8-((5-(((2R,11aS)-2-hydroxy-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-23)

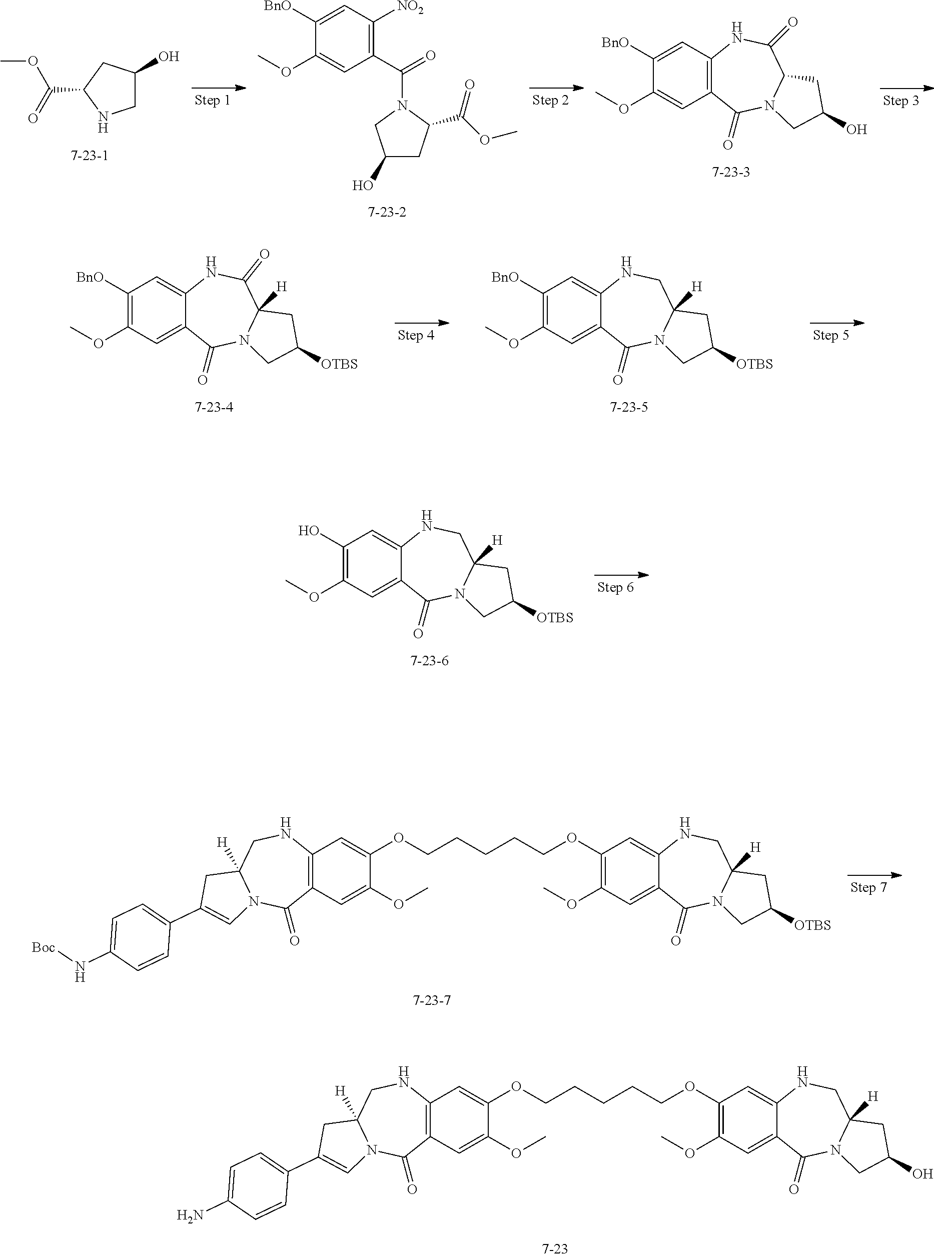

### Step 1: Preparation of methyl (2S,4R)-1-(4-(benzyloxy)-5-methoxy-2-nitrobenzoyl)-4-hydroxypyrrolidine-2-carboxylate (7-23-2)

4-Benzyloxy-5-methoxy-2-nitro-benzoic acid (10 g, 32.97 mmol), methyl (2S,4R)-4-hydroxypyrrolidine-2-carboxylate hydrochloride (5.27 g, 36.27 mmol) and HATU (18.81 g, 49.46 mmol) were dissolved in DMF (50 mL), followed by a dropwise addition of DIPEA (12.78 g, 98.92 mmol), and reacted under stirring for 2 hours. The reaction mixture was treated with water and EA, stirred, and allowed to stand for liquid separation. The aqueous phase was extracted with EA twice. The organic phases were combined, washed with saturated brine twice, dried, concentrated, and purified by silica gel column (eluent: 30-65% ethyl acetate/petroleum ether) and concentrated again to obtain the title compound (14 g, 32.53 mmol).

### Step 2: Preparation of (2R,11aS)-8-(benzyloxy)-2-hydroxy-7-methoxy-1,2,3,11a-tetrahydro-5H-benzo[e]pyrrolo[1,2-a][1,4]diazepin-5,11(10H)-dione (7-23-3)

Methyl (2S,4R)-1-(4-(benzyloxy)-5-methoxy-2-nitrobenzoyl)-4-hydroxypyrrolidine-2-carboxylate (14 g, 32.53 mmol) was dissolved in methanol (700 mL), followed by an addition of saturated aqueous ammonium chloride solution (70 mL) and zinc powder (21.27 g, 325.27 mmol) in batches, heated to 80° C. and reacted under refluxing for 16 hours. The reaction mixture was cooled to room temperature and filtered. The filtrate was concentrated under reduced pressure and purified by silica gel column (eluent: ethyl acetate) to obtain the title compound (5.3 g, 14.39 mmol).

Its structural characterization data are as follows:

MS m/z (ESI): 368.5[M+H]⁺

### Step 3: Preparation of (2R,11aS)-8-(benzyloxy)-2-((tert-butyldimethylsilyl)oxy)-7-methoxy-1,2,3,11a-tetrahydro-5H-benzo[e]pyrrolo[1,2-a][1,4]diazepin-5,11(10H)-dione (7-23-4)

(2R,11aS)-8-(Benzyloxy)-2-hydroxy-7-methoxy-1,2,3,11a-tetrahydro-5H-benzo[e]pyrrolo[1,2-a][1,4]diazepin-5,11(10H)-dione (1 g, 2.71 mmol) was dissolved in DMF (25 mL), followed by an addition of imidazole (1.85 g, 27.15 mmol) and TBSCl (1.12 g, 13.57 mmol), and reacted for 16 hours. Water and ethyl acetate were added to the reaction solution, stirred and allowed to stand for liquid separation. The organic phase was washed with saturated brine three times, concentrated under reduced pressure, then purified by silica gel column (eluent: 50% ethyl acetate/petroleum ether) and concentrated again to obtain the title compound (1.02 g, 2.11 mmol).

### Step 4: Preparation of (2R,11aS)-8-(benzyloxy)-2-((tert-butyldimethylsilyl)oxy)-7-methoxy-1,2,3,10,11,11a-hexahydro-5H-benzo[e]pyrrolo[1,2-a][1,4]diazepin-5-one (7-23-5)

(2R,11aS)-8-(Benzyloxy)-2-((tert-butyldimethylsilyl)oxy)-7-methoxy-1,2,3,11a-tetrahydro-5H-benzo[e]pyrrolo[1,2-a][1,4]diazepin-5,11(10H)-dione (500 mg, 1.04 mmol) was dissolved in tetrahydrofuran (5 mL) and cooled to 0° C. Then tetrahydrofuran borane complex (10.3 mL) was added dropwise. After the addition was completed, the reaction was heated to room temperature and reacted for 3 hours. Methanol (10 ml) was added dropwise to the reaction mixture, then the mixture was concentrated to obtain a crude product of the title compound, which was purified by silica gel column (ethyl acetate/petroleum ether=0% to 70%) and concentrated to obtain the title compound (120 mg, 0.26 mmol).

Its structural characterization data are as follows:

ESI-MS (m/z): 453.3 [M+H]⁺

### Step 5: Preparation of (2R,11aS)-2-((tert-butyldimethylsilyl)oxy)-8-hydroxy-7-methoxy-1,2,3,10,11,11a-hexahydro-5H-benzo[e]pyrrolo[1,2-a][1,4]diazepin-5-one (7-23-6)

(2R,11aS)-8-(Benzyloxy)-2-((tert-butyldimethylsilyl)oxy)-7-methoxy-1,2,3,10,11,11a-hexahydro-5H-benzo[e]pyrrolo[1,2-a][1,4]diazepin-5-one (120 mg, 0.26 mmol) was dissolved in tetrahydrofuran (3 mL), followed by an addition of 20% palladium on carbon (12 mg), subjected to hydrogen replacement three times, heated to 45° C. and reacted under stirring for 3 hours. After filtration through diatomaceous earth, the filtrate was concentrated to obtain a crude product of the title compound (90 mg, 0.23 mmol), which was used directly in the next step without purification.

Its structural characterization data are as follows:

ESI-MS (m/z): 379.2 [M+H]⁺

### Step 6: Preparation of tert-butyl (4-((S)-8-((5-(((2R,11aS)-2-((tert-butyldimethylsilyl)oxy)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-5-oxo-5,10,11,11-tetrahydro-1H-benzo[e]pyrrolo[1,2-a][1,4]diazepin-2-yl)phenyl)carbamate (7-23-7)

Tert-butyl (S)-(4-(8-((5-bromopentyl)oxy)-7-methoxy-5,11-dioxy-50,11,11-tetrahydro-1H-benzo[e]pyrrolo[1,2-a][1,4]diazepin-2-yl)phenyl)carbamate (135 mg, 0.23 mmol) and (2R,11aS)-2-((tert-butyldimethylsilyl)oxy)-8-hydroxy-7-methoxy-1,2,3,10,11,11a-hexahydro-5H-benzo[e]pyrrolo[1,2-a][1,4]diazepin-5-one (90 mg, 0.23 mmol) were dissolved in DMF (2 mL), followed by an addition of potassium carbonate (64 mg, 0.47 mmol), and then reacted under stirring for 16 hours. The reaction was quenched with water, and the reaction mixture was extracted with ethyl acetate 3 times (20 mL×3). The organic phases were combined, washed with saturated brine, dried, concentrated, purified by silica gel column (MeOH/DCM=0% to 5%) and concentrated again to obtain the title compound (82 mg, 0.092 mmol).

Its structural characterization data are as follows:

ESI-MS (m/z): 884.3 [M+H]⁺

### Step 7: Preparation of (S)-2-(4-aminophenyl)-8-((5-(((2R,11aS)-2-hydroxy-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-23)

Tert-butyl (4-((S)-8-((5-(((2R,11aS)-2-((tert-butyldimethylsilyl)oxy)-7-methoxy-5-oxo-2,3, 5,10,11,11a-hexahydro-1H-benzo[e]pyrrolo[1,2-a][1,4]diazepin-8-yl)oxy)pentyl)oxy)-7-methoxy-5-oxo-5,10,11,11-tetrahydro-1H-benzo[e]pyrrolo[1,2-a][1,4]diazepin-2-yl)phenyl)carbamate (82 mg, 0.092 mmol) was dissolved in dichloromethane (5 mL), followed by a dropwise addition of trifluoroacetic acid (3 mL) under stirring. After the addition was completed, the reaction mixture was stirred continuously for 5 hours, concentrated under reduced pressure, then purified by reverse phase column chromatography (acetonitrile/1% formic acid aqueous solution=0% to 70%) and freeze-dried to obtain the title compound (8.4 mg, 0.012 mmol).

Its structural characterization data are as follows: ESI-MS (m/z): 670.3 [M+H]⁺

¹H NMR (400 MHz, DMSO): δ 7.37 (s, 1H), 7.29 (s, 1H), 7.26 (s, 1H), 7.11 (d, J=8.4 Hz, 2H), 6.53 (d, J=8.4 Hz, 2H), 6.31 (s, 1H), 6.26 (s, 1H), 5.28-5.10 (m, 2H), 5.08-4.79 (m, 1H), 4.21-4.11 (m, 2H), 3.97-3.88 (m, 4H), 3.89-3.76 (m, 1H), 3.66 (s, 3H), 3.64 (s, 3H), 3.55-3.47 (m, 4H), 3.25-3.20 (m, 2H), 3.01-2.93 (m, 1H), 2.68-2.65 (m, 1H), 2.12-2.04 (m, 1H), 1.85-1.76 (m, 4H), 1.74-1.66 (m, 1H), 1.61-1.52 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 80 | 28 |
| 2.00 | 30 | 80 | 28 |
| 24.00 | 90 | 10 | 28 |

### Preparation Example 9: Preparation of (S)-2-(4-aminophenyl)-8-((5-(((2S,11aS)-2-hydroxy-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-24)

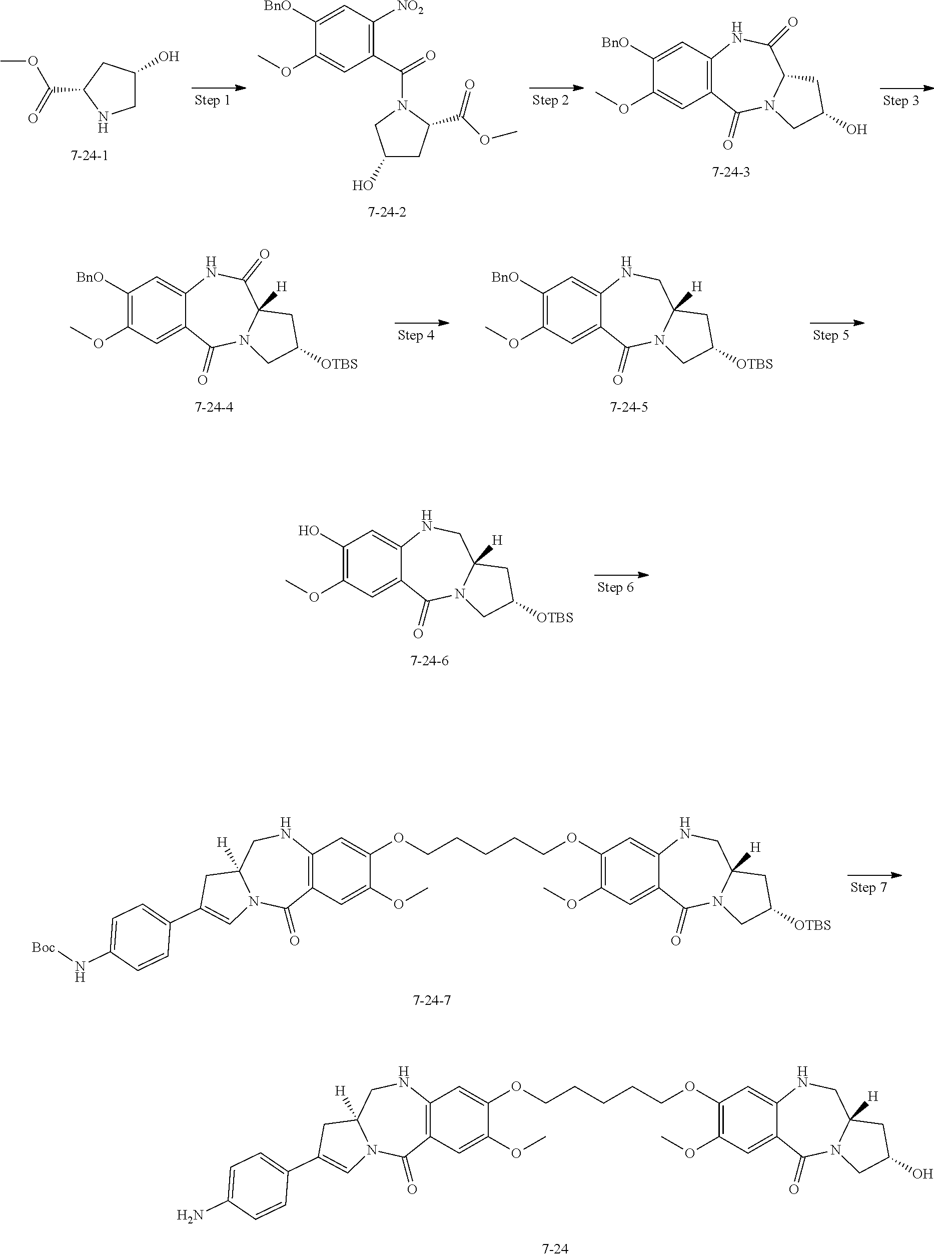

### Step 1: Preparation of methyl (2S,4S)-1-(4-(benzyloxy)-5-methoxy-2-nitrobenzoyl)-4-hydroxypyrrolidine-2-carboxylate (7-24-2)

4-(Benzyloxy)-5-methoxy-2-nitrobenzoic acid (1.0 g, 3.3 mmol) and methyl (2S,4S)-4-hydroxypyrrolidine-2-carboxylate (503 mg, 3.46 mmol) were dissolved in DMF (10 mL), followed by an addition of HATU (2.51 g, 6.51 mmol) and DIPEA (1.28 g, 9.9 mmol), and reacted at room temperature for 2 hours. 50 mL of water was added to the reaction solution, and then 70 mL of ethyl acetate was added for extraction. After liquid separation, the organic phase was dried over anhydrous sodium sulfate, filtered and concentrated to obtain a crude product, which was purified by silica gel column (ethyl acetate/petroleum ether=0% to 50%) and concentrated to obtain the title compound (1.36 g, 3.16 mmol).

Its structural characterization data are as follows:

ESI-MS (m/z): 431.1 [M+H]⁺

### Step 2: Preparation of (2S,11aS)-8-(benzyloxy)-2-hydroxy-7-methoxy-1,2,3,11a-tetrahydro-5H-benzo[e]pyrrolo[1,2-a][1,4]diazepin-5,11(10H)-dione (7-24-3)

Methyl (2S,4S)-1-(4-(benzyloxy)-5-methoxy-2-nitrobenzoyl)-4-hydroxypyrrolidine-2-carboxylate (1.36 g, 3.16 mmol) was dissolved in methanol (52 mL), followed by an addition of 10% aqueous ammonium chloride solution (10 mL) and zinc powder (2.43 g, 37.2 mmol), heated to 40° C. and reacted for 1 hour, then heated to 80° C. and reacted overnight, and concentrated to obtain a crude product, to which 50 mL of ethyl acetate was added for dissolution, and washed 3 times with saturated brine. The organic phase was dried over anhydrous sodium sulfate, filtered and concentrated to obtain a crude product, which was purified by reverse phase column chromatography (acetonitrile/1% formic acid aqueous solution=0% to 70%) and freeze-dried to obtain the title compound (1.3 g, 3.53 mmol).

Its structural characterization data are as follows:

ESI-MS (m/z): 369.2[M+H]⁺

### Step 3: Preparation of (2S,11aS)-8-(benzyloxy)-2-((tert-butyldimethylsilyl)oxy)-7-methoxy-1,2,3,11a-tetrahydro-5H-benzo[e]pyrrolo[1,2-a][1,4]diazepin-5,11(10H)-dione (7-24-4)

(2S,11aS)-8-(Benzyloxy)-2-hydroxy-7-methoxy-1,2,3,11a-tetrahydro-5H-benzo[e]pyrrolo[1,2-a][1,4]diazepin-5,11(10H)-dione (1.3 g, 3.53 mmol) was dissolved in DMF (15 mL), followed by an addition of tert-butyldimethylsilyl chloride (2.66 g, 17.64 mmol) and imidazole (2.4 g, 35.3 mmol), and reacted at room temperature for 16 hours. 60 mL of water was added to the reaction mixture, followed by extraction with 80 mL of ethyl acetate. After liquid separation, the organic phase was dried over anhydrous sodium sulfate, filtered and concentrated to obtain a crude product, which was purified by silica gel column (ethyl acetate/petroleum ether=0% to 50%) and concentrated to obtain the title compound (1.38 g, 2.86 mmol).

Its structural characterization data are as follows:

ESI-MS (m/z): 483.2[M+H]⁺

### Step 4: Preparation of (2S,11aS)-8-(benzyloxy)-2-((tert-butyldimethylsilyl)oxy)-7-methoxy-1,2,3,10,11,11a-hexahydro-5H-benzo[e]pyrrolo[1,2-a][1,4]diazepin-5-one (7-24-5)

(2S,11aS)-8-(Benzyloxy)-2-((tert-butyldimethylsilyl)oxy)-7-methoxy-1,2,3,11a-tetrahydro-5H-benzo[e]pyrrolo[1,2-a][1,4]diazepin-5,11(10H)-dione (500 mg, 1.04 mmol) was dissolved in tetrahydrofuran (5 mL) and cooled to 0° C. Then tetrahydrofuran borane complex (10.3 mL) was added dropwise. After the addition was completed, the reaction mixture was heated to room temperature and reacted for 3 hours. Methanol (10 ml) was added to the reaction mixture, and then the mixture was concentrated to obtain a crude product of the title compound, which was purified by silica gel column (ethyl acetate/petroleum ether=0% to 70%) and concentrated to obtain the title compound (60 mg, 0.13 mmol).

Its structural characterization data are as follows:

ESI-MS (m/z): 469.2 [M+H]⁺

### Step 5: Preparation of (2S,11aS)-2-((tert-butyldimethylsilyl)oxy)-8-hydroxy-7-methoxy-1,2,3,10,11,11a-hexahydro-5H-benzo[e]pyrrolo[1,2-a][1,4]diazepin-5-one (7-24-6)

(2S,11aS)-8-(Benzyloxy)-2-((tert-butyldimethylsilyl)oxy)-7-methoxy-1,2,3,10,11,11a-hexahydro-5H-benzo[e]pyrrolo[1,2-a][1,4]diazepin-5-one (60 mg, 0.13 mmol) was dissolved in tetrahydrofuran (3 mL), followed by an addition of 20% palladium-on-carbon (6 mL), subjected to hydrogen replacement three times, then kept warm to 25° C. and stirred for 2 hours. After filtration through diatomaceous earth, the filtrate was concentrated to obtain a crude product of the title compound (46 mg, 0.12 mmol), which was used directly in the next step without purification.

Its structural characterization data are as follows:

ESI-MS (m/z): 379.2 [M+H]⁺

### Step 6: Preparation of tert-butyl (4-((S)-8-((5-(((2S,11aS)-2-((tert-butyldimethylsilyl)oxy)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (7-24-6)

Tert-butyl (S)-(4-(8-((5-bromopentyl)oxy)-7-methoxy-5,11-dioxy-5,10,11,11-tetrahydro-1H-benzo[e]pyrrolo[1,2-a][1,4]diazepin-2-yl)phenyl)carbamate (71 mg, 0.12 mmol) and (2S,11aS)-2-((tert-butyldimethylsilyl)oxy)-8-hydroxy-7-methoxy-1,2,3,10,11,11a-hexahydro-5H-benzo[e]pyrrolo[1,2-a][1,4]diazepin-5-one (46 mg, 0.12 mmol) were dissolved in DMF (2 mL), followed by an addition of potassium carbonate (33 mg, 0.24 mmol), and reacted under stirring for 16 hours. The reaction was quenched with water, and the reaction mixture was extracted with ethyl acetate three times (20 mL×3). The organic phases were combined, washed with saturated brine, dried, concentrated, and purified by silica gel column (MeOH/DCM=0% to 5%) to obtain the title compound (39 mg, 0.04 mmol).

Its structural characterization data are as follows:

ESI-MS (m/z): 884.3 [M+H]⁺

### Step 7: Preparation of (S)-2-(4-aminophenyl)-8-((5-(((2S,11aS)-2-hydroxy-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-24)

Tert-butyl (4-((S)-8-((5-(((2S,11aS)-2-((tert-butyldimethylsilyl)oxy)-7-methoxy-5-oxo-2,3,5, 10,11,11a-hexahydro-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (39 mg, 0.04 mmol) was dissolved in dichloromethane (3 mL), followed by a dropwise addition of trifluoroacetic acid (2 mL) under stirring. After the addition was completed, the reaction mixture was stirred continuously for 5 hours, concentrated under reduced pressure, purified by reverse phase column chromatography (acetonitrile/l % formic acid aqueous solution=0% to 70%) and then freeze-dried to obtain the title compound (7 mg, 0.01 mmol).

Its structural characterization data are as follows: ESI-MS (m/z): 670.3 [M+H]⁺

¹H NMR (400 MHz, DMSO): δ 7.28 (d, J=4.0 Hz, 2H), 7.26 (s, 1H), 7.12 (d, J=8.4 Hz, 2H), 6.54 (d, J=8.4 Hz, 2H), 6.30 (s, 1H), 6.26 (s, 1H), 5.37-5.14 (m, 1H), 5.10-5.03 (m, 1H), 4.24-4.06 (m, 2H), 3.96-3.84 (m, 2H), 3.66 (s, 3H), 3.63 (s, 3H), 3.61-3.56 (m, 1H), 3.55-3.48 (m, 1H), 3.47-3.40 (m, 3H), 3.26-3.21 (m, 3H), 2.68-2.64 (m, 1H), 2.34-2.30 (m, 1H), 1.85-1.76 (m, 4H), 1.71-1.63 (m, 1H), 1.62-1.51 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 80 | 28 |
| 2.00 | 30 | 80 | 28 |
| 24.00 | 90 | 10 | 28 |

### Preparation Example 10: Preparation of (S)-2-(4-aminophenyl)-7-methoxy-8-((5-(((2S,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-25)

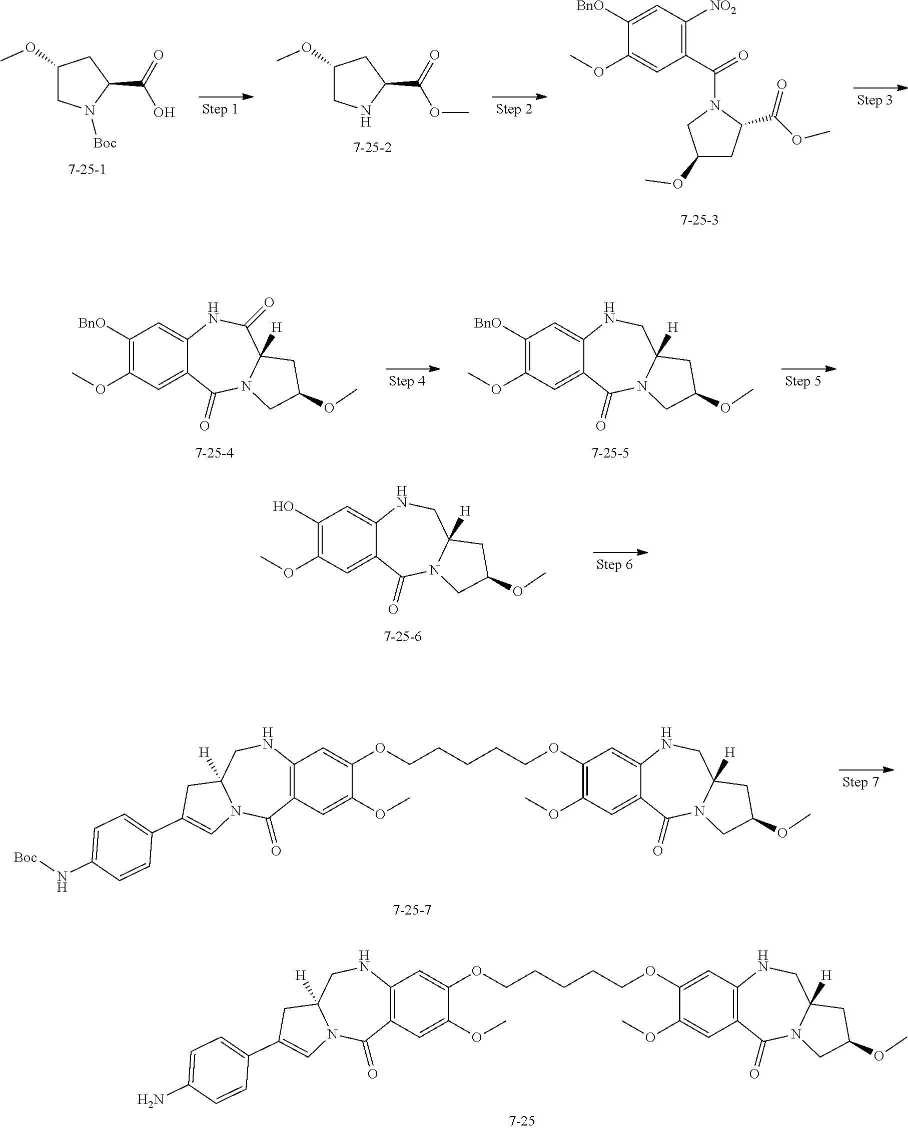

### Step 1: Preparation of methyl (2S,4R)-4-methoxypyrrolidine-2-carboxylate (7-25-2)

1-(Tert-butyl)2-methyl(2S,4R)-4-methoxypyrrolidine-1,2-dicarboxylic acid (1.0 g, 3.53 mmol, FR) was dissolved in methanol (10 mL), followed by an addition of HCl (4M in EA) (5 mL), and reacted at room temperature for 1 hour. The reaction mixture was concentrated to obtain a crude product (800.6 mg, 5.01 mmol), which was used directly in the next step without purification.

Its structural characterization data are as follows:

ESI-MS (m/z): 160.1 [M+H]⁺

### Step 2: Preparation of methyl (2R,4S)-1-(4-(benzyloxy)-5-methoxy-2-nitrobenzoyl)-4-methoxypyrrolidine-2-carboxylate (7-25-3)

Methyl (2S,4R)-4-methoxypyrrolidine-2-carboxylate (800.6 mg, 5.01 mmol) and 4-(benzyloxy)-5-methoxy-2-nitrobenzoic acid (1.18 g, 3.88 mmol, FR) were dissolved in DMF (10 mL), followed by an addition of HATU (1.48 g, 3.88 mmol) and DIPEA (1.36 g, 10.59 mmol), and reacted at room temperature for 1 hour. The reaction mixture was added to ethyl acetate (100 mL) and saturated brine (100 mL), stirred, and allowed to stand for liquid separation. The organic phase was dried over anhydrous sodium sulfate, filtered and concentrated to obtain a crude product, which was purified by silica gel column (ethyl acetate/petroleum ether=20% to 60%) and concentrated again to obtain the title compound (1.56 g, 3.51 mmol).

Its structural characterization data are as follows:

ESI-MS (m/z): 445.2 [M+H]⁺

### Step 3: Preparation of (2R,11aS)-8-(benzyloxy)-2,7-dimethoxy-1,2,3,11a-tetrahydro-5H-benzo[e]pyrrolo[1,2-a][1,4]diazepin-5,11(10H)-dione (7-25-4)

Methyl (2S,4R)-1-(4-(benzyloxy)-5-methoxy-2-nitrobenzoyl)-4-(trifluoromethyl)pyrrolidin-2-carboxylate (1.56 g, 3.1 mmol) was dissolved in methanol (30 mL), followed by an addition of zinc powder (2.4 g, 31.1 mmol) and saturated aqueous ammonium chloride solution (40 mL), heated to 85° C. and reacted under refluxing for 16 hours. The reaction mixture was added to ethyl acetate (150 mL) and saturated brine (100 mL), stirred, and allowed to stand for liquid separation. The organic phase was dried over anhydrous sodium sulfate and concentrated to obtain a crude product of the title compound (1.13 g, 2.95 mmol), which was used directly in the next step without purification.

Its structural characterization data are as follows:

ESI-MS (m/z): 383.2 [M+H]⁺

### Step 4: Preparation of (2R,11aS)-8-(benzyloxy)-2,7-dimethoxy-1,2,3,10,11,11a-hexahydro-5H-benzo[e]pyrrolo[1,2-a][1,4]diazepin-5-one (7-25-5)

(2R,11aS)-8-(Benzyloxy)-2,7-dimethoxy-1,2,3,11a-tetrahydro-5H-benzo[e]pyrrolo[1,2-a][1,4]diazepin-5,11(10H)-dione (0.5 g, 1.3 mmol, FR) was dissolved in tetrahydrofuran (5 mL), and cooled to 0° C. Then, tetrahydrofuran borane complex (10.3 mL) was added dropwise. After the addition was completed, the reaction mixture was heated to room temperature, and reacted for 3 hours. Methanol (10 ml) was added dropwise to the reaction mixture, then the mixture was concentrated to obtain a crude product of the title compound, which was purified by silica gel column (ethyl acetate/petroleum ether=0% to 70%) and concentrated to obtain the title compound (80 mg, 0.22 mmol).

Its structural characterization data are as follows:

ESI-MS (m/z): 369.1 [M+H]⁺

### Step 5: Preparation of (2R,11aS)-8-hydroxy-2,7-dimethoxy-1,2,3,10,11,11a-hexahydro-5H-benzo[e]pyrrolo[1,2-a][1,4]diazepin-5-one (7-25-6)

(2R,11aS)-8-(Benzyloxy)-2,7-dimethoxy-1,2,3,10,11,11a-hexahydro-5H-benzo[e]pyrrolo[1,2-a][1,4]diazepin-5-one (80 mg, 0.22 mmol) was dissolved in tetrahydrofuran (3 mL), followed by an addition of palladium-on-carbon (8 mg), and then reacted under hydrogen atmosphere for 1 hour. After the reaction was completed, the filtrate was dried over anhydrous sodium sulfate, filtered and concentrated to obtain a crude product of the title compound (49 mg, 0.17 mmol), which was used directly in the next step without purification.

Its structural characterization data are as follows:

ESI-MS (m/z): 279.2 [M+H]⁺

### Step 6: Preparation of tert-butyl (4-((S)-8-((5-(((2R,11aS)-2,7-dimethoxy-5-oxo-2,3,5, 10,11,11a-hexahydro-1H)-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (7-26-7)

Tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (30.8 mg, 0.053 mmol) and (2R,11aS)-8-hydroxy-2,7-dimethoxy-1,2,3,10,11,11a-hexahydro-5H-benzo[e]pyrrolo[1,2-a][1,4]diazepin-5-one (15 mg, 0.05 mmol) were dissolved in DMF (1.5 mL), followed by an addition of potassium carbonate (13.8 mg, 0.1 mmol), and reacted under stirring for 16 hours. The reaction was quenched with water, and the reaction mixture was extracted with ethyl acetate 3 times (10 mL×3). The organic phases were combined, washed with saturated brine, dried, concentrated, purified by silica gel column (MeOH/DCM=0% to 5%) and concentrated again to obtain the title compound (19.6 mg, 0.25 mmol).

Its structural characterization data are as follows:

ESI-MS (m/z): 784.4 [M+H]⁺

### Step 7: Preparation of (S)-2-(4-aminophenyl)-7-methoxy-8-((5-(((2S,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-25)

Tert-butyl (4-((S)-8-((5-(((2R,11aS)-2,7-dimethoxy-5-oxo-2,3,5,10,11,11a-hexahydro-1H)-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (19.6 mg, 0.25 mmol) was dissolved in dichloromethane (2 mL), followed by a dropwise addition of trifluoroacetic acid (1 mL) under stirring. After the addition was complete, the reaction mixture was stirred continuously for 1 hour, concentrated under reduced pressure, purified by reverse phase column chromatography (acetonitrile/1% formic acid aqueous solution=0% to 70%) and then freeze-dried to obtain the title compound (9.2 mg, 13.16 μmol).

Its structural characterization data are as follows:

ESI-MS (m/z): 684.4 [M+H]⁺

¹H NMR (400 MHz, DMSO): δ 7.37 (s, 1H), 7.29 (s, 1H), 7.26 (s, 1H), 7.11 (d, J=8.4 Hz, 2H), 6.53 (d, J=8.4 Hz, 2H), 6.30 (s, 1H), 6.26 (s, 1H), 5.19 (s, 2H), 4.19-4.08 (m, 1H), 3.96-3.84 (m, 5H), 3.74-3.68 (m, 2H), 3.66 (s, 3H), 3.64 (s, 3H), 3.56-3.46 (m, 3H), 3.29-3.22 (m, 2H), 3.22 (s, 3H), 3.02-2.93 (m, 1H), 2.73-2.66 (m, 1H), 1.85-1.76 (m, 4H), 1.75-1.66 (m, 1H), 1.61-1.50 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 11: Preparation of (S)-2-(4-aminophenyl)-8-((5-(((2S,11aS)-2,7-dimethoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-26)

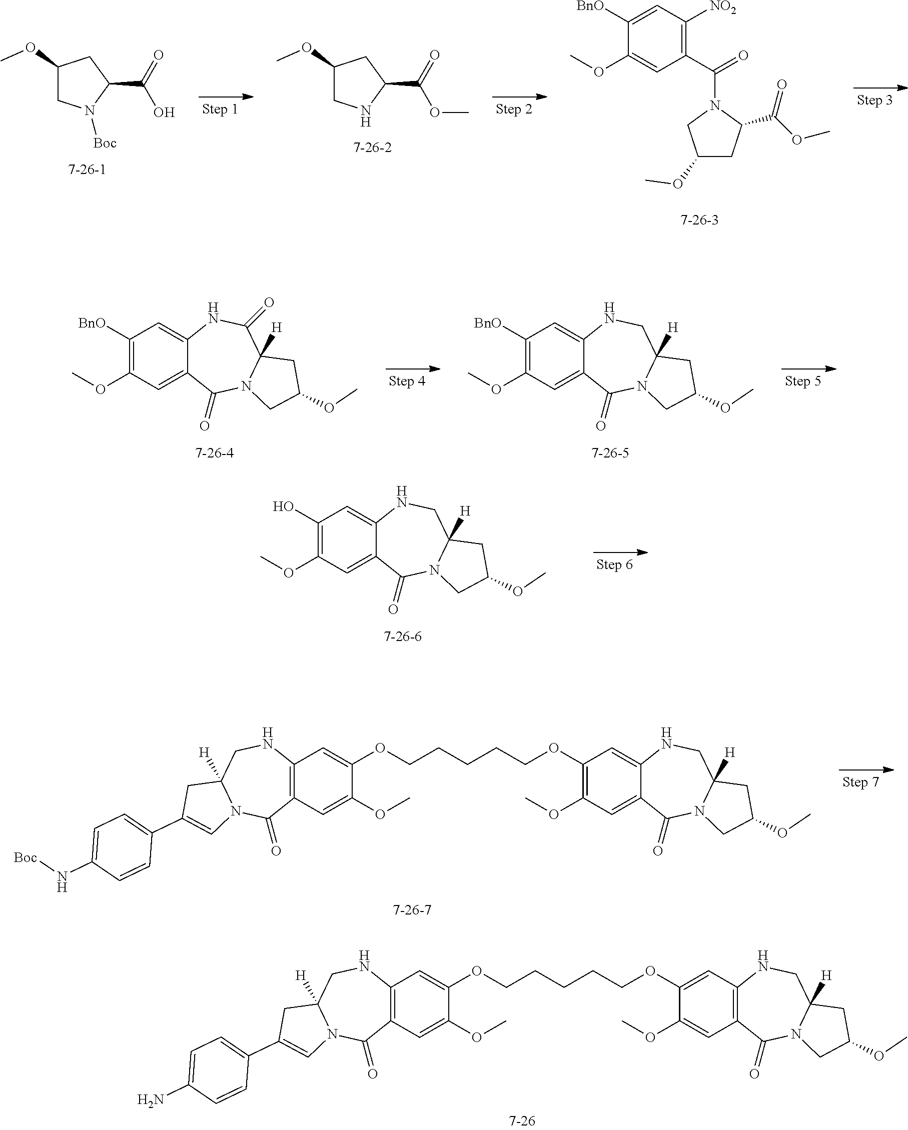

### Step 1: Preparation of methyl (2S,4S)-4-(methoxy)pyrrolidine-2-carboxylate (7-26-2)

1-(Tert-butyl)2-methyl(2S,4S)-4-(methoxy)pyrrolidine-1,2-dicarboxylic acid (1.0 g, 4.08 mmol) was dissolved in methanol (10 mL), followed by an addition of HCl (4M in EA) (5 mL), and reacted at room temperature for 1 hour. The reaction mixture was concentrated to obtain a crude product, which was used directly in the next step without purification (797.66 mg, 4.08 mmol).

Its structural characterization data are as follows:

ESI-MS (m/z): 196.2 [M+H]⁺

### Step 2: Preparation of methyl (2S,4S)-1-(4-(benzyloxy)-5-methoxy-2-nitrobenzoyl)-4-(methoxy)pyrrolidine-2-carboxylate (7-26-3)

Methyl (2S,4S)-4-(methoxy)pyrrolidine-2-carboxylate (797.66 mg, 4.08 mmol) and 4-(benzyloxy)-5-methoxy-2-nitrobenzoic acid (1.24 g, 4.08 mmol) were dissolved in DMF (10 mL), followed by an addition of HATU (1.86 g, 4.89 mmol) and DIPEA (1.58 g, 12.23 mmol), and reacted at room temperature for 1 hour. The reaction mixture was added to ethyl acetate (100 mL) and saturated brine (100 mL), stirred, and allowed to stand for liquid separation. The organic phase was dried over anhydrous sodium sulfate, filtered and concentrated to obtain a crude product, which was purified by silica gel column (ethyl acetate/petroleum ether=20% to 60%) and concentrated again to obtain the title compound (1.8 g, 4.05 mmol).

Its structural characterization data are as follows:

ESI-MS (m/z): 445.2 [M+H]⁺

### Step 3: Preparation of (2S,11aS)-8-(benzyloxy)-7-methoxy-2-(methoxy)-1,2,3,11a-tetrahydro-5H-benzo[e]pyrrolo[1,2-a][1,4]diazepin-5,11(10H)-dione (7-26-4)

Methyl (2S,4R)-1-(4-(benzyloxy)-5-methoxy-2-nitrobenzoyl)-4-(methoxy)pyrrolidin-2-carboxylate (1.8 g, 4.05 mmol) was dissolved in methanol (30 mL), followed by an addition of zinc powder (2.63 g, 40.50 mmol) and saturated aqueous ammonium chloride solution (40 mL), heated to 85° C. and reacted under refluxing for 16 hours. The reaction mixture was added to ethyl acetate (150 mL) and saturated brine (100 mL), stirred, and allowed to stand for liquid separation. The organic phase was dried over anhydrous sodium sulfate and concentrated to obtain a crude product of the title compound (1.5 g, 3.92 mmol), which was used directly in the next step without purification.

Its structural characterization data are as follows:

ESI-MS (m/z): 383.2 [M+H]⁺

### Step 4: Preparation of (2S,11aS)-8-(benzyloxy)-2,7-dimethoxy-1,2,3,10,11,11a-hexahydro-5H-benzo[e]pyrrolo[1,2-a][1,4]diazepin-5-one (7-26-5)

(2S,11aS)-8-(Benzyloxy)-2,7-dimethoxy-1,2,3,11a-tetrahydro-5H-benzo[e]pyrrolo[1,2-a][1,4]diazepin-5,11(10H)-dione (0.8 g, 2.09 mmol) was dissolved in tetrahydrofuran (10 mL), followed by an addition of sodium borohydride (158.13 mg, 41.8 mmol) and a dropwise addition of trifluoroacetic acid (596 mg, 5.23 mmol) at room temperature until no large amount of gas escaped, heated to 75° C. and reacted under refluxing for 5 hours. The reaction mixture was concentrated, followed by an addition of water (50 mL), and then extracted twice with ethyl acetate (50 mL×2). The organic phases were combined, washed with saturated sodium chloride aqueous solution, dried over anhydrous sodium sulfate, concentrated under reduced pressure, purified by reverse phase column chromatography (acetonitrile/1% formic acid aqueous solution=0% to 70%), and then freeze-dried to obtain the title compound (640 mg, 1.73 mmol).

Its structural characterization data are as follows:

ESI-MS (m/z): 369.1 [M+H]⁺

### Step 5: Preparation of (2S,11aS)-8-hydroxy-2,7-dimethoxy-1,2,3,10,11,11a-hexahydro-5H-benzo[e]pyrrolo[1,2-a][1,4]diazepin-5-one (7-26-6)

(2S,11aS)-8-(Benzyloxy)-2,7-dimethoxy-1,2,3,10,11,11a-hexahydro-5H-benzo[e]pyrrolo[1,2-a][1,4]diazepin-5-one (640 mg, 1.73 mmol) was dissolved in methanol (10 mL), followed by an addition of palladium-on-carbon (30 mg), and then reacted under hydrogen atmosphere for 1 hour. After the reaction was completed, the filtrate was dried over anhydrous sodium sulfate, filtered and concentrated to obtain a crude product of the title compound (434 mg, 1.56 mmol), which was used directly in the next step without purification.

Its structural characterization data are as follows:

ESI-MS (m/z): 279.2 [M+H]⁺

### Step 6: Preparation of tert-butyl (4-((S)-8-((5-(((2S,11aS)-2,7-dimethoxy-5-oxo-2,3,5, 10,11,11a-hexahydro-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (7-26-7)

Tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (21.07 mg, 35.93 μmol) and (2S,11aS)-8-hydroxy-2,7-dimethoxy-1,2,3,10,11,11a-hexahydro-5H-benzo[e]pyrrolo[1,2-a][1,4]diazepin-5-one (10 mg, 35.93 μmol) were dissolved in DMF (1 mL), followed by an addition of potassium carbonate (14.88 mg, 107.80 μmol), and reacted under stirring for 16 hours. The reaction was quenched with water, and the reaction mixture was extracted with ethyl acetate 3 times (10 mL×3). The organic phases were combined, washed with saturated brine, dried, concentrated, purified by silica gel column (MeOH/DCM=0% to 5%) and concentrated again to obtain the title compound (20 mg, 25.51 μmol).

Its structural characterization data are as follows:

ESI-MS (m/z): 784.4 [M+H]⁺

### Step 7: Preparation of tert-butyl (S)-2-(4-aminophenyl)-8-((5-(((2S,11aS)-2,7-dimethoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-26)

Tert-butyl (4-((S)-8-((5-(((2S,11aS)-2,7-dimethoxy-5-oxo-2,3,5,10,11,11a-hexahydro-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (20 mg, 25.51 μmol) was dissolved in dichloromethane (2 mL), followed by a dropwise addition of trifluoroacetic acid (0.5 mL) under stirring. After the addition was completed, the reaction mixture was stirred continuously for 1 hour, concentrated under reduced pressure, purified by reverse phase column chromatography (acetonitrile/1% formic acid aqueous solution=0% to 70%), and then freeze-dried to obtain the title compound (13.51 mg, 19.56 μmol).

Its structural characterization data are as follows:

ESI-MS (m/z): 684.4 [M+H]⁺

¹H NMR (400 MHz, DMSO): δ 7.39 (s, 1H), 7.28 (d, J=9.3 Hz, 2H), 6.85-6.75 (m, 2H), 6.48-6.63 (m, 2H), 6.31 (s, 1H), 6.29 (s, 1H) 4.22-4.15 (m, 1H), 3.97-3.86 (m, 4H), 3.75-3.65 (m, 2H), 3.66 (s, 3H), 3.64 (s, 3H), 3.62-3.50 (m, 6H), 3.24 (s, 3H), 3.32-3.16 (m, 2H), 2.76-2.67 (m, 1H), 2.43-2.36 (m, 1H), 1.87-1.73 (m, 4H), 1.60-1.49 (m, 4H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 12: Preparation of (S)-8-((5-(((2R,11aS)-2-amino-7-methoxy-5-oxo-2,3,5,10,11,11a-hexahydro-1H-benzo[e]pyrrolo[1,2-a][1,4]diazepin-8-yl)oxy)pentyl)oxy)-2-(4-aminophenyl)-7-methoxy-1,10,11,11a-tetrahydro-5H-benzo[e]pyrrolo[1,2-a][1,4]diazepin-5-one (7-27)

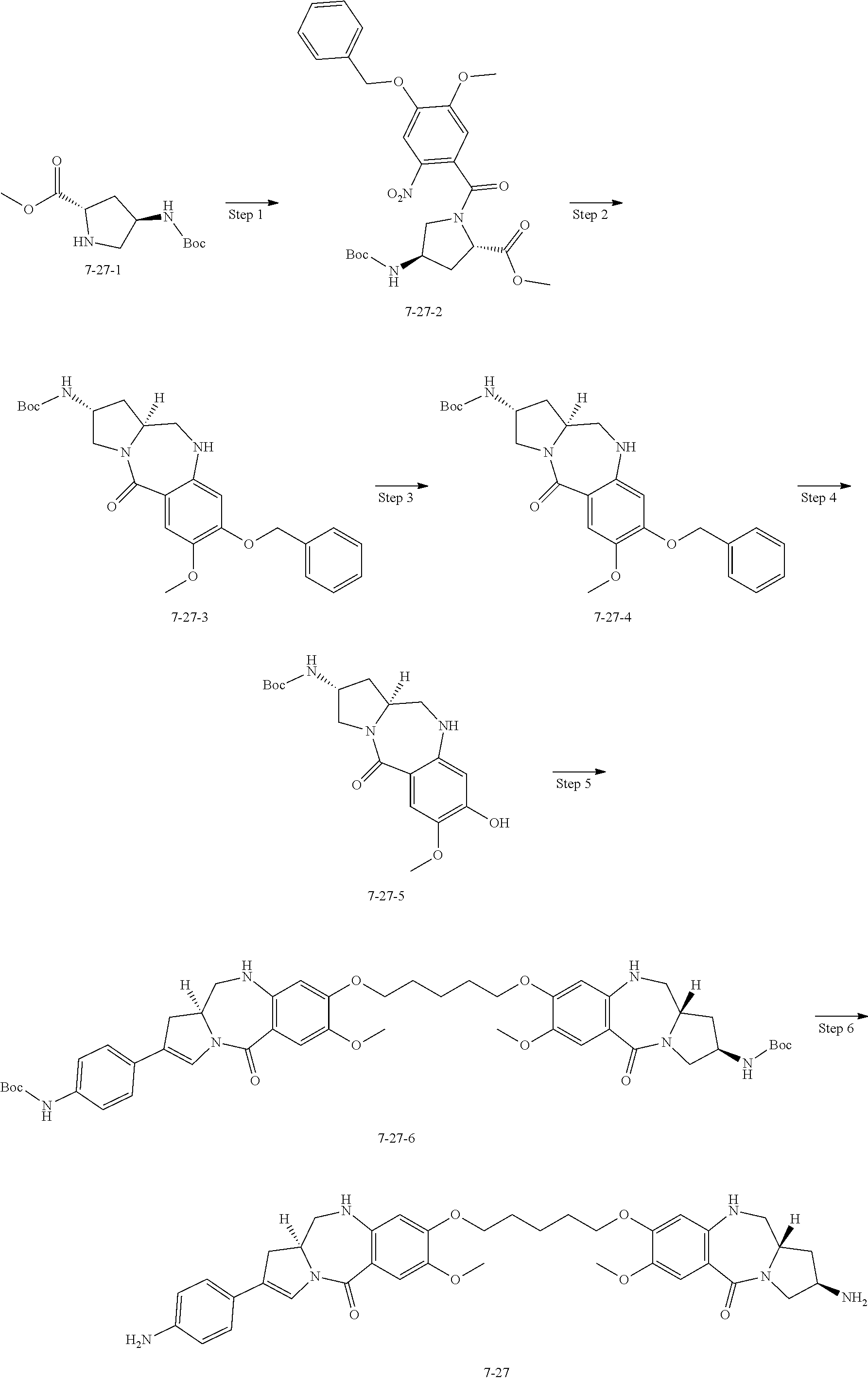

### Step 1: Preparation of methyl (2S,4R)-1-(4-(benzyloxy)-5-methoxy-2-nitrobenzoyl)-4-((tert-butoxycarbonyl)amino)pyrrolidine-2-carboxylate (7-27-2)

Methyl (2S,4R)-4-((tert-butoxycarbonyl)amino)pyrrolidine-2-carboxylate (1 g, 4.09 mmol, FR) and 4-(benzyloxy)-5-methoxy-2-nitrobenzoic acid (1.37 g, 4.50 mmol, FR) were dissolved in DMF (10 mL), followed by an addition of HATU (1.71 g, 4.50 mmol) and DIPEA (1.59 g, 12.28 mmol), and reacted at room temperature for 1 hour. The reaction mixture was added to ethyl acetate (50 mL) and saturated brine (50 mL), stirred, and allowed to stand for liquid separation. The organic phase was dried over anhydrous sodium sulfate, filtered and concentrated to obtain a crude product, which was purified by silica gel column (ethyl acetate/petroleum ether=20% to 60%) and concentrated again to obtain the title compound (2.1 g, 3.97 mmol).

Its structural characterization data are as follows:

ESI-MS (m/z): 530.2 [M+H]⁺

### Step 2: Preparation of tert-butyl ((2R,11aS)-8-(benzyloxy)-7-methoxy-5,11-dioxo-2,3,5, 10,11,11a-hexahydro-1H-benzo[e]pyrrolo[1,2-a][1,4]diazepin-2-yl)carbamate (7-27-3)

Methyl (2S,4R)-1-(4-(benzyloxy)-5-methoxy-2-nitrobenzoyl)-4-((tert-butoxycarbonyl) amino)pyrrolidine-2-carboxylate (2.1 g, 3.97 mmol, FR) was dissolved in methanol (50 mL), followed by an addition of zinc powder (2.58 g, 15.86 mmol) and a saturated aqueous solution of ammonium chloride (20 mL), heated to 85° C. and reacted under refluxing for 16 hours. The reaction mixture was added to ethyl acetate (150 mL) and saturated brine (100 mL), stirred, and allowed to stand for liquid separation. The organic phase was dried over anhydrous sodium sulfate and concentrated to obtain a crude product of the title compound (1.8 g, 3.85 mmol), which was used directly in the next step without purification.

Its structural characterization data are as follows:

ESI-MS (m/z): 468.3 [M+H]⁺

### Step 3: Preparation of tert-butyl ((2R,11aS)-8-(benzyloxy)-7-methoxy-5-oxo-2,3,5, 10,11,11a-hexahydro-1H-benzo[e]pyrrolo[1,2-a][1,4]diazepin-2-yl)carbamate (7-27-4)

Tert-butyl ((2R,11aS)-8-(benzyloxy)-7-methoxy-5,11-dioxo-2,3,5,10,11,11a-hexahydro-1H-benzo[e]pyrrolo[1,2-a][1,4]diazepin-2-yl)carbamate (1.8 g, 3.85 mmol, FR) was dissolved in tetrahydrofuran (100 mL), followed by an addition of sodium borohydride (843.15 mg, 11.55 mmol) and a dropwise addition of trifluoroacetic acid (877.8 mg, 7.7 mmol) at room temperature until no large amount of gas was released. The reaction mixture was then heated to 75° C. and reacted under refluxing for 5 hours. The reaction mixture was concentrated, followed by an addition of water (50 mL), and then extracted twice with ethyl acetate (50 mL×2). The organic phases were combined, washed with saturated sodium chloride aqueous solution, dried over anhydrous sodium sulfate, filtered and concentrated to obtain a crude product of the title compound (1.4 g, 3.09 mmol), which was used directly in the next step without purification.

Its structural characterization data are as follows:

ESI-MS (m/z): 454.2 [M+H]⁺

### Step 4: Preparation of tert-butyl ((2R,11aS)-8-hydroxy-7-methoxy-5-oxo-2,3,5,10,11,11a-hexahydro-1H-benzo[e]pyrrolo[1,2-a][1,4]diazepin-2-yl)carbamate (7-27-5)

Tert-butyl ((2R,11aS)-8-(benzyloxy)-7-methoxy-5-oxo-2,3,5,10,11,11a-hexahydro-1H-benzo[e]pyrrolo[1,2-a][1,4]diazepin-2-yl)carbamate (100 mg, 220.50 μmol, FR) was dissolved in methanol (5 mL), followed by an addition of palladium-on-carbon (5 mg), and reacted under hydrogen atmosphere for 1 hour. After the reaction was completed, the reaction mixture was filtered, and the filtrate was dried over anhydrous sodium sulfate, filtered and concentrated to obtain a crude product of the title compound (80.13 mg, 220.50 μmol), which was used directly in the next step without purification.

Its structural characterization data are as follows:

ESI-MS (m/z): 364.2 [M+H]⁺

### Step 5: Preparation of tert-butyl (4-((S)-8-((5-(((2R,11aS)-2-((tert-butyloxycarbonyl) amino)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (7-27-6)

Tert-butyl ((2R,11aS)-8-hydroxy-7-methoxy-5-oxo-2,3,5,10,11,11a-hexahydro-1H-benzo[e]pyrrolo[1,2-a][1,4]diazepin-2-yl)carbamate (10 mg, 27.52 μmol) and tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (16.14 mg, 27.52 μmol) were dissolved in DMF (1 mL), followed by an addition of potassium carbonate (11.39 mg, 82.55 μmol), and reacted under stirring for 16 hours. The reaction was quenched with water, and the reaction mixture was extracted with ethyl acetate 3 times (10 mL×3). The organic phases were combined, washed with saturated brine, dried, concentrated, and purified by silica gel column (MeOH/DCM=0% to 5%) to obtain the title compound (20 mg, 23.01 μmol).

Its structural characterization data are as follows:

ESI-MS (m/z): 869.8 [M+H]⁺

### Step 6: Preparation of (S)-8-((5-(((2R,11aS)-2-amino-7-methoxy-5-oxo-2,3,5,10,11,11a-hexahydro-1H-benzo[e]pyrrolo[1,2-a][1,4]diazepin-8-yl)oxy)pentyl)oxy)-2-(4-aminophenyl)-7-methoxy-1,10,11,11a-tetrahydro-5H-benzo[e]pyrrolo[1,2-a][1,4]diazepin-5-one (7-27)

Tert-butyl (4-((S)-8-((5-(((2R,11aS)-2-((tert-butyloxycarbonyl)amino)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (20 mg, 23.01 μmol) was dissolved in dichloromethane (2 mL), followed by a dropwise addition of trifluoroacetic acid (0.5 mL) under stirring. After the addition was complete, the reaction mixture was stirred continuously for 1 hour, concentrated under reduced pressure, purified by reverse phase column chromatography (acetonitrile/1% formic acid aqueous solution=0% to 70%) and then freeze-dried to obtain the title compound (5.41 mg, 0.045 mmol).

Its structural characterization data are as follows:

ESI-MS (m/z): 670.4 [M+H]⁺

¹H NMR (400 MHz, DMSO): δ 7.36 (s, 1H), 7.29 (s, 1H), 7.25 (s, 1H), 7.11 (d, J=8.5 Hz, 2H), 6.53 (d, J=8.6 Hz, 2H), 6.29 (d, J=9.3 Hz, 2H), 4.17-4.09 (m, 1H), 3.96-3.90 (m, 4H), 3.65 (d, J=7.1 Hz, 6H), 3.51 (d, J=4.9 Hz, 2H), 3.30-3.20 (m, 4H), 2.99 (dd, J=12.9, 8.8 Hz, 2H), 2.23-2.14 (m, 2H), 1.96-1.87 (m, 2H), 1.84-1.76 (m, 4H), 1.56 (d, J=6.4 Hz, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 13: Preparation of (S)-2-(4-aminophenyl)-8-((5-(((S)-2,2-difluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-29)

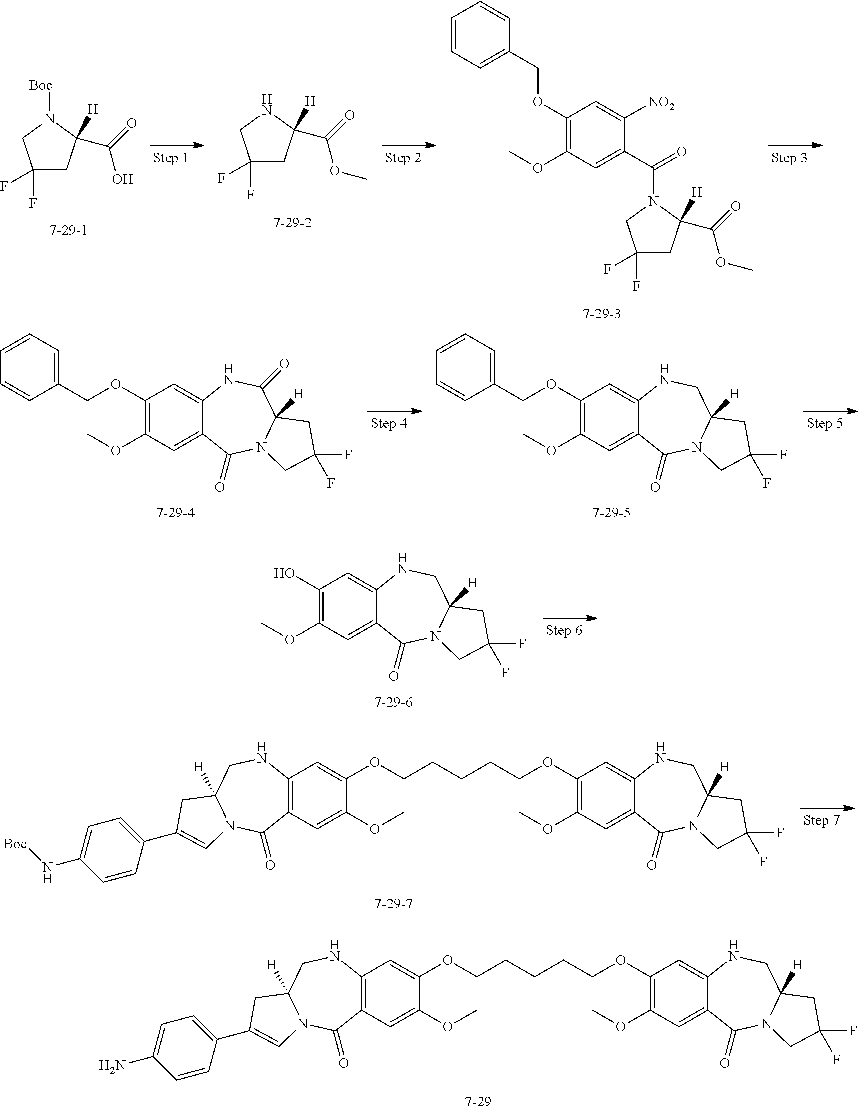

### Step 1: Preparation of methyl (S)-4,4-difluoropyrrolidine-2-carboxylate (7-29-2)

(S)-1-(Tert-butyloxycarbonyl)-4,4-difluoropyrrolidine-2-carboxylic acid (0.6 g, 2.39 mmol) was dissolved in MeOH (3 mL), followed by a dropwise addition of 4M HCl/dioxane solution (4 mL), and reacted under stirring at room temperature for 20 hours. After the reaction was completed, the reaction mixture was directly concentrated under reduced pressure to obtain a crude hydrochloride of the title compound (460 mg, 2.28 mmol), which was directly used in the next step without purification.

Its structural characterization data are as follows:

ESI-MS (m/z): 166.1 [M+H]⁺

### Step 2: Preparation of methyl (S)-1-(4-(benzyloxy)-5-methoxy-2-nitrobenzoyl)-4,4-difluoropyrrolidine-2-carboxylate (7-29-3)

4-(Benzyloxy)-5-methoxy-2-nitrobenzoic acid (743.76 mg, 2.45 mmol) was dissolved in DMF (5 mL), followed by an addition of HATU (1.03 g, 2.70 mmol) and DIPEA (316.96 mg, 2.45 mmol), and stirred at room temperature for 10 minutes. Another reaction bottle was taken, in which methyl (S)-4,4-difluoropyrrolidine-2-carboxylate hydrochloride (450 mg, 2.45 mmol) was dissolved in DMF (5 mL), followed by an addition of DIPEA (316.96 mg, 2.45 mmol) to make it alkaline. Then the two reaction mixtures were combined, and reacted under stirring at room temperature for 2 hours. After the reaction was completed, 30 mL of water was added to the reaction mixture, and the mixture was extracted with 15 mL of ethyl acetate 3 times. The organic phases were combined and washed with 15 mL of saturated brine, dried over anhydrous sodium sulfate, and concentrated under reduced pressure to obtain a crude product of the title compound (1.4 g, 2.18 mmol), which was directly used in the next step without purification.

Its structural characterization data are as follows:

ESI-MS (m/z): 468.2 [M+H]⁺

### Step 3: Preparation of (S)-8-(benzyloxy)-2,2-difluoro-7-methoxy-1,2,3,11a-tetrahydro-5H-benzo[e]pyrrolo[1,2-a][1,4]diazepin-5,11(10H)-dione (7-29-4)

Methanol (12 mL) and saturated aqueous ammonium chloride solution (3 mL) were added to methyl (S)-1-(4-(benzyloxy)-5-methoxy-2-nitrobenzoyl)-4,4-difluoropyrrolidine-2-carboxylate (700 mg, 1.09 mmol), and the system was not dissolved clearly. Then zinc powder (711.41 mg, 10.88 mmol) was added to the system, which was directly placed in an oil bath preheated to 75° C., and reacted for 16 hours. After the reaction was completed, the reaction mixture was cooled to room temperature, the insoluble matter was removed by filtration, the filter cake was washed with an appropriate amount of methanol, and the filtrate was concentrated under reduced pressure. 10 mL of water was added to the residue, which was then extracted with 10 mL of ethyl acetate 3 times. The organic phases were combined and washed with 10 mL of brine, dried over anhydrous sodium sulfate, and concentrated under reduced pressure to obtain a crude product of the title compound (580 mg, 1.05 mmol), which was directly used in the next step without purification.

Its structural characterization data are as follows:

ESI-MS (m/z): 389.2 [M+H]⁺

### Step 4: Preparation of (S)-8-(benzyloxy)-2,2-difluoro-7-methoxy-1,2,3,10,11,11a-hexahydro-5H-benzo[e]pyrrolo[1,2-a][1,4]diazepin-5-one (7-29-5)

(S)-8-(Benzyloxy)-2,2-difluoro-7-methoxy-1,2,3,11-tetrahydro-5H-benzo[e]pyrrolo[1,2-a][1,4]diazepin-5,11(10H)-dione (100 mg, 257.49 μmol) was dissolved in THF (2 mL), cooled to 0° C. under nitrogen protection, followed by a dropwise addition of borane (1 M in THF, 2.57 mL). After the addition, the reaction mixture was naturally warmed to room temperature and reacted for 1.5 hours. After the reaction was completed, the reaction mixture was cooled to 0° C., treated with an appropriate amount of methanol to quench the reaction, and stirred at room temperature for 1 hour. 10 mL of water was added to the reaction solution, ethyl acetate (10 mL×3) was added for extraction. The organic phases were combined and washed with 10 mL of brine, dried over anhydrous sodium sulfate, and concentrated under reduced pressure. The resultant crude product was purified by silica gel column chromatography (ethyl acetate-petroleum ether=10% to 70%) and concentrated under reduced pressure again to obtain the title compound (23 mg, 61.43 μmol).

Its structural characterization data are as follows:

ESI-MS (m/z): 385.1 [M+H]⁺

### Step 5: Preparation of (S)-2,2-difluoro-8-hydroxy-7-methoxy-1,2,3,10,11,11a-hexahydro-5H-benzo[e]pyrrolo[1,2-a][1,4]diazepin-5-one (7-29-6)

(S)-8-(Benzyloxy)-2,2-difluoro-7-methoxy-1,2,3,10,11,11a-hexahydro-5H-benzo[e]pyrrolo[1,2-a][1,4]diazepin-5-one (23 mg, 61.43 μmol) was suspended in methanol (2 mL), followed by an addition of ethyl acetate (1 mL) to completely dissolve the raw material and Pd/C (7.46 mg, 6.14 μmol, 10% purity), and subjected to hydrogen replacement for three times. The reaction was carried out under stirring and a hydrogen balloon atmosphere at room temperature for 3 hours. After the reaction was completed, the reaction mixture was filtered, the filter cake was washed with an appropriate amount of ethyl acetate, and the filtrate was directly concentrated under reduced pressure to obtain a crude product of the title compound (23 mg, 59.88 μmol), which was directly used in the next step without purification.

Its structural characterization data are as follows:

ESI-MS (m/z): 285.2[M+H]⁺

### Step 6: Preparation of tert-butyl (4-((S)-8-((5-(((S)-2,2-difluoro-7-methoxy-5-oxo-2,3,5, 10,11,11a-hexahydro)-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (7-29-7)

Tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (20.63 mg, 35.18 μmol) and (S)-2,2-difluoro-8-hydroxy-7-methoxy-1,2,3,10,11,11a-hexahydro-5H-benzo[e]pyrrolo[1,2-a][1,4]diazepin-5-one (10 mg, 35.18 μmol) were dissolved in DMF (0.5 mL), followed by an addition of K₂CO₃ (14.59 mg, 105.54 μmol), and stirred at room temperature for 18 hours. After the reaction was completed, 4 mL of water was added to the reaction solution, and extracted with ethyl acetate (5 mL×3). The organic phases were combined and washed with 5 mL of saturated brine, dried over anhydrous sodium sulfate, and concentrated under reduced pressure to obtain a crude product of the title compound (28 mg, 26.59 μmol), which was used directly in the next step without purification.

Its structural characterization data are as follows:

ESI-MS (m/z): 790.4 [M+H]⁺

### Step 7: Preparation of (S)-2-(4-aminophenyl)-8-((5-(((S)-2,2-difluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-29)

Dichloromethane (1 mL) was added to tert-butyl (4-((S)-8-((5-(((S)-2,2-difluoro-7-methoxy-5-oxo-2,3,5,10,11,11a-hexahydro)-1H-benzo[e]pyrrolo[1,2-a][1,4]diazepin-8-yl)oxy)pentyl)oxy)-7-methoxy-5-oxo-5,10,11,11a-tetrahydro-1H-benzo[e]pyrrolo[1,2-a][1,4]diazepin-2-yl)phenyl)carbamate (28 mg, 35.45 μmol) at room temperature, followed by an addition of TFA (0.2 mL). After the addition was completed, the reaction was carried out under stirring at room temperature for 2 hours. After the reaction was completed, the reaction mixture was directly concentrated under reduced pressure to remove the solvent, and the residue was directly purified by preparative HPLC and freeze-dried to obtain the title compound (4.5 mg, 6.29 μmol).

Its structural characterization data are as follows:

ESI-MS (m/z): 690.4 [M+H]⁺; 345.9 [M/2+1]⁺

¹H NMR (400 MHz, DMSO-d6) δ 7.33 (s, 1H), 7.29 (s, 1H), 7.26 (s, 1H), 7.11 (d, J=8.4 Hz, 1H), 6.53 (d, J=8.4 Hz, 2H), 6.49 (brs, 1H), 6.30 (s, 2H), 5.19 (brs, 1H), 4.14-4.10 (m, 1H), 3.98-3.84 (m, 7H), 3.67 (s, 3H), 3.64 (s, 3H), 3.57-3.50 (m, 2H), 3.25-3.14 (m, 3H), 2.75-2.72 (m, 2H), 1.82-1.78 (m, 3H), 1.82-1.78 (m, 4H), 1.59-1.53 (m, 2H).

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 20 | 80 | 30 |
| 2.00 | 20 | 80 | 30 |
| 18.00 | 90 | 10 | 30 |

### Preparation Example 14: Preparation of (S)-2-(4-aminophenyl)-8-((5-(((2S,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-30)

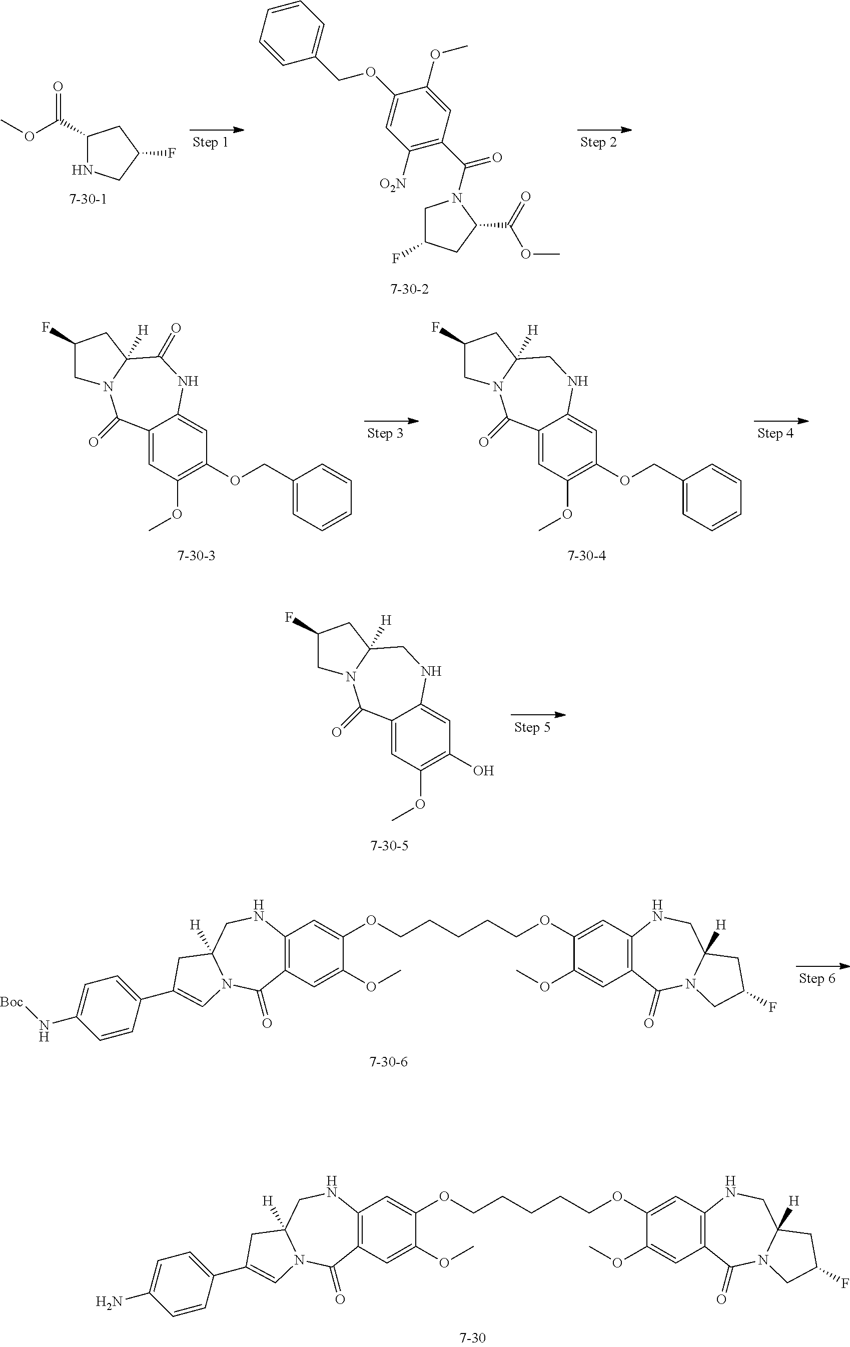

### Step 1: Preparation of methyl (2S,4S)-1-(4-(benzyloxy)-5-methoxy-2-nitrobenzoyl)-4-fluoropyrrolidin-2-carboxylate (7-30-2)

Methyl (2S,4S) 4-fluoropyrrolidin-2-carboxylate (1.07 g, 7.25 mmol) and 4-(benzyloxy)-5-methoxy-2-nitrobenzoic acid (2 g, 6.59 mmol) were dissolved in DMF (10 mL), followed by an addition of HATU (2.76 g, 7.25 mmol) and DIPEA (2.56 g, 19.78 mmol), and reacted at room temperature for 1 hour. The reaction mixture was added to ethyl acetate (100 mL) and saturated brine (100 mL), stirred, and allowed to stand for liquid separation. The organic phase was dried over anhydrous sodium sulfate, filtered, and concentrated to obtain a crude product, which was purified by silica gel column (ethyl acetate/petroleum ether=20% to 60%) and concentrated again to obtain the title compound (2.3 g, 5.32 mmol).

Its structural characterization data are as follows:

ESI-MS (m/z): 433.2 [M+H]⁺

### Step 2: Preparation of (2S,11aS)-8-(benzyloxy)-2-fluoro-7-methoxy-1,2,3,11a-tetrahydro-5H-benzo[e]pyrrolo[1,2-a][1,4]diazepin-5,11(10H)-dione (7-30-3)

Methyl (2S,4S)-1-(4-(benzyloxy)-5-methoxy-2-nitrobenzoyl)-4-fluoropyrrolidin-2-carboxylate (2.3 g, 5.32 mmol) was dissolved in methanol (50 mL), followed by an addition of zinc powder (3.46 g, 53.19 mmol) and a saturated aqueous solution of ammonium chloride (10 mL), heated to 85° C. and reacted under refluxing for 16 hours. The reaction mixture was added to ethyl acetate (150 mL) and saturated brine (100 mL), stirred, and allowed to stand for liquid separation. The organic phase was dried over anhydrous sodium sulfate and concentrated to obtain a crude product of the title compound (1.8 g, 4.86 mmol), which was used directly in the next step without purification.

Its structural characterization data are as follows:

ESI-MS (m/z): 371.2 [M+H]⁺

### Step 3: Preparation of (2S,11aS)-8-(benzyloxy)-2-fluoro-7-methoxy-1,2,3,10,11,11a-hexahydro-5H-benzo[e]pyrrolo[1,2-a][1,4]diazepin-5-one (7-30-4)

(2S,11aS)-8-(Benzyloxy)-2-fluoro-7-methoxy-1,2,3,11a-tetrahydro-5H-benzo[e]pyrrolo[1,2-a][1,4]diazepin-5,11(10H)-dione (1.8 g, 4.86 mmol) was dissolved in tetrahydrofuran (100 mL), followed by an addition of sodium borohydride (1064.34 mg, 14.58 mmol) and a dropwise addition of trifluoroacetic acid (1108.1 mg, 9.72 mmol) at room temperature until no large amount of gas released, heated to 75° C. and reacted under refluxing for 5 hours. The reaction mixture was concentrated, followed by an addition of water (50 mL), and then extracted twice with ethyl acetate (50 mL×2). The organic phases were combined and washed with saturated sodium chloride aqueous solution, dried over anhydrous sodium sulfate, filtered and concentrated to obtain a crude product of the title compound (1.4 g, 3.93 mmol), which was used directly in the next step without purification.

Its structural characterization data are as follows:

ESI-MS (m/z): 357.3 [M+H]⁺

### Step 4: Preparation of (2S,11aS)-2-fluoro-8-hydroxy-7-methoxy-1,2,3,10,11,11a-hexahydro-5H-benzo[e]pyrrolo[1,2-a][1,4]diazepin-5-one (7-30-5)

(2S,11aS)-8-(Benzyloxy)-2-fluoro-7-methoxy-1,2,3,10,11,11a-hexahydro-5H-benzo[e]pyrrolo[1,2-a][1,4]diazepin-5-one (200 mg, 561.18 μmol) was dissolved in methanol (10 mL), followed by an addition of palladium-on-carbon (5 mg), and reacted under a hydrogen atmosphere for 1 hour. After the reaction was completed, the reaction mixture was filtered, and the filtrate was dried over anhydrous sodium sulfate, filtered and concentrated to obtain a crude product of the title compound (0.14 g, 525.79 μmol), which was used directly in the next step without purification.

Its structural characterization data are as follows:

ESI-MS (m/z): 267.1 [M+H]⁺

### Step 5: Preparation of (S)-2-(4-aminophenyl)-8-((5-(((2S,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-30-6)

(2S,11aS)-2-Fluoro-8-hydroxy-7-methoxy-1,2,3,10,11,11a-hexahydro-5H-benzo[e]pyrrolo[1,2-a][1,4]diazepin-5-one (10 mg, 37.56 μmol) and tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (22.03 mg, 37.56 μmol) were dissolved in DMF (1 mL), followed by an addition of potassium carbonate (15.55 mg, 112.67 μmol), and reacted under stirring for 16 hours. The reaction was quenched with water, and the reaction mixture was extracted with ethyl acetate for 3 times (10 mL×3). The organic phases were combined, washed with saturated brine, dried, concentrated, purified by silica gel column (MeOH/DCM=0% to 5%) and concentrated again to obtain the title compound (20 mg, 25.91 μmol).

Its structural characterization data are as follows:

ESI-MS (m/z): 772.3 [M+H]⁺

### Step 6: Preparation of (S)-2-(4-aminophenyl)-8-((5-(((2S,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-30)

(S)-2-(4-Aminophenyl)-8-((5-(((2S,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (20 mg, 25.91 μmol) was dissolved in dichloromethane (2 mL), followed by a dropwise addition of trifluoroacetic acid (0.5 mL) under stirring. After the addition was completed, the reaction mixture was stirred continuously for 1 hour, concentrated under reduced pressure, purified by reverse phase column chromatography (acetonitrile/1% formic acid aqueous solution=0% to 70%) and then freeze-dried to obtain the title compound (8.14 mg, 12.00 μmol).

Its structural characterization data are as follows:

ESI-MS (m/z): 673.3 [M+H]⁺

¹H NMR (400 MHz, DMSO) δ 7.40 (s, 1H), 7.29 (s, 1H), 7.24 (s, 1H), 6.87-6.70 (m, 2H), 6.60-6.45 (m, 2H), 6.31 (s, 2H), 4.17 (s, 1H), 4.03-3.87 (m, 4H), 3.87-3.76 (m, 2H), 3.67 (s, 3H), 3.65 (s, 3H), 3.58-3.45 (m, 4H), 3.29-3.20 (m, 2H), 2.82-2.61 (m, 2H), 2.20-2.06 (m, 2H), 1.86-1.73 (m, 4H), 1.60-1.53 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 15: Preparation of (S)-2-(4-aminophenyl)-8-((5-(((2R,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-31)

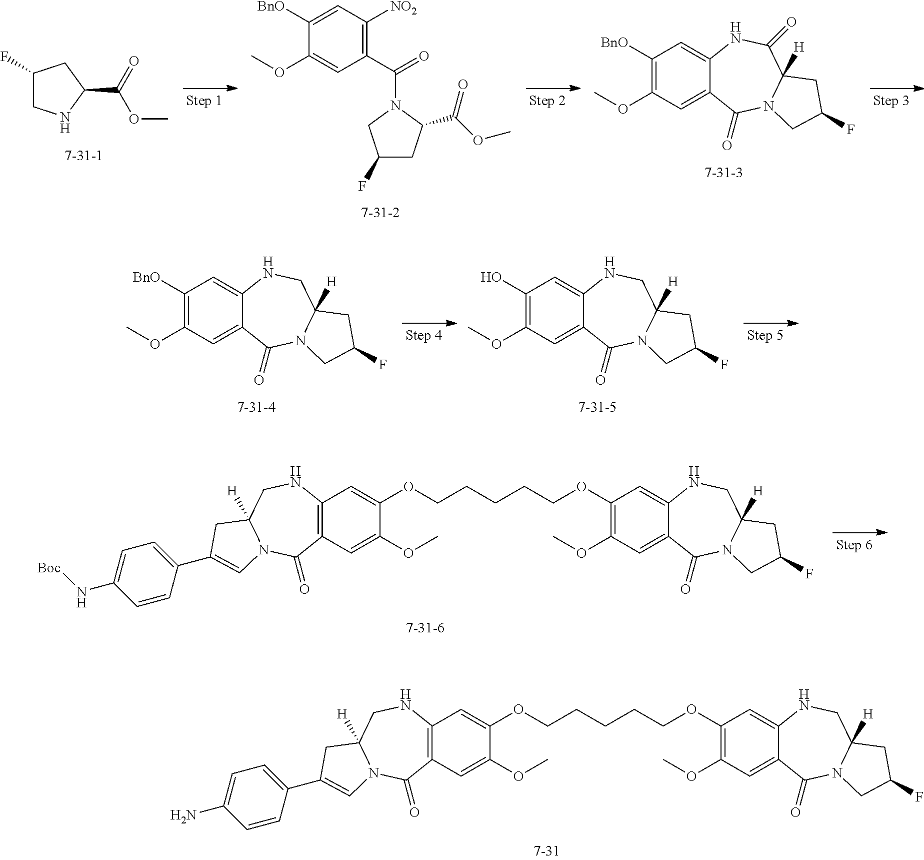

### Step 1: Preparation of methyl (2R,4S)-1-(4-(benzyloxy)-5-methoxy-2-nitrobenzoyl)-4-fluoropyrrolidin-2-carboxylate (7-31-2)

Methyl (2R,4S)-4-fluoropyrrolidine-2-carboxylate (1.1 g, 7.25 mmol, FR) and 4-(benzyloxy)-5-methoxy-2-nitrobenzoic acid (2.05 g, 6.59 mmol, FR) were dissolved in DMF (10 mL), followed by an addition of HATU (2.76 g, 7.25 mmol) and DIPEA (2.56 g, 19.78 mmol), and reacted at room temperature for 1 hour. The reaction mixture was added to ethyl acetate (100 mL) and saturated brine (100 mL), stirred, and allowed to stand for liquid separation. The organic phase was dried over anhydrous sodium sulfate, filtered, and concentrated to obtain a crude product, which was purified by silica gel column (ethyl acetate/petroleum ether=20% to 60%) and concentrated again to obtain the title compound (2.1 g, 4.85 mmol).

Its structural characterization data are as follows:

ESI-MS (m/z): 433.2 [M+H]⁺

### Step 2: Preparation of (2R,11aS)-8-(benzyloxy)-2-fluoro-7-methoxy-1,2,3,11a-tetrahydro-5H-benzo[e]pyrrolo[1,2-a][1,4]diazepin-5,11(10H)-dione (7-31-3)

Methyl (2R,4S)-1-(4-(benzyloxy)-5-methoxy-2-nitrobenzoyl)-4-fluoropyrrolidin-2-carboxylate (2.1 g, 4.85 mmol) was dissolved in methanol (50 mL), followed by an addition of zinc powder (3.4 g, 53.1 mmol) and a saturated aqueous solution of ammonium chloride (10 mL), heated to 85° C. and reacted under refluxing for 16 hours. The reaction mixture was added to ethyl acetate (150 mL) and saturated brine (100 mL), stirred, and allowed to stand for liquid separation. The organic phase was dried over anhydrous sodium sulfate and concentrated to obtain a crude product of the title compound (1.7 g, 4.7 mmol), which was used directly in the next step without purification.

Its structural characterization data are as follows:

ESI-MS (m/z): 371.2 [M+H]⁺

### Step 3: Preparation of (2R,11aS)-8-(benzyloxy)-2-fluoro-7-methoxy-1,2,3,10,11,11a-hexahydro-5H-benzo[e]pyrrolo[1,2-a][1,4]diazepin-5-one (7-31-4)

(2R,11aS)-8-(Benzyloxy)-2-fluoro-7-methoxy-1,2,3,11a-tetrahydro-5H-benzo[e]pyrrolo[1,2-a][1,4]diazepin-5,11(10H)-dione (400 mg, 1.04 mmol) was dissolved in tetrahydrofuran (5 mL), and cooled to 0° C., followed by a dropwise addition of tetrahydrofuran borane complex (10.3 mL). After the addition was completed, the reaction mixture was heated to room temperature and reacted for 3 hours. Methanol (10 ml) was added to the reaction mixture, and the mixture was then concentrated to obtain a crude product of the title compound, which was purified by silica gel column (ethyl acetate/petroleum ether=0% to 70%) and concentrated to obtain the title compound (110 mg, 0.31 mmol).

Its structural characterization data are as follows:

ESI-MS (m/z): 357.3 [M+H]⁺

### Step 4: Preparation of (2R,11aS)-2-fluoro-8-hydroxy-7-methoxy-1,2,3,10,11,11a-hexahydro-5H-benzo[e]pyrrolo[1,2-a][1,4]diazepin-5-one (7-31-5)

(2S,11aS)-8-(Benzyloxy)-2-fluoro-7-methoxy-1,2,3,10,11,11a-hexahydro-5H-benzo[e]pyrrolo[1,2-a][1,4]diazepin-5-one (110 mg, 0.31 mmol) was dissolved in tetrahydrofuran (5 mL), followed by an addition of palladium-on-carbon (10 mg), and reacted under a hydrogen atmosphere for 1 hour. After the reaction was completed, the reaction mixture was filtered through diatomaceous earth, and the filtrate was dried over anhydrous sodium sulfate, filtered and concentrated to obtain a crude product of the title compound (50 mg, 190.1 μmol), which was used directly in the next step without purification.

Its structural characterization data are as follows:

ESI-MS (m/z): 267.1 [M+H]⁺

### Step 5: Preparation of (S)-2-(4-aminophenyl)-8-((5-(((2R,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (7-31-6)

(2R,11aS)-2-Fluoro-8-hydroxy-7-methoxy-1,2,3,10,11,11a-hexahydro-5H-benzo[e]pyrrolo[1,2-a][1,4]diazepin-5-one (50 mg, 190.1 μmol) and tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (117.3 mg, 199.5 μmol) were dissolved in DMF (3 mL), followed by an addition of potassium carbonate (52.4 mg, 380 μmol), and reacted under stirring for 16 hours. The reaction was quenched with water, and the reaction mixture was extracted with ethyl acetate for 3 times (20 mL×3). The organic phases were combined, washed with saturated brine, dried, concentrated, purified by silica gel column (MeOH/DCM=0% to 5%) and concentrated again to obtain the title compound (88 mg, 114.1 μmol).

Its structural characterization data are as follows:

ESI-MS (m/z): 772.3 [M+H]⁺

### Step 6: Preparation of (S)-2-(4-aminophenyl)-8-((5-(((2R,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1, 2-a][1,4]diazepin-5-one (7-31)

(S)-2-(4-Aminophenyl)-8-((5-(((2R,11aS)-2-fluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-5H-benzo[e]pyrrolo[1,2-a][1,4]diazepin-5-one (88 mg, 114.1 μmol)) was dissolved in dichloromethane (3 mL), followed by a dropwise addition of trifluoroacetic acid (1.5 mL) under stirring. After the addition was completed, the reaction mixture was stirred continuously for 1 hour, concentrated under reduced pressure, purified by reverse phase column chromatography (acetonitrile/1% formic acid aqueous solution=0% to 70%) and then freeze-dried to obtain the title compound (8.0 mg, 11.7 μmol).

Its structural characterization data are as follows:

ESI-MS (m/z): 673.3 [M+H]⁺

¹H NMR (400 MHz, DMSO): δ 7.39 (s, 1H), 7.29 (s, 2H), 7.14 (d, J=7.8 Hz, 2H), 6.58 (d, J=7.4 Hz, 2H), 6.29 (d, J=10.0 Hz, 2H), 5.26 (d, J=53.6 Hz, 1H), 4.21-4.08 (m, 1H), 4.01-3.89 (m, 4H), 3.85-3.76 (m, 2H), 3.65 (d, J=5.8 Hz, 6H), 3.60-3.49 (m, 4H), 3.29-3.11 (m, 4H), 3.04-2.95 (m, 1H), 2.74-2.63 (m, 1H), 1.88-1.73 (m, 4H), 1.62-1.50 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 16: Preparation of (S)-2-(4-aminophenyl)-7-methoxy-8-((5-(((2S,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-32)

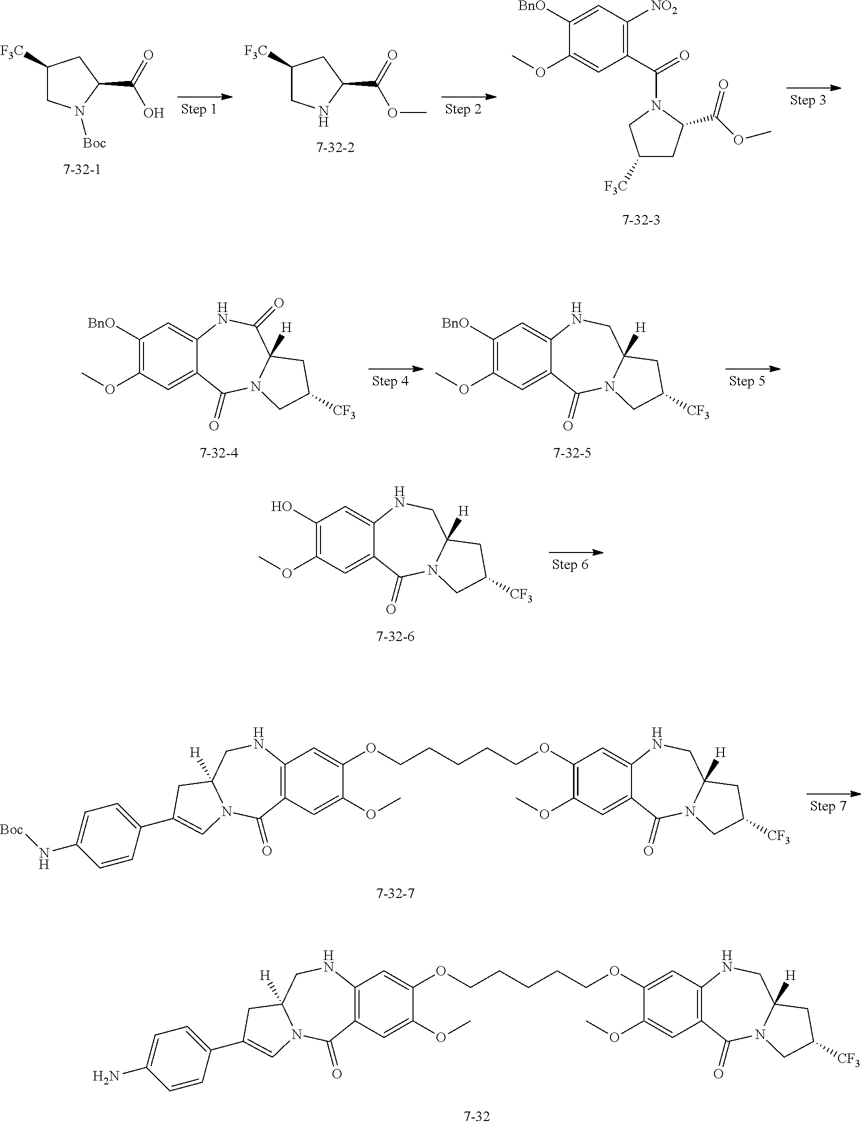

### Step 1: Preparation of methyl (2S,4S)-4-(trifluoromethyl)pyrrolidine-2-carboxylate (7-32-2)

1-(Tert-butyl)2-methyl(2S,4S)-4-(trifluoromethyl)pyrrolidine-1,2-dicarboxylic acid (1.0 g, 3.53 mmol, FR) was dissolved in methanol (10 mL), followed by an addition of HCl (4M in EA) (5 mL), and reacted at room temperature for 1 hour. The reaction mixture was concentrated to obtain a crude product (820.79 mg, 3.51 mmol), which was used directly in the next step without purification.

Its structural characterization data are as follows:

ESI-MS (m/z): 234.2 [M+H]⁺

### Step 2: Preparation of methyl (2R,4S)-1-(4-(benzyloxy)-5-methoxy-2-nitrobenzoyl)-4-(trifluoromethyl)pyrrolidin-2-carboxylate (7-32-3)

Methyl (2S,4S)-4-(trifluoromethyl)pyrrolidine-2-carboxylate (820.79 mg, 3.51 mmol, CL) and 4-(benzyloxy)-5-methoxy-2-nitrobenzoic acid (1.18 g, 3.88 mmol, FR) were dissolved in DMF (10 mL), followed by an addition of HATU (1.48 g, 3.88 mmol) and DIPEA (1.36 g, 10.59 mmol), and reacted at room temperature for 1 hour. The reaction mixture was added to ethyl acetate (100 mL) and saturated brine (100 mL), stirred, and allowed to stand for liquid separation. The organic phase was dried over anhydrous sodium sulfate, filtered and concentrated to obtain a crude product, which was purified by silica gel column (ethyl acetate/petroleum ether=20% to 60%) and concentrated again to obtain the title compound (1.5 g, 3.1 mmol).

Its structural characterization data are as follows:

ESI-MS (m/z): 483.2 [M+H]⁺

### Step 3: Preparation of (2S,11aS)-8-(benzyloxy)-7-methoxy-2-(trifluoromethyl)-1,2,3,11a-tetrahydro-5H-benzo[e]pyrrolo[1,2-a][1,4]diazepin-5,11(10H)-dione (7-32-4)

Methyl (2S,4R)-1-(4-(benzyloxy)-5-methoxy-2-nitrobenzoyl)-4-(trifluoromethyl)pyrrolidin-2-carboxylate (1.5 g, 3.1 mmol) was dissolved in methanol (30 mL), followed by an addition of zinc powder (2.2 g, 35.1 mmol) and a saturated aqueous solution of ammonium chloride (40 mL), heated to 85° C. and reacted under refluxing for 16 hours. The reaction mixture was added to ethyl acetate (150 mL) and saturated brine (100 mL), stirred, and allowed to stand for liquid separation. The organic phase was dried over anhydrous sodium sulfate and concentrated to obtain a crude product of the title compound (1.4 g, 3.3 mmol), which was used directly in the next step without purification.

Its structural characterization data are as follows:

ESI-MS (m/z): 421.2 [M+H]⁺

### Step 4: Preparation of (2S,11aS)-8-(benzyloxy)-7-methoxy-2-(trifluoromethyl)-1,2,3, 10,11,11a-hexahydro-5H-benzo[e]pyrrolo[1,2-a][1,4]diazepin-5-one (7-32-5)

(2S,11aS)-8-(Benzyloxy)-7-methoxy-2-(trifluoromethyl)-1,2,3,11a-tetrahydro-5H-benzo[e]pyrrolo[1,2-a][1,4]diazepin-5,11(10H)-dione (400 mg, 0.95 mmol) was dissolved in tetrahydrofuran (5 mL) and cooled to 0° C. Then, tetrahydrofuran borane complex (10.3 mL) was added dropwise. After the addition was completed, the reaction mixture was heated to room temperature, reacted for 3 hours, followed by a dropwise addition of methanol (10 ml), and then concentrated to obtain a crude product of the title compound, which was purified by silica gel column (ethyl acetate/petroleum ether=0% to 70%) to obtain the title compound (100 mg, 0.24 mmol).

Its structural characterization data are as follows:

ESI-MS (m/z): 408.1 [M+H]⁺

### Step 5: Preparation of (2S,11aS)-8-hydroxy-7-methoxy-2-(trifluoromethyl)-1,2,3,10,11,11a-hexahydro-5H-benzo[e]pyrrolo[1,2-a][1,4]diazepin-5-one (7-32-6)

(2S,11aS)-8-(Benzyloxy)-7-methoxy-2-(trifluoromethyl)-1,2,3,10,11,11a-hexahydro-5H-benzo[e]pyrrolo[1,2-a][1,4]diazepin-5-one (100 mg, 0.24 mmol) was dissolved in tetrahydrofuran (3 mL), followed by an addition of palladium-on-carbon (10 mg), and reacted under a hydrogen atmosphere for 1 hour. After the reaction was completed, the reaction mixture was filtered, and the filtrate was dried over anhydrous sodium sulfate, filtered and concentrated to obtain a crude product of the title compound (70 mg, 0.22 mmol), which was used directly in the next step without purification.

Its structural characterization data are as follows:

ESI-MS (m/z): 317.1 [M+H]⁺

### Step 6: Preparation of (S)-2-(4-aminophenyl)-7-methoxy-8-((5-(((2S,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-32-7)

(2R,11aS)-8-Hydroxy-7-methoxy-2-(trifluoromethyl)-1,2,3,10,11,11a-hexahydro-5H-benzo[e]pyrrolo[1,2-a][1,4]diazepin-5-one (70 mg, 0.22 mmol) and tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (136 mg, 0.23 mmol) were dissolved in DMF (2 mL), followed by an addition of potassium carbonate (61 mg, 0.44 mmol), and reacted under stirring for 16 hours. The reaction was quenched with water, and the reaction mixture was extracted with ethyl acetate for 3 times (10 mL×3). The organic phases were combined, washed with saturated brine, dried, concentrated, purified by silica gel column (MeOH/DCM=0% to 5%) and concentrated again to obtain the title compound (72 mg, 88.3 μmol).

Its structural characterization data are as follows:

ESI-MS (m/z): 822.5 [M+H]⁺

### Step 7: Preparation of (S)-2-(4-aminophenyl)-7-methoxy-8-((5-(((2S,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-32)

(S)-2-(4-aminophenyl)-7-methoxy-8-((5-(((2S,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (72 mg, 88.3 μmol) was dissolved in dichloromethane (3 mL), followed by a dropwise addition of trifluoroacetic acid (1.5 mL) under stirring. After the addition was completed, the reaction mixture was stirred continuously for 1 hour, concentrated under reduced pressure, purified by reverse phase column chromatography (acetonitrile/1% formic acid aqueous solution=0% to 70%) and then freeze-dried to obtain the title compound (9.8 mg, 13.6 μmol).

Its structural characterization data are as follows:

ESI-MS (m/z): 723.3 [M+H]⁺

¹H NMR (400 MHz, DMSO): δ 7.41 (s, 1H), 7.27 (d, J=12.2 Hz, 2H), 7.11 (d, J=8.2 Hz, 2H), 6.53 (d, J=8.4 Hz, 2H), 6.29 (d, J=12.4 Hz, 2H), 5.20 (s, 2H), 4.19-4.08 (m, 1H), 3.97-3.88 (m, 4H), 3.87-3.76 (m, 2H), 3.66 (s, 3H), 3.64 (s, 3H), 3.59-3.46 (m, 4H), 3.28-3.16 (m, 4H), 3.04-2.94 (m, 1H), 2.73-2.64 (m, 1H), 1.84-1.76 (m, 4H), 1.61-1.50 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Example 17: Preparation of (S)-2-(4-aminophenyl)-7-methoxy-8-((5-(((2R,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-33)

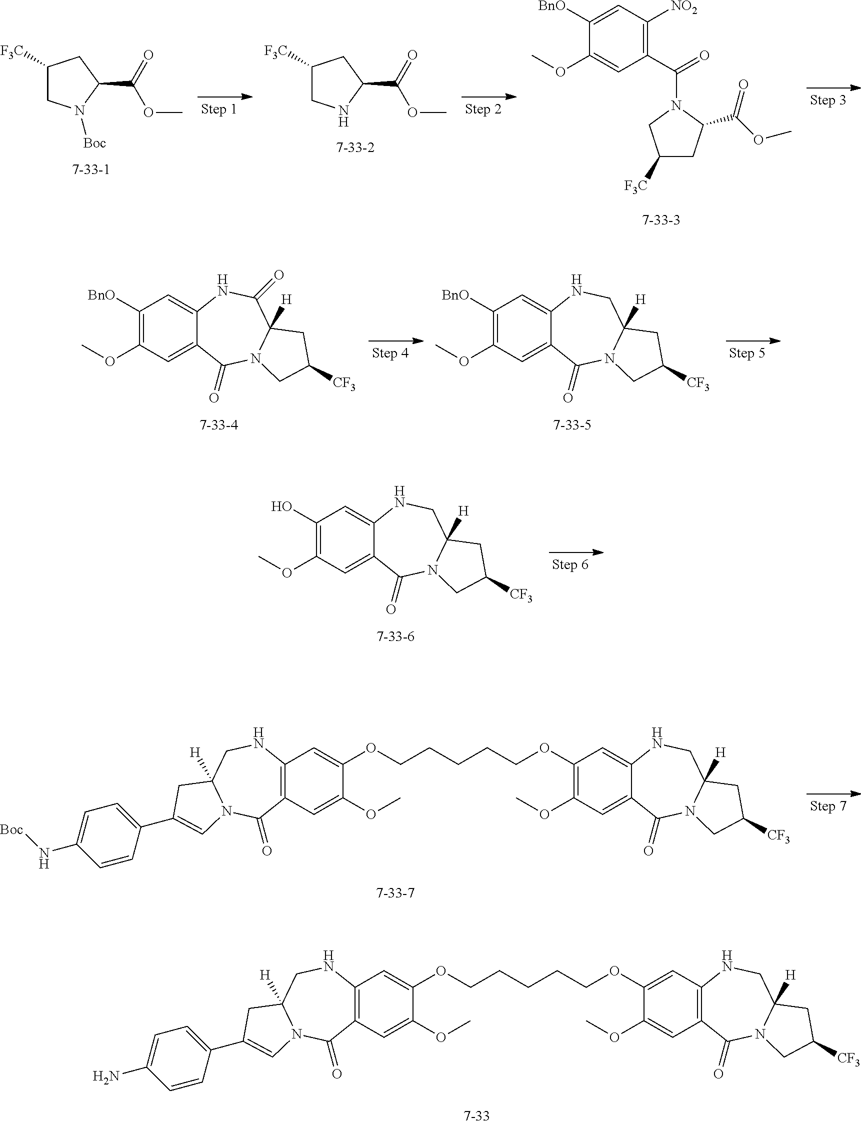

### Step 1: Preparation of methyl (2S,4R)-4-(trifluoromethyl)pyrrolidin-2-carboxylate (7-33-2)

1-(Tert-butyl)2-methyl(2S,4R)-4-(trifluoromethyl)pyrrolidine-1,2-dicarboxylic acid (1.0 g, 3.53 mmol) was dissolved in methanol (10 mL), followed by an addition of HCl (4M in EA) (5 mL), and reacted at room temperature for 1 hour. The reaction mixture was concentrated to obtain a crude product (824.79 mg, 3.53 mmol), which was used directly in the next step without purification.

Its structural characterization data are as follows:

ESI-MS (m/z): 234.2 [M+H]⁺

### Step 2: Preparation of methyl (2S,4R)-1-(4-(benzyloxy)-5-methoxy-2-nitrobenzoyl)-4-(trifluoromethyl)pyrrolidin-2-carboxylate (7-33-3)

Methyl (2S,4R)-4-(trifluoromethyl)pyrrolidin-2-carboxylate (824.79 mg, 3.53 mmol) and 4-(benzyloxy)-5-methoxy-2-nitrobenzoic acid (1.18 g, 3.88 mmol) were dissolved in DMF (10 mL), followed by an addition of HATU (1.48 g, 3.88 mmol) and DIPEA (1.36 g, 10.59 mmol), and reacted at room temperature for 1 hour. The reaction mixture was added to ethyl acetate (100 mL) and saturated brine (100 mL), stirred, and allowed to stand for liquid separation. The organic phase was dried over anhydrous sodium sulfate, filtered and concentrated to obtain a crude product, which was purified by silica gel column (ethyl acetate/petroleum ether=20% to 60%) and concentrated again to obtain the title compound (1.7 g, 3.52 mmol).

Its structural characterization data are as follows:

ESI-MS (m/z): 483.2 [M+H]⁺

### Step 3: Preparation of (2R,11aS)-8-(benzyloxy)-7-methoxy-2-(trifluoromethyl)-1,2,3,11a-tetrahydro-5H-benzo[e]pyrrolo[1,2-a][1,4]diazepin-5,11(10H)-dione (7-33-4)

Methyl (2S,4R)-1-(4-(benzyloxy)-5-methoxy-2-nitrobenzoyl)-4-(trifluoromethyl)pyrrolidin-2-carboxylate (1.7 g, 3.52 mmol) was dissolved in methanol (30 mL), followed by an addition of zinc powder (2.29 g, 35.24 mmol) and a saturated aqueous solution of ammonium chloride (40 mL), heated to 85° C. and reacted under refluxing for 16 hours. The reaction mixture was added to ethyl acetate (150 mL) and saturated brine (100 mL), stirred, and allowed to stand for liquid separation. The organic phase was dried over anhydrous sodium sulfate and concentrated to obtain a crude product of the title compound (1.48 g, 3.52 mmol), which was used directly in the next step without purification.

Its structural characterization data are as follows:

ESI-MS (m/z): 421.2 [M+H]⁺

### Step 4: Preparation of (2R,11aS)-8-(benzyloxy)-7-methoxy-2-(trifluoromethyl)-1,2,3,10,11,11a-hexahydro-5H-benzo[e]pyrrolo[1,2-a][1,4]diazepin-5-one (7-33-5)

(2R,11aS)-8-(Benzyloxy)-7-methoxy-2-(trifluoromethyl)-1,2,3,11a-tetrahydro-5H-benzo[e]pyrrolo[1,2-a][1,4]diazepin-5,11(10H)-dione (1.48 g, 3.52 mmol) was dissolved in tetrahydrofuran (30 mL), followed by an addition of sodium borohydride (770.88 mg, 10.56 mmol) and a dropwise addition of trifluoroacetic acid (802.56 mg, 7.04 mmol) at room temperature until no large amount of gas was released, heated to 75° C. and reacted under refluxing for 5 hours. The reaction mixture was concentrated, followed by an addition of water (50 mL), and then extracted twice with ethyl acetate (50 mL×2). The organic phases were combined, washed with saturated sodium chloride aqueous solution, dried over anhydrous sodium sulfate, filtered and concentrated to obtain a crude product of the title compound (1.1 g, 2.71 mmol), which was used directly in the next step without purification.

Its structural characterization data are as follows:

ESI-MS (m/z): 408.1 [M+H]⁺

### Step 5: Preparation of (2R,11aS)-8-hydroxy-7-methoxy-2-(trifluoromethyl)-1,2,3,10,11,11a-hexahydro-5H-benzo[e]pyrrolo[1,2-a][1,4]diazepin-5-one (7-33-6)

(2R,11aS)-8-(Benzyloxy)-7-methoxy-2-(trifluoromethyl)-1,2,3,10,11,11a-hexahydro-5H-benzo[e]pyrrolo[1,2-a][1,4]diazepin-5-one (100 mg, 246.06 μmol) was dissolved in methanol (10 mL), followed by an addition of palladium-on-carbon (5 mg), and reacted under a hydrogen atmosphere for 1 hour. After the reaction was completed, the reaction mixture was filtered, and the filtrate was dried over anhydrous sodium sulfate, filtered and concentrated to obtain a crude product of the title compound (77.82 mg, 246.05 μmol), which was used directly in the next step without purification.

Its structural characterization data are as follows:

ESI-MS (m/z): 317.1 [M+H]⁺

### Step 6: Preparation of (S)-2-(4-aminophenyl)-7-methoxy-8-((5-(((2R,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-33-7)

(2R,11aS)-8-Hydroxy-7-methoxy-2-(trifluoromethyl)-1,2,3,10,11,11a-hexahydro-5H-benzo[e]pyrrolo[1,2-a][1,4]diazepin-5-one (10.78 mg, 34.10 μmol) and tert-butyl (S)-(4-(8-((5-bromopentyl)oxy)-7-methoxy-5-oxo-5,10,11,11a-tetrahydro-1H-benzo[e]pyrrolo[1,2-a][1,4]diazepin-2-yl)phenyl)carbamate (20.00 mg, 34.10 μmol) were dissolved in DMF (1 mL), followed by an addition of potassium carbonate (14.12 mg, 102.30 μmol), and reacted under stirring for 16 hours. The reaction was quenched with water, and the reaction mixture was extracted with ethyl acetate for 3 times (10 mL×3). The organic phases were combined, washed with saturated brine, dried, concentrated, purified by silica gel column (MeOH/DCM=0% to 5%) and concentrated again to obtain the title compound (20 mg, 24.33 μmol).

Its structural characterization data are as follows:

ESI-MS (m/z): 822.5 [M+H]⁺

### Step 7: Preparation of (S)-2-(4-aminophenyl)-7-methoxy-8-((5-(((2R,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (7-33)

(S)-2-(4-Aminophenyl)-7-methoxy-8-((5-(((2R,11aS)-7-methoxy-5-oxo-2-(trifluoromethyl)-2,3,5,10,11,11a-hexahydro-1H-benzo[e]pyrrolo[1,2-a][1,4]diazepin-8-yl)oxy)pentyl)oxy)-1,10,11,11a-tetrahydro-5H-benzo[e]pyrrolo[1,2-a][1,4]diazepin-5-one (20 mg, 24.33 μmol) was dissolved in dichloromethane (2 mL), followed by a dropwise addition of trifluoroacetic acid (0.5 mL) under stirring. After the addition was completed, the reaction mixture was stirred continuously for 1 hour, concentrated under reduced pressure, purified by reverse phase column chromatography (acetonitrile/1% formic acid aqueous solution=0% to 70%) and then freeze-dried to obtain the title compound (9.33 mg, 12.80 μmol).

Its structural characterization data are as follows:

ESI-MS (m/z): 723.3 [M+H]⁺

¹H NMR (400 MHz, DMSO) δ 7.39 (s, 1H), 7.28 (s, 1H), 7.25 (s, 1H), 6.82 (s, 2H), 6.59-6.43 (m, 2H), 6.31 (s, 2H), 4.18-4.08 (m, 1H), 3.97-3.85 (m, 4H), 3.82-3.74 (m, 2H), 3.68 (s, 3H), 3.66 (s, 3H), 3.56-3.44 (m, 4H), 3.28-3.19 (m, 2H), 2.81-2.65 (m, 2H), 2.21-2.04 (m, 2H), 1.87-1.70 (m, 4H), 1.59-1.52 (m, 2H).

Its preparation method was as follows:
Chromatographic column: SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 90 | 28 |
| 2.00 | 30 | 90 | 28 |
| 20.00 | 90 | 10 | 28 |

### Preparation Examples 18 and 19: Preparation of (S)-8-((5-(((2S,11aS)-2-(4-aminophenyl)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-34) and (S)-8-((5-(((2R,11aS)-2-(4-aminophenyl)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-1,10,11,11a-tetrahydro-3H,5H-spiro[e]pyrrolo[1,2-a][1,4]azepine-2,1′-cyclopropane]-5-one (7-35)

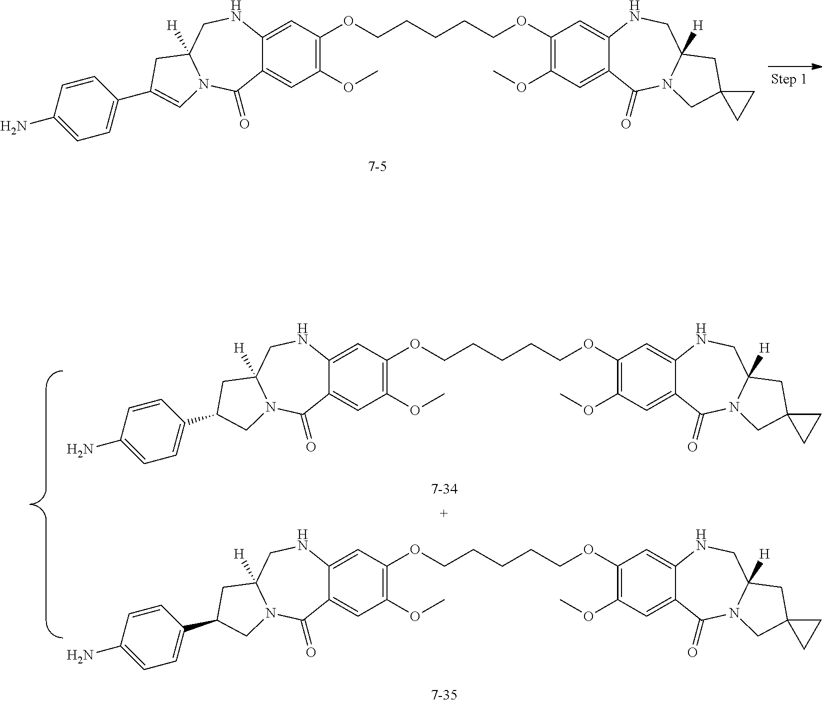 [SMILES: COc1cc2c(cc1OCCCCCOc1cc3c(cc1OC)C(=O)N1CC4(CC4)C[C@H]1CN3)NC[C@@H]1CC(c3ccc(N)cc3)=CN1C2=O ; COc1cc2c(cc1OCCCCCOc1cc3c(cc1OC)C(=O)N1CC4(CC4)C[C@H]1CN3)NC[C@@H]1C[C@@H](c3ccc(N)cc3)CN1C2=O ; COc1cc2c(cc1OCCCCCOc1cc3c(cc1OC)C(=O)N1CC4(CC4)C[C@H]1CN3)NC[C@@H]1C[C@H](c3ccc(N)cc3)CN1C2=O]

(S)-8-((5-(((S)-2-(4-Aminophenyl)-7-methoxy-5-oxo-5,10,11,11a-tetrahydro-1H-benzo[e]pyrrolo[1,2-a][1,4]diazepin-8-yl)oxy)pentyl)oxy)-7-ethoxy-1,10,11,11-tetrahydro-3H,5H-spiro[e]pyrrolo[1,2-a][1,4]azepine-2,1′-cyclopropane]-5-one (Compound 7-5) (20 mg, 29.42 μmol) was dissolved in a mixed solvent of methanol (5 mL) and tetrahydrofuran (5 mL), followed by an addition of 10% palladium-on-carbon (2 mg), subjected to hydrogen replacement, and stirred under stirring and protection for 2 hours. The reaction mixture was filtered through diatomaceous earth, and the filtrate was concentrated under reduced pressure, purified by preparative high performance liquid chromatography, and freeze-dried to obtain Compound 7-34 (2.77 mg, 3.13 μmol) and Compound 7-35 (5.66 mg, 6.69 μmol).

Their structural characterization data are as follows:

MS m/z (ESI): double peak 682.5 [M+H]⁺

Compound 7-34: ¹H NMR (400 MHz, DMSO) δ 7.40-7.24 (m, 4H), 7.10 (d, J=8.3 Hz, 2H), 6.31 (t, J=13.0 Hz, 2H), 3.97 (s, 1H), 3.92 (t, J=6.2 Hz, 4H), 3.83 (d, J=7.6 Hz, 2H), 3.65 (d, J=5.1 Hz, 6H), 3.55-3.47 (m, 4H), 3.30 (dd, J=19.7, 10.5 Hz, 2H), 3.26-3.11 (m, 2H), 2.31 (dd, J=12.5, 7.5 Hz, 1H), 2.17-2.09 (m, 1H), 1.96 (dd, J=12.4, 7.2 Hz, 1H), 1.86-1.72 (m, 5H), 1.60-1.51 (m, 2H), 0.68-0.48 (m, 4H).

Compound 7-35: ¹H NMR (400 MHz, DMSO) δ 7.35 (dd, J=18.7, 10.3 Hz, 4H), 7.11 (d, J=8.2 Hz, 2H), 6.29 (d, J=15.3 Hz, 2H), 4.05 (d, J=3.6 Hz, 1H), 3.92 (t, J=6.4 Hz, 4H), 3.83 (s, 2H), 3.65 (t, J=2.7 Hz, 6H), 3.53 (d, J=11.8 Hz, 4H), 3.33 (d, J=11.7 Hz, 2H), 3.12 (ddd, J=34.2, 13.2, 8.2 Hz, 2H), 2.46 (d, J=5.1 Hz, 1H), 2.00-1.93 (m, 1H), 1.87-1.70 (m, 6H), 1.56 (d, J=6.6 Hz, 2H), 0.58 (ddt, J=15.7, 12.7, 6.9 Hz, 4H).

Their preparation method was as follows:
Chromatographic column: Waters SunFire Prep C18 OBD (5 μm*19 mm*150 mm)
Mobile phase A: acetonitrile; Mobile phase B: water (0.05% trifluoroacetic acid)

| Time [min] | Mobile phase A [%] | Mobile phase B [%] | Flow rate [mL/min] |
|---|---|---|---|
| 0.00 | 10 | 90 | 28 |
| 2.00 | 10 | 90 | 28 |
| 22.00 | 90 | 10 | 28 |

### Preparation Example 20: Preparation of (S)-8-((5-(((S)-2-(furan-3-yl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-36)

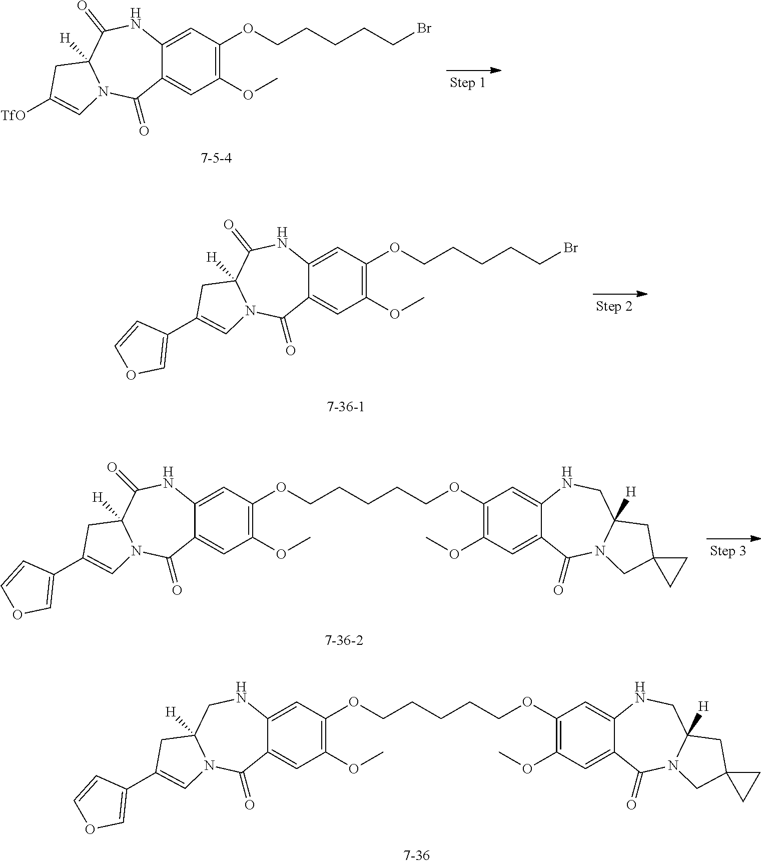 [SMILES: COc1cc2c(cc1OCCCCCBr)NC(=O)[C@@H]1CC(OS(=O)(=O)C(F)(F)F)=CN1C2=O ; COc1cc2c(cc1OCCCCCBr)NC(=O)[C@@H]1CC(c3ccoc3)=CN1C2=O ; COc1cc2c(cc1OCCCCCOc1cc3c(cc1OC)C(=O)N1CC4(CC4)C[C@H]1CN3)NC(=O)[C@@H]1CC(c3ccoc3)=CN1C2=O ; COc1cc2c(cc1OCCCCCOc1cc3c(cc1OC)C(=O)N1CC4(CC4)C[C@H]1CN3)NC[C@@H]1CC(c3ccoc3)=CN1C2=O]

### Step 1: Preparation of (S)-8-((5-bromopentyl)oxy)-2-(furan-3-yl)-7-methoxy-1,11a-dihydro-5H-benzo[e]pyrrolo[1,2-a][1,4]diazepin-5,11(10H)-dione (7-36-1)

Furan-3-ylboronic acid (40.15 mg, 358.85 μmol), (S)-8-((5-bromopentyl)oxy)-7-methoxy-5,11-dioxo-5,10,11,11a-tetrahydro-1H-benzo[e]pyrrolo[1,2-a][1,4]diazepin-2-yl trifluoromethanesulfonate (100 mg, 179.43 μmol), tetrakis(triphenylphosphine)palladium (20.73 mg, 17.94 μmol) and sodium carbonate (76.07 mg, 717.70 μmol) were mixed together, followed by an addition of water (0.5 mL), toluene (2 mL) and ethanol (0.5 mL), then subjected to nitrogen replacement for three times, heated to 80° C. and reacted for 3 hours. After the reaction was completed, 10 mL of water was added to the reaction solution, and extracted with ethyl acetate (5 mL×3). The organic phases were combined and washed with 10 mL of saturated brine, dried over anhydrous sodium sulfate, and concentrated under reduced pressure to obtain a crude product, which was purified by silica gel column chromatography (ethyl acetate/petroleum ether=0% to 70%) and concentrated under reduced pressure again to obtain the title compound (60 mg, 126.23 μmol).

Its structural characterization data are as follows:

ESI-MS (m/z): 475.1 [M+H]⁺

### Step 2: Preparation of (S)-2-(furan-3-yl)-7-methoxy-8-((5-(((S)-7-methoxy-5-oxo-5,10,11, 11a-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (7-36-2)

(S)-8-((5-Bromopentyl)oxy)-2-(furan-3-yl)-7-methoxy-1,11a-dihydro-5H-benzo[e]pyrrolo[1,2-a][1,4]diazepin-5,11(10H)-dione (60 mg, 126.23 μmol) and (S)-8-hydroxy-7-methoxy-1,10,11,11a-tetrahydro-3H,5H-spiro[benzo[e]pyrrolo[1,2-a][1,4]diazepin-2,1′-cyclopropane]-5-one (34.63 mg, 126.23 μmol) were dissolved in DMF (1 mL), followed by an addition of potassium carbonate (52.34 mg, 378.68 μmol), and reacted under stirring at room temperature for 20 hours. After the reaction was completed, the reaction mixture was treated with 10 mL of water to precipitate a large amount of solid, and extracted with 10 mL of a mixed solvent of ethyl acetate:methanol=10:1 for 3 times. The organic phases were combined and washed with 10 mL of brine, dried over anhydrous sodium sulfate, and concentrated under reduced pressure to obtain a crude product, which was purified by preparative thin layer chromatography (DCM:MeOH=8:1) and concentrated again under reduced pressure to obtain the title compound (43 mg, 50.80 μmol).

Its structural characterization data are as follows:

ESI-MS (m/z): 669.3 [M+H]⁺

### Step 3: Preparation of (S)-8-((5-(((S)-2-(furan-3-yl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-36)

(S)-2-(Furan-3-yl)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (24 mg, 35.89 μmol) was dissolved in THF (2 mL), followed by an addition of NaBH₄ (12.22 mg, 323.00 μmol) and a dropwise addition of TFA (24.55 mg, 215.33 μmol) to generate a large amount of bubbles in the system. After the bubbles disappeared, the reaction system was transferred to an oil bath, heated to 75° C. and reacted for 22 hours. After the reaction was completed, 5 mL of water was added to the reaction solution, and extracted three times with ethyl acetate (5 mL×3). The organic phases were combined, washed with 5 mL of saturated brine, dried over anhydrous sodium sulfate, and concentrated under reduced pressure to obtain a crude product, which was purified by preparative HPLC and freeze-dried to obtain the title compound (6.56 mg, 9.77 μmol).

Its structural characterization data are as follows:

ESI-MS (m/z): 655.4 [M+H]⁺

¹H NMR (400 MHz, DMSO-d6) δ 7.66 (d, J=10.0 Hz, 2H), 7.32 (s, 2H), 7.28 (s, 1H), 6.85 (s, 1H), 6.53 (d, J=6.4 Hz, 1H), 6.31-6.26 (m, 3H), 4.20-4.13 (m, 1H), 3.98-3.88 (m, 4H), 3.88-3.70 (m, 1H), 3.66 (s, 3H), 3.64 (s, 3H), 3.58-3.43 (m, 4H), 3.25-3.11 (m, 4H), 2.62 (dd, J=16.8, 4.4 Hz, 1H), 1.95 (dd, J=12.4, 7.2 Hz, 1H), 1.79 (dd, J=12.4, 7.2 Hz, 5H), 1.61-1.49 (m, 2H), 0.68-0.61 (m, 1H), 0.60-0.48 (m, 3H).

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 32 |
| 4.00 | 30 | 70 | 32 |
| 24.00 | 90 | 10 | 32 |

### Preparation Example 21: Preparation of (S)-7-methoxy-8-((5-(((S)-7-methoxy-5-oxo-2-(thien-3-yl)-5,10,11,11a-tetrahydro-1H-benzo[e]pyrrolo[1,2-a][1,4]diazepin-8-yl)oxy)pentyl)oxy)-1,10,11,11a-tetrahydro-3H,5H-spiro[benzo[e]pyrrolo[1,2-a][1,4]diazepin-2,1′-cyclopropane]-5-one (7-37)

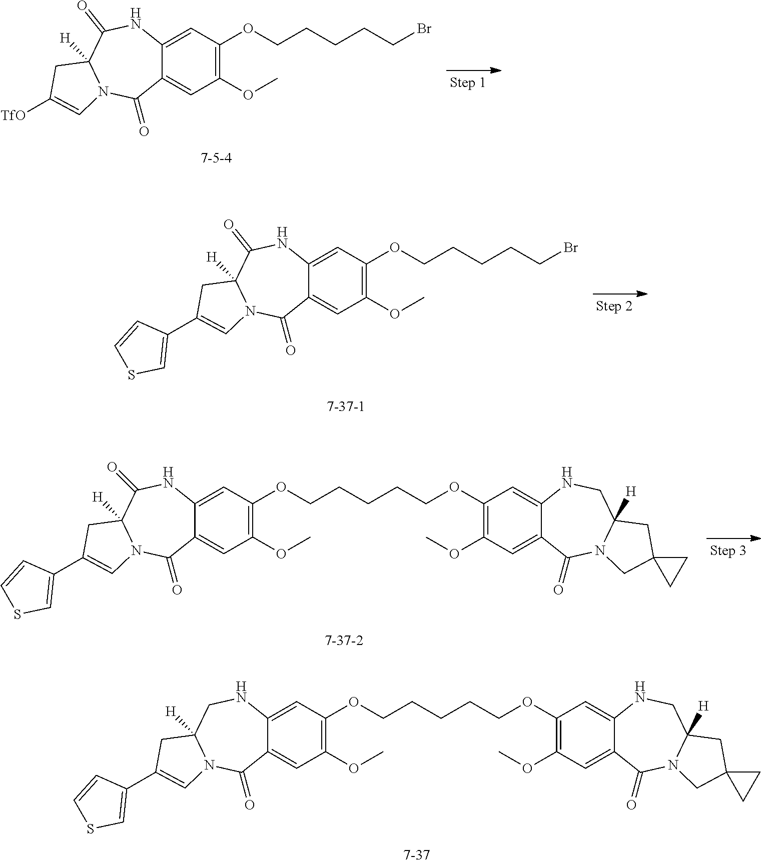 [SMILES: COc1cc2c(cc1OCCCCCBr)NC(=O)[C@@H]1CC(OS(=O)(=O)C(F)(F)F)=CN1C2=O ; COc1cc2c(cc1OCCCCCBr)NC(=O)[C@@H]1CC(c3ccsc3)=CN1C2=O ; COc1cc2c(cc1OCCCCCOc1cc3c(cc1OC)C(=O)N1CC4(CC4)C[C@H]1CN3)NC(=O)[C@@H]1CC(c3ccsc3)=CN1C2=O ; COc1cc2c(cc1OCCCCCOc1cc3c(cc1OC)C(=O)N1CC4(CC4)C[C@H]1CN3)NC[C@@H]1CC(c3ccsc3)=CN1C2=O]

### Step 1: Preparation of (S)-8-((5-bromopentyl)oxy)-2-(furan-3-yl)-7-methoxy-1,11a-dihydro-5H-benzo[e]pyrrolo[1,2-a][1,4]diazepin-5,11(10H)-dione (7-37-1)

Thiophene-3-ylboronic acid (91.84 mg, 717.70 μmol), (S)-8-((5-bromopentyl)oxy)-7-methoxy-5,11-dioxo-5,10,11,11a-tetrahydro-1H-benzo[e]pyrrolo[1,2-a][1,4]diazepin-2-yl trifluoromethanesulfonate (200 mg, 358.85 μmol), tetrakis(triphenylphosphine)palladium (41.47 mg, 35.89 μmol) and sodium carbonate (152.14 mg, 1.44 mmol) were mixed together, followed by an addition of water (1 mL), toluene (4 mL) and ethanol (1 mL), subjected to nitrogen replacement for three times, heated to 80° C. and reacted for 2 hours. After the reaction was completed, 10 mL of water was added to the reaction solution, and extracted three times with ethyl acetate (5 mL×3). The organic phases were combined, washed with 10 mL of saturated brine, dried over anhydrous sodium sulfate, and concentrated under reduced pressure to obtain a crude product, which was purified by silica gel column chromatography (ethyl acetate/petroleum ether=0% to 70%) and concentrated under reduced pressure again to obtain the title compound (58 mg, 118.03 μmol).

Its structural characterization data are as follows:

ESI-MS (m/z): 491.1 [M+H]⁺

### Step 2: Preparation of (S)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropyl]-8-yl)oxy)pentyl)oxy)-2-(thien-3-yl)-1,11a-dihydro-5H-benzo[e]pyrrolo[1,2-a][1,4]diazepin-5,11(10H)-dione (7-37-2)

(S)-8-((5-Bromopentyl)oxy)-2-(furan-3-yl)-7-methoxy-1,11a-dihydro-5H-benzo[e]pyrrolo[1,2-a][1,4]diazepin-5,11(10H)-dione (55 mg, 111.93 μmol) and (S)-8-hydroxy-7-methoxy-1,10, 11,11a-tetrahydro-3H,5H-spiro[benzo[e]pyrrolo[1,2-a][1,4]diazepin-2,1′-cyclopropane]-5-one (30.70 mg, 111.93 μmol) were dissolved in DMF (2 mL), followed by an addition of potassium carbonate (46.41 mg, 335.78 μmol), and reacted under stirring at room temperature for 40 hours. After the reaction was completed, the reaction mixture was filtered to remove the insoluble matter, and the filtrate was purified by preparative high performance liquid chromatography and freeze-dried to obtain the title compound (15 mg, 21.90 μmol).

Its structural characterization data are as follows:

ESI-MS (m/z): 685.3 [M+H]⁺

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 32 |
| 4.00 | 30 | 70 | 32 |
| 24.00 | 90 | 10 | 32 |

### Step 3: Preparation of (S)-7-methoxy-8-((5-(((S)-7-methoxy-5-oxo-2-(thien-3-yl)-5,10,11,11a-tetrahydro-1H-benzo[e]pyrrolo[1,2-a][1,4]diazepin-8-yl)oxy)pentyl)oxy)-1,10,11,11a-tetrahydro-3H,5H-spiro[benzo[e]pyrrolo[1,2-a][1,4]diazepin-2,1′-cyclopropane]-5-one (7-37)

(S)-7-Methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropyl]-8-yl)oxy)pentyl)oxy)-2-(thien-3-yl)-1,11a-dihydro-5H-benzo[e]pyrrolo[1,2-a][1,4]diazepin-5,11(10H)-dione (15 mg, 21.90 μmol) was dissolved in THF (2 mL), followed by an addition of sodium borohydride (8.29 mg, 219.04 μmol) to generate a small amount of bubbles and a dropwise addition with TFA (14.99 mg, 131.43 μmol) to generate a large amount of bubbles, and then heated to 70° C. and reacted for 24 hours. After the reaction was completed, the reaction mixture was concentrated under reduced pressure, further purified by preparative HPLC and freeze-dried to obtain the title compound (2.83 mg, 4.01 μmol).

Its structural characterization data are as follows:

ESI-MS (m/z): 671.3 [M+H]⁺

¹H NMR (400 MHz, DMSO-d6) δ 7.53 (dd, J=4.8, 2.8 Hz, 1H), 7.45 (dd, J=3.6, 1.2 Hz, 2H), 7.32 (s, 1H), 7.29-7.28 (m, 2H), 6.55 (d, J=6.0 Hz, 1H), 6.31 (s, 1H), 6.30-6.26 (m, 2H), 4.21-4.15 (m, 1H), 3.93 (dd, J=13.2, 6.6 Hz, 4H), 3.83 (dd, J=15.2, 7.6 Hz, 1H), 3.66 (s, 3H), 3.64 (s, 3H), 3.55-3.50 (m, 2H), 3.50-3.44 (m, 1H), 3.24 (dd, J=13.2, 10.0 Hz, 2H), 3.14 (dd, J=12.0, 8.8 Hz, 1H), 2.74 (dd, J=16.4, 4.0 Hz, 1H), 2.03-1.90 (m, 2H), 1.79 (dd, J=12.4, 7.6 Hz, 5H), 1.61-1.50 (m, 2H), 0.68-0.61 (m, 1H), 0.61-0.48 (m, 3H).

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 28 |
| 4.00 | 30 | 70 | 28 |
| 24.00 | 90 | 10 | 28 |

### Preparation Example 22: Preparation of (S)-8-((5-(((S)-2-(5-(hydroxymethyl)thien-3-yl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-38)

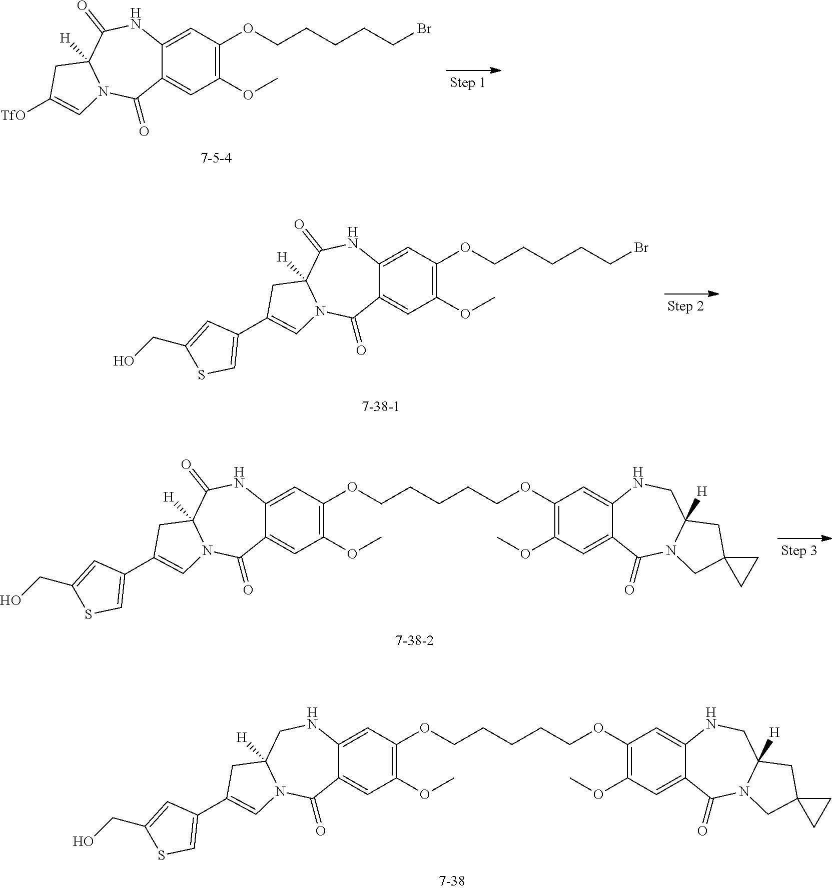 [SMILES: COc1cc2c(cc1OCCCCCBr)NC(=O)[C@@H]1CC(OS(=O)(=O)C(F)(F)F)=CN1C2=O ; COc1cc2c(cc1OCCCCCBr)NC(=O)[C@@H]1CC(c3csc(CO)c3)=CN1C2=O ; COc1cc2c(cc1OCCCCCOc1cc3c(cc1OC)C(=O)N1CC4(CC4)C[C@H]1CN3)NC(=O)[C@@H]1CC(c3csc(CO)c3)=CN1C2=O ; COc1cc2c(cc1OCCCCCOc1cc3c(cc1OC)C(=O)N1CC4(CC4)C[C@H]1CN3)NC[C@@H]1CC(c3csc(CO)c3)=CN1C2=O]

### Step 1: Preparation of (S)-8-((5-bromopentyl)oxy)-2-(5-(hydroxymethyl)thien-3-yl)-7-methoxy-1,11a-dihydro-5H-benzo[e]pyrrolo[1,2-a][1,4]diazepin-5,11(10H)-dione (7-38-1)

(5-(Hydroxymethyl)thien-3-yl)boronic acid (113.38 mg, 717.70 μmol), (S)-8-((5-bromopentyl)oxy)-7-methoxy-5,11-dioxo-5,10,11,11a-tetrahydro-1H-benzo[e]pyrrolo[1,2-a][1,4]diaza-2-yl trifluoromethanesulfonate (200 mg, 358.85 μmol), tetrakis(triphenylphosphine)palladium (41.47 mg, 35.89 μmol) and sodium carbonate (152.14 mg, 1.44 mmol) were mixed together, followed by an addition of water (1 mL), toluene (4 mL) and ethanol (1 mL), then subjected to nitrogen replacement for three times, heated to 80° C. and reacted for 2 hours. After the reaction was completed, 10 mL of water was added to the reaction solution, and extracted three times with ethyl acetate (5 mL×3). The organic phases were combined, washed with 10 mL of saturated brine, dried over anhydrous sodium sulfate, and concentrated under reduced pressure to obtain a crude product, which was purified by silica gel column chromatography (ethyl acetate/petroleum ether=0% to 100%) and then concentrated under reduced pressure to obtain the title compound (30 mg, 50.06 μmol).

Its structural characterization data are as follows:

ESI-MS (m/z): 521.1 [M+H]⁺

### Step 2: Preparation of (S)-2-(5-(hydroxymethyl)thiophene-3-yl)-7-methoxy-8-((5-(((S)-7-methoxy-5-oxo-5,10,11,11a-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (7-38-2)

(S)-8-((5-Bromopentyl)oxy)-2-(5-(hydroxymethyl)thien-3-yl)-7-methoxy-1,11a-dihydro-5H-benzo[e]pyrrolo[1,2-a][1,4]diazepin-5,11(10H)-dione (30 mg, 57.53 μmol) and (S)-8-hydroxy-7-methoxy-1,10,11,11a-tetrahydro-3H,5H-spiro[benzo[e]pyrrolo[1,2-a][1,4]diazepin-2,1′-cyclopropane]-5-one (15.78 mg, 57.53 μmol) were dissolved in DMF (1 mL), followed by an addition of potassium carbonate (23.85 mg, 172.60 μmol), and reacted under stirring at room temperature for 40 hours. After the reaction was completed, the reaction mixture was filtered to remove the insoluble matter, and the filtrate was purified by preparative high performance liquid chromatography and freeze-dried to obtain the title compound (10 mg, 13.99 μmol).

Its structural characterization data are as follows:

ESI-MS (m/z): 715.3 [M+H]⁺

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 32 |
| 4.00 | 30 | 70 | 32 |
| 24.00 | 90 | 10 | 32 |

### Step 3: Preparation of (S)-8-((5-(((S)-2-(5-(hydroxymethyl)thien-3-yl)-7-methoxy-5-oxo-5,10,11,11a-tetrahydro-1H-benzo[e]pyrrolo[1,2-a][1,4]diazepin-8-yl)oxy)pentyl)oxy)-7-methoxy-1,10,11,11a-tetrahydro-3H,5H-spiro[benzo[e]pyrrolo[1,2-a][1,4]diazepin-2,1′-cyclopropane]-5-one (7-38)

(S)-2-(5-(Hydroxymethyl)thien-3-yl)-7-methoxy-8-((5-(((S)-7-methoxy-5-oxo-5,10,11,11a)-tetrahydro-1H,3H-spiro[benzo[e]pyrrolo[1,2-a][1,4]diazepin-2,1′-cyclopropane]-8-yl)oxy)pentyl)oxy)-1,11a-dihydro-5H-benzo[e]pyrrolo[1,2-a][1,4]diazepin-5,11(10H)-dione (10 mg, 13.99 μmol) was dissolved in THF (2 mL), followed by an addition of sodium borohydride (5.15 mg, 139.89 μmol) to generate a small amount of bubbles and a dropwise addition of TFA (9.57 mg, 83.94 μmol), heated to 70° C., and reacted for 24 hours. After the reaction was completed, the reaction mixture was concentrated under reduced pressure, and the residue was dissolved in methanol, purified by preparative high performance liquid chromatography and freeze-dried to obtain the title compound (2.18 mg, 2.96 μmol).

Its structural characterization data are as follows:

ESI-MS (m/z): 701.3 [M+H]⁺

¹H NMR (400 MHz, DMSO-d6) δ 7.35 (s, 1H), 7.32 (s, 1H), 7.28 (s, 1H), 7.24 (s, 1H), 7.15 (d, J=1.2 Hz, 1H), 6.55 (d, J=6.4 Hz, 1H), 6.30 (d, J=10.8 Hz, 3H), 5.48 (s, 1H), 4.60 (d, J=3.6 Hz, 2H), 4.17 (dd, J=15.2, 10.0 Hz, 1H), 3.93 (dd, J=12.8, 6.4 Hz, 4H), 3.83 (dd, J=14.8, 7.2 Hz, 1H), 3.65 (d, J=8.4 Hz, 6H), 3.55-3.46 (m, 4H), 3.29-3.21 (m, 2H), 3.21-3.09 (m, 2H), 2.71 (dd, J=16.8, 4.0 Hz, 1H), 2.03-1.91 (m, 2H), 1.81-1.75 (m, 5H), 1.59-1.51 (m, 2H), 0.68-0.61 (m, 1H), 0.60-0.48 (m, 3H).

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 28 |
| 4.00 | 30 | 70 | 28 |
| 24.00 | 90 | 10 | 28 |

### Preparation Example 23: Preparation of (S)-8-((5-(((S)-2,2-difluoro-7-methoxy-5-oxo-2,3,5, 10,11,11a-hexahydro-1H-benzo[e]pyrrolo[1,2-a][1,4]diazepin-8-yl)oxy)pentyl)oxy)-7-methoxy-2-(4-methoxyphenyl)-1,10,11,11a-tetrahydro-5H-benzo[e]pyrrolo[1,2-a][1,4]diazepin-5-one (7-39)

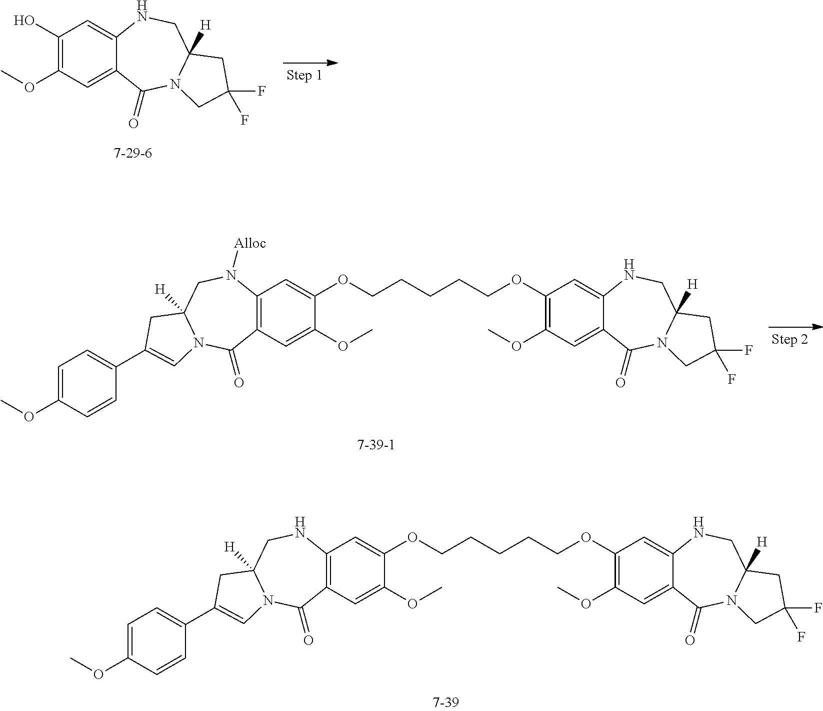

### Step 1: Preparation of allyl (S)-8-((5-(((S)-2,2-difluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (7-39-1)

(S)-2,2-Difluoro-8-hydroxy-7-methoxy-1,2,3,10,11,11a-hexahydro-5H-benzo[e]pyrrolo[1,2-a][1,4]diazepin-5-one (19 mg, 66.84 μmol) and allyl (S)-8-((5-bromopentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (39.13 mg, 66.84 μmol) were dissolved in DMF (0.5 mL), followed by an addition of potassium carbonate (27.71 mg, 200.52 μmol), and stirred at room temperature for 24 hours. Then potassium iodide (11.10 mg, 66.84 μmol) was added to the reaction mixture, which was then stirred continuously at room temperature for 48 hours. After the reaction was completed, 5 mL of water was added to the reaction solution, and extracted with ethyl acetate (5 mL×3). The organic phases were combined, washed with 5 mL of saturated brine, dried over anhydrous sodium sulfate, and concentrated under reduced pressure to obtain a crude product of the title compound (70 mg, 64.51 μmol), which was used directly in the next step without purification.

Its structural characterization data are as follows:

ESI-MS (m/z): 790.4 [M+H]⁺

### Step 2: Preparation of (S)-8-((5-(((S)-2,2-difluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-1,10,11,11a-tetrahydro-5H-benzo[e]pyrrolo[1,2-a][1,4]diazepin-5-one (7-39)

Allyl (S)-8-((5-(((S)-2,2-difluoro-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (70 mg, 88.74 μmol) was dissolved in DMF (1 mL), followed by an addition of tetrahydropyrrole (12.62 mg, 177.48 μmol) and tetrakis(triphenylphosphine)palladium (10.25 mg, 8.87 μmol), subjected to nitrogen replacement for three times, and reacted under stirring and nitrogen protection at room temperature for 3 hours. After the reaction was completed, the reaction mixture was filtered, and the filtrate was purified by preparative high performance liquid chromatography and freeze-dried to obtain the title compound (9.01 mg, 12.66 μmol).

Its structural characterization data are as follows:

ESI-MS (m/z): 705.3 [M+H]⁺

¹H NMR (400 MHz, DMSO-d6) δ 7.45 (s, 1H), 7.39 (s, 1H), 7.37 (s, 1H), 7.34 (s, 1H), 7.29 (s, 1H), 6.91 (d, J=8.8 Hz, 2H), 6.56 (d, J=6.0 Hz, 1H), 6.49 (d, J=6.0 Hz, 1H), 6.31 (d, J=4.8 Hz, 1H), 4.22-4.16 (m, 1H), 3.98-3.86 (m, 7H), 3.75 (s, 3H), 3.66 (s, 3H), 3.64 (s, 3H), 3.57-3.52 (m, 2H), 3.31-3.14 (m, 3H), 2.78-2.68 (m, 2H), 2.43-2.29 (m, 1H), 1.82-1.77 (m, 4H), 1.59-1.52 (m, 2H).

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 40 | 60 | 30 |
| 2.00 | 40 | 60 | 30 |
| 18.00 | 95 | 5 | 30 |

### Preparation Example 24: Preparation of (S)-8-((5-(((2R,11aS)-2-hydroxy-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-1,10,11,11a-tetrahydro-5H-benzo[e]pyrrolo[1,2-a][1,4]diazepin-5-one (7-40)

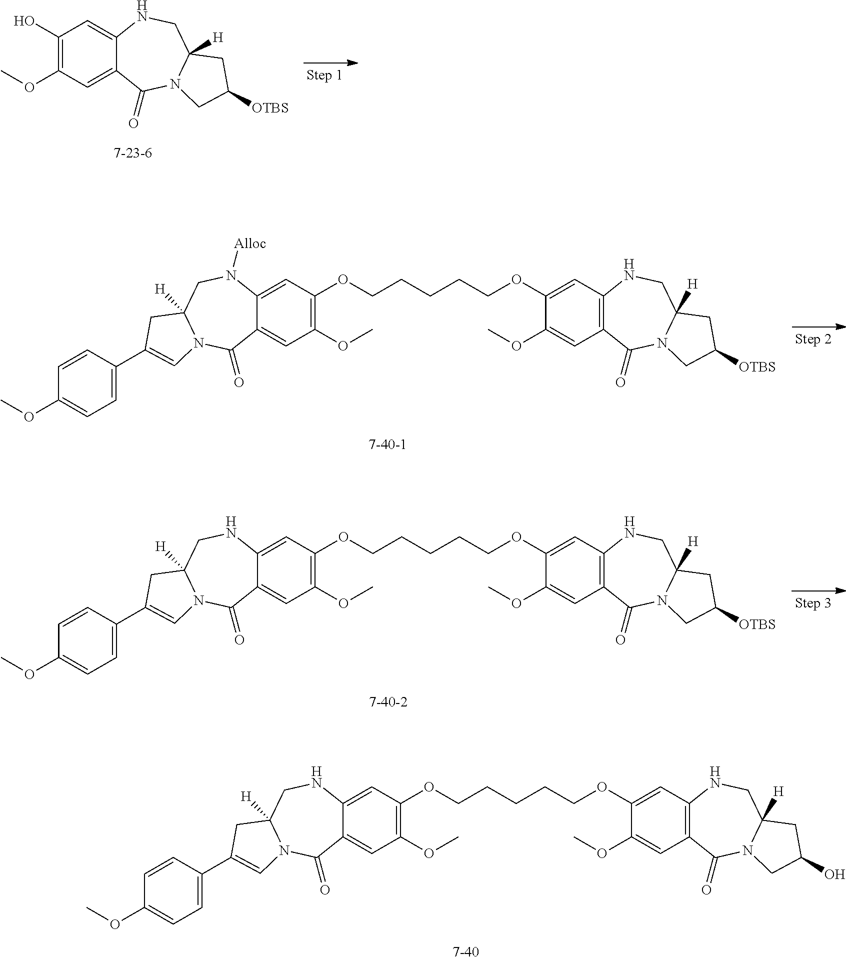

### Step 1: Preparation of allyl (S)-8-((5-(((2R,11aS)-2-((tert-butyldimethylsilyl)oxy)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-5-5oxo-11,11a-dihydro-1H-benzo[e]pyrrolo[1,2-a][1,4]diazepin-10(5H)-carboxylate (7-40-1)

Allyl (S)-8-((5-bromopentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (45 mg, 76.86 μmol) and (2R,11aS)-2-((tert-butyldimethylsilyl)oxy)-8-hydroxy-7-methoxy-1,2,3,10,11,11a-hexahydro-5H-benzo[e]pyrrolo[1,2-a][1,4]diazepin-5-one (34.91 mg, 92.23 μmol) were dissolved in DMF (1 mL), followed by an addition of potassium carbonate (21.24 mg, 153.72 μmol), and reacted under stirring for 16 hours. The reaction mixture was treated with water to precipitate solid, which was filtered, washed with water and dried to obtain a crude product (65 mg, 73.60 μmol), which was used directly in the next step without purification.

Its structural characterization data are as follows:

MS m/z (ESI): 883.1[M+H]⁺

### Step 2: Preparation of (S)-8-((5-(((2R,11aS)-2-((tert-butyldimethylsilyl)oxy)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-1,10,11,11a-tetrahydro-5H-benzo[e]pyrrolo[1,2-a][1,4]diazepin-5-one (7-40-2)

Allyl (S)-8-((5-(((2R,11aS)-2-((tert-butyldimethylsilyl)oxy)-7-methoxy-5-oxo-2,3,5,10,11, 11a-hexahydro-1H-benzo[e]pyrrolo[1,2-a][1,4]diazepin-8-yl)oxy)pentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (65 mg, 73.60 μmol) was dissolved in DMF (1 mL), followed by an addition of tetrakis(triphenylphosphine)palladium (8.51 mg, 7.36 μmol) and tetrahydropyrrole (5.23 mg, 73.60 μmol), subjected to nitrogen replacement, and reacted under stirring and protection for 12 hours. The reaction mixture was treated with ethyl acetate and water, stirred, filtered, and allowed to stand for liquid separation. The organic phase was washed 3 times with saturated brine, dried, and concentrated under reduced pressure to obtain a crude product (58 mg, 73.60 μmol), which was used directly in the next step without purification.

Its structural characterization data are as follows:

MS m/z (ESI): 800.5[M+H]⁺

### Step 3: Preparation of (S)-8-((5-(((2R,11aS)-2-hydroxy-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-1,10,11,11a-tetrahydro-5H-benzo[e]pyrrolo[1,2-a][1,4]diazepin-5-one (7-40)

(S)-8-((5-(((2R,11aS)-2-((Tert-butyldimethylsilyl)oxy)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-1,10,11,11a-tetrahydro-5H-benzo[e]pyrrolo[1,2-a][1,4]diazepin-5-one (58 mg, 73.60 μmol) was dissolved in tetrahydrofuran (1 mL), followed by a dropwise addition of tetrabutylammonium fluoride solution (1 M, 145.17 μL), and reacted under stirring for 1 hour. The reaction mixture was treated with ethyl acetate for dilution, washed twice with saturated ammonium chloride aqueous solution and twice with saturated brine, and concentrated under reduced pressure to obtain a crude product, which was purified by preparative HPLC and freeze-dried to obtain the title compound (17.11 mg, 24.24 μmol).

Its structural characterization data are as follows:

MS m/z (ESI): 685.3[M+H]⁺

¹H NMR (400 MHz, DMSO) δ 7.45 (s, 1H), 7.42-7.36 (m, 3H), 7.29 (s, 1H), 6.91 (d, J=8.9 Hz, 2H), 6.55 (d, J=5.7 Hz, 1H), 6.36 (d, J=5.9 Hz, 1H), 6.31 (s, 1H), 6.26 (s, 1H), 4.92 (d, J=3.2 Hz, 1H), 4.19 (s, 2H), 3.93 (dd, J=13.7, 6.7 Hz, 4H), 3.80 (d, J=9.1 Hz, 1H), 3.76 (s, 3H), 3.65 (d, J=9.5 Hz, 6H), 3.51 (dt, J=12.1, 9.0 Hz, 4H), 3.28-3.19 (m, 2H), 2.99-2.93 (m, 1H), 2.79-2.74 (m, 1H), 2.08 (dd, J=13.0, 6.7 Hz, 1H), 1.80 (s, 4H), 1.73-1.67 (m, 1H), 1.56 (d, J=6.4 Hz, 2H).

Its preparation method was as follows:
Chromatographic column: Waters SunFire Prep C18 OBD (5 μm*19 mm*150 mm)
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 28 |
| 4.00 | 30 | 70 | 28 |
| 24.00 | 90 | 10 | 28 |

### Preparation Example 25: Preparation of (S)-7-methoxy-8-((5-(((S)-7-methoxy-2-methylene-5-oxo-2,3,5,10,11,11a-hexahydro-1H-benzo[e]pyrrolo[1,2-a][1,4]diazepin-8-yl)oxy)pentyl)oxy)-2-(4-methoxyphenyl)-1,10,11,11a-tetrahydro-5H-benzo[e]pyrrolo[1,2-a][1,4]diazepin-5-one (7-41)

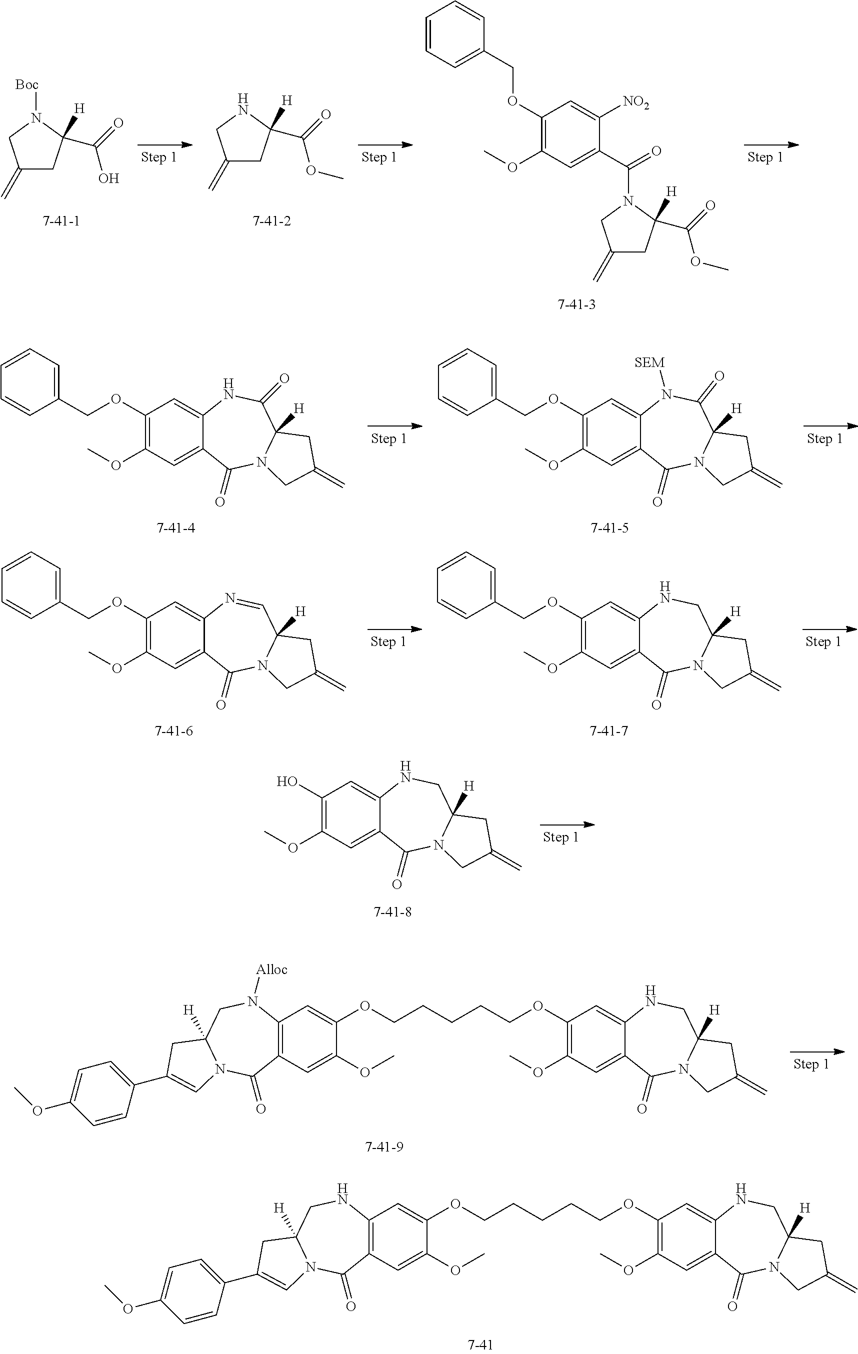

### Step 1: Preparation of methyl (S)-4-methylenepyrrolidine-2-carboxylate (7-41-2)

(S)-1-(Tert-butyloxycarbonyl)-4-methylenepyrrolidine-2-carboxylic acid (941.87 mg, 4.14 mmol) was dissolved in MeOH (7 mL), followed by a dropwise addition of HCl/dioxane (4M, 7 mL), and reacted under stirring at room temperature for 24 hours. After the reaction was completed, the reaction mixture was directly concentrated under reduced pressure to obtain a crude hydrochloride of the title compound (794 mg, 4.02 mmol), which was directly used in the next step without purification.

Its structural characterization data are as follows:

ESI-MS (m/z): 142.2[M+H]⁺

### Step 2: Preparation of methyl (S)-1-(4-(benzyloxy)-5-methoxy-2-nitrobenzoyl)-4-methylenepyrrolidine-2-carboxylate (7-41-3)

4-(Benzyloxy)-5-methoxy-2-nitrobenzoic acid (1.35 g, 4.45 mmol) was dissolved in DMF (15 mL), followed by an addition of HATU (2.03 g, 5.34 mmol) and DIPEA (1.72 g, 13.34 mmol, 2.32 mL), stirred at room temperature for 10 minutes, then a mixed solution of methyl (S)-4-methylenepyrrolidine-2-carboxylate hydrochloride (790 mg, 4.45 mmol) and DIPEA (574.80 mg, 4.45 mmol) in DMF (15 mL) was added to the reaction mixture. After the addition was completed, the reaction was carried out under stirring at room temperature for 2 hours. After the reaction was completed, 100 mL of water was added to the reaction solution, and extracted with ethyl acetate (50 mL×3). The organic phases were combined, washed with brine, dried over anhydrous sodium sulfate, and concentrated under reduced pressure to obtain a crude product, which was purified by silica gel column chromatography (ethyl acetate/petroleum ether=0 to 60%) and concentrated under reduced pressure again to obtain the title compound (1.53 g, 3.58 mmol).

Its structural characterization data are as follows:

ESI-MS (m/z): 427.4[M+H]⁺

### Step 3: Preparation of (S)-8-(benzyloxy)-7-methoxy-2-methylene-1,2,3,11a-tetrahydro-5H-benzo[e]pyrrolo[1,2-a][1,4]diazepin-5,11(10H)-dione (7-41-4)

Methyl (S)-1-(4-(benzyloxy)-5-methoxy-2-nitrobenzoyl)-4-methylenepyrrolidine-2-carboxylate (1.53 g, 3.59 mmol) was dissolved in saturated aqueous ammonium chloride solution (8 mL) and methanol (80 mL), followed by an addition of zinc powder (2.35 g, 35.88 mmol), heated to 75° C. and reacted under stirring for 4 hours. After the reaction was completed, the reaction mixture was cooled to room temperature and filtered, and the filtrate was concentrated under reduced pressure. 50 ml of water was added to the residue, and extracted with a mixed solvent of ethyl acetate:methanol=4:1 (50 ml×3). The organic phases were combined, washed with saturated brine, dried over anhydrous sodium sulfate and concentrated under reduced pressure to obtain a crude product of the title compound (1.3 g, 3.57 mmol), which was directly used in the next reaction without purification.

Its structural characterization data are as follows:

ESI-MS (m/z): 365.2 [M+H]⁺

### Step 4: Preparation of (S)-8-(benzyloxy)-7-methoxy-2-methylene-10-((2-(trimethylsilyl) ethoxy)methyl)-1,2,3,11a-tetrahydro-5H-benzo[e]pyrrolo[1,2-a][1,4]diazepin-5,11(10H)-dione (7-41-5)

(S)-8-(Benzyloxy)-7-methoxy-2-methylene-1,2,3,11a-tetrahydro-5H-benzo[e]pyrrolo[1,2-a][1,4]diazepin-5,11(10H)-dione (0.2 g, 548.86 μmol) was dissolved in THF (3 mL), cooled to −40° C. under nitrogen protection, followed by an addition of n-BuLi (2.5 M, 230.52 L). After the addition was completed, the reaction was carried out under thermal insulation conditions for 30 minutes. Then 2-(trimethylsilyl)ethoxymethyl chloride (100.66 mg, 603.74 μmol) was added dropwise at −40° C. After the addition completed, the reaction mixture was heated naturally to room temperature and reacted overnight. After the reaction was completed, the reaction was quenched with 5 mL of ammonium chloride aqueous solution, 10 mL of water was added, and extracted with ethyl acetate (10 mL×3). The organic phases were combined, washed with saturated brine, and dried over anhydrous sodium sulfate, and then concentrated under reduced pressure to obtain a crude product of the title compound (286 mg, 520.36 μmol), which was directly used in the next reaction without purification.

Its structural characterization data are as follows:

ESI-MS (m/z): 496.3 [M+H]⁺

### Step 5: Preparation of (S)-8-(benzyloxy)-7-methoxy-2-methylene-1,2,3,11a-tetrahydro-5H-benzo[e]pyrrolo[1,2-a][1,4]diazepin-5-one (7-41-6)

(S)-8-(Benzyloxy)-7-methoxy-2-methylene-1,2,3,11a-tetrahydro-5H-benzo[e]pyrrolo[1,2-a][1,4]diazepin-5-one (180 mg, 363.89 μmol) was dissolved in THF (10 mL), cooled to −78° C. under nitrogen protection, followed by a dropwise addition of lithium triethylborohydride (1 M, 727.78 L), and reacted under thermal insulation conditions for 2 hours. Lithium triethylborohydride (1 M, 727.78 μL) was added three times, each time with an interval of 1 hour. After the reaction was completed, the reaction mixture was added to 15 mL of water, extracted with ethyl acetate three times (10 mL×3). The organic phases were combined, washed with saturated brine, dried over anhydrous sodium sulfate, and concentrated under reduced pressure to obtain a crude product of the title compound (200 mg, 344.44 μmol), which was directly used in the next step without purification.

Its structural characterization data are as follows:

ESI-MS (m/z): 367.3 [M+H+H₂O]⁺; 697.4 [2M+H]⁺

### Step 6: Preparation of (S)-8-(Benzyloxy)-7-methoxy-2-methylene-1,2,3,10,11,11a-hexahydro-5H-benzo[e]pyrrolo[1,2-a][1,4]diazepin-5-one (7-41-7)

(S)-8-(Benzyloxy)-7-methoxy-2-methylene-1,2,3,11a-tetrahydro-5H-benzo[e]pyrrolo[1,2-a][1,4]diazepin-5-one (80 mg, 229.62 μmol) was dissolved in dichloromethane (1 mL) and methanol (1 mL), followed by an addition of sodium triacetoxyborohydride (97.33 mg, 459.25 μmol), stirred at room temperature overnight for 16 hours. NaBH₄ (17.37 mg, 459.25 μmol) was added, reacted for 2 hours, and then NaBH₄ (17.37 mg, 459.25 μmol) was added again and reacted for 2 hours. After the reaction was completed, the reaction mixture was concentrated under reduced pressure, dissolved in methanol, purified by reverse phase column chromatography (acetonitrile/0.05% formic acid aqueous solution=0 to 50%), and freeze-dried to obtain the title compound (15 mg, 42.81 μmol).

Its structural characterization data are as follows:

ESI-MS (m/z): 351.3 [M+H]⁺

### Step 7: Preparation of (S)-8-hydroxy-7-methoxy-2-methylene-1,2,3,10,11,11a-hexahydro-5H-benzo[e]pyrrolo[1,2-a][1,4]diazepin-5-one (7-41-8)

(S)-8-(Benzyloxy)-7-methoxy-2-methylene-1,2,3,10,11,11a-hexahydro-5H-benzo[e]pyrrolo[1,2-a][1,4]diazepin-5-one (15 mg, 42.81 μmol) was added in dichloromethane (1 mL), followed by a dropwise addition of methanesulfonic acid (0.1 mL), and reacted under stirring at room temperature for 1 hour. After the reaction was completed, the reaction mixture was treated with 2 mL of water, adjusted to pH=8-9 with saturated sodium bicarbonate aqueous solution, and extracted with dichloromethane (3 mL×3). The organic phases were combined, washed with 3 mL of saturated brine, dried over anhydrous sodium sulfate, and concentrated under reduced pressure to obtain a crude product of the title compound (11 mg, 42.26 μmol), which was directly used in the next step without purification.

Its structural characterization data are as follows:

ESI-MS (m/z): 261.1 [M+H]⁺

### Step 8: Preparation of allyl (S)-7-methoxy-8-((5-(((S)-7-methoxy-2-methylene-5-oxo-2,3,5,10,11,11a-hexahydro-1H-benzo)[e]pyrrolo[1,2-a][1,4]diazepin-8-yl)oxy)pentyl)oxy)-2-(4-methoxyphenyl)-5-oxo-11,11a-dihydro-1H-benzo[e]pyrrolo[1,2-a][1,4]diazepin-10(5H)-carboxylate (7-41-9)

Allyl (S)-8-((5-bromopentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (24.74 mg, 42.26 μmol) and (S)-8-hydroxy-7-methoxy-2-methylene-1,2,3,10,11,11a-hexahydro-5H-benzo[e]pyrrolo[1,2-a][1,4]diazepin-5-one (11 mg, 42.26 μmol) were dissolved in DMF (1 mL), followed by an addition of potassium carbonate (17.52 mg, 126.78 μmol), and reacted under stirring at room temperature for 16 hours. After the reaction was completed, the reaction mixture was directly purified by preparative high performance liquid chromatography and freeze-dried to obtain the title compound (5 mg, 6.54 μmol).

Its structural characterization data are as follows:

ESI-MS (m/z): 766.3 [M+H]⁺

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 28 |
| 2.00 | 30 | 70 | 28 |
| 18.00 | 90 | 10 | 28 |

### Step 9: Preparation of (S)-7-methoxy-8-((5-(((S)-7-methoxy-2-methylene-5-oxo-2,3,5,10, 11,11a-hexahydro-1H-benzo[e]pyrrolo[1,2-a][1,4]diazepin-8-yl)oxy)pentyl)oxy)-2-(4-methoxyphenyl)-1,10,11,11a-tetrahydro-5H-benzo[e]pyrrolo[1,2-a][1,4]diazepin-5-one (7-41)

Allyl (S)-7-methoxy-8-((5-(((S)-7-methoxy-2-methylene-5-oxo-2,3,5,10,11,11a-hexahydro-1H-benzo)[e]pyrrolo[1,2-a][1,4]diazepin-8-yl)oxy)pentyl)oxy)-2-(4-methoxyphenyl)-5-oxo-11,11a-dihydro-1H-benzo[e]pyrrolo[1,2-a][1,4]diazepin-10(5H)-carboxylate (5 mg, 6.54 μmol) was dissolved in DMF (0.5 mL), followed by an addition of tetrahydropyrrole (464.92 μg, 6.54 μmol) and tetrakis(triphenylphosphine)palladium (755.41 μg, 6.54e-1 μmol), subjected to nitrogen replacement for three times, then reacted under stirring and nitrogen protection at room temperature for 2 hours. After the reaction was completed, the reaction mixture was directly purified by preparative HPLC and freeze-dried to obtain the title compound (1.89 mg, 2.47 μmol).

Its structural characterization data are as follows:

ESI-MS (m/z): 681.4 [M+H]⁺

Its preparation method was as follows:
Chromatographic column: Waters SunFire Prep C18 OBD 19 mm×150 mm×5.0 μm
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 25 | 75 | 30 |
| 3.00 | 25 | 75 | 30 |
| 18.00 | 90 | 10 | 30 |

### Preparation Example 26: Preparation of (1aR,9aS,10aR)-5-methoxy-6-((5-(((S)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-1a,8,9,9a,10,10a-hexahydrobenzo[e]cyclopropo[4,5]pyrrolo[1,2-a][1,4]diazepin-3(1H)-one (7-43)

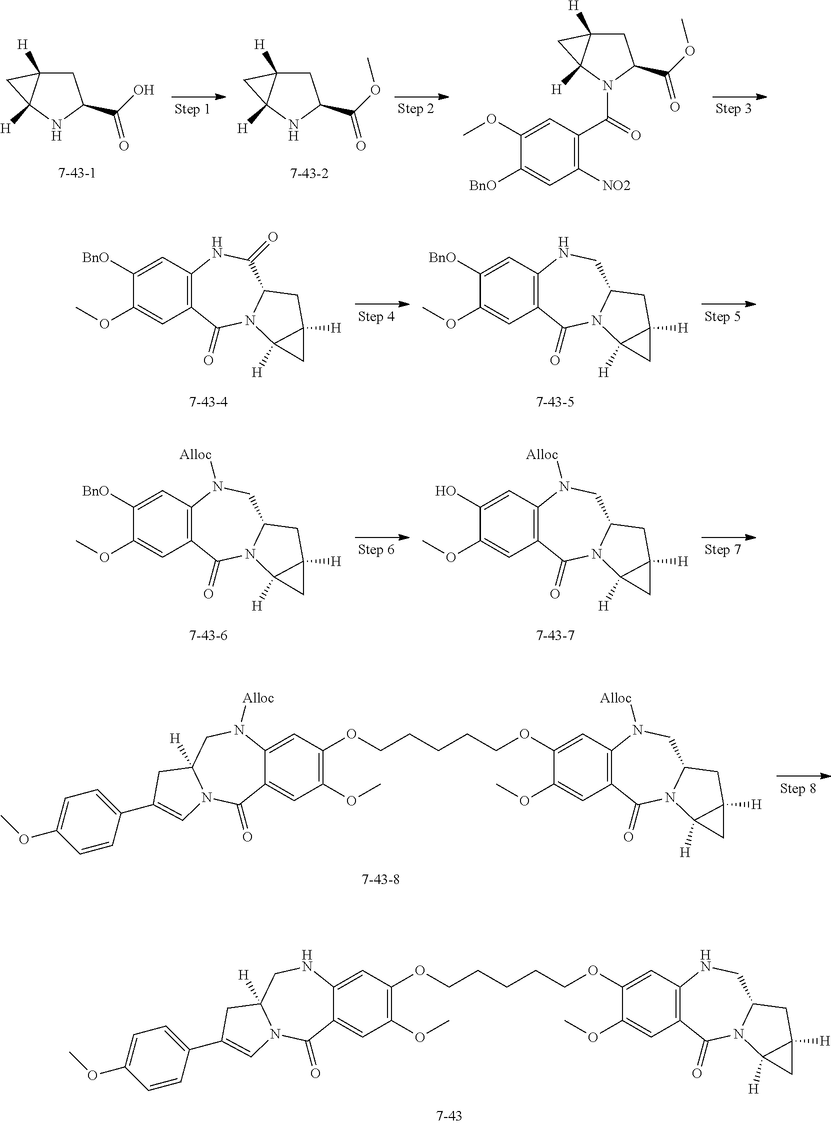

### Step 1: Preparation of methyl (1R,3S,5R)-2-azabicyclo[3.1.0]hexan-3-carboxylate Hydrochloride (7-43-2)

(1R,3S,5R)-2-Azabicyclo[3.1.0]hexan-3-carboxylic acid (2.00 g, 8.80 mmol) was dissolved in methanol (10 mL), followed by an addition of 4M hydrochloric acid dioxane solution (10 mL), and reacted under stirring for 4 hours. The reaction mixture was concentrated under reduce pressure, and directly used in the next step.

### Step 2: Preparation of methyl (1R,3S,5R)-2-(4-(benzyloxy)-5-methoxy-2-nitrobenzoyl)-2-azabicyclo[3.1.0]hexan-3-carboxylate (7-43-3)

4-Benzyloxy-5-methoxy-2-nitro-benzoic acid (1.18 g, 3.89 mmol), methyl (1R,3S,5R)-2-azabicyclo[3.1.0]hexan-3-carboxylate hydrochloride (760.26 mg, 4.28 mmol) and HATU (2.22 g, 5.84 mmol) were dissolved in DMF (20 mL), followed by a dropwise addition of DIPEA (1.51 g, 11.67 mmol), and reacted under stirring for 2 hours. The reaction mixture was treated with water and EA, stirred, and allowed to stand for liquid separation. The aqueous phase was extracted with EA twice. The organic phases were combined, washed twice with saturated brine, dried, concentrated, purified by silica gel column (eluent 30-65% ethyl acetate/petroleum ether) and concentrated again to obtain the title compound (1.64 g, 3.85 mmol).

### Step 3: Preparation of (1aR,9aS,10aR)-6-(benzyloxy)-5-methoxy-1a,9a,10,10a-tetrahydrobenzo[e]cyclopropo[4,5]pyrrolo[1,2-a][1,4]diazepin-3,9(1H,8H)-dione (7-43-4)

Methyl (1R,3S,5R)-2-(4-(benzyloxy)-5-methoxy-2-nitrobenzoyl)-2-azabicyclo[3.1.0]hexan-3-carboxylate (870 mg, 2.04 mmol) was dissolved in methanol (10 mL), followed by an addition of saturated aqueous ammonium chloride solution (2 mL) and zinc powder (1.33 g, 20.40 mmol) in batches, heated to 80° C. and reacted under refluxing for 16 hours. The reaction mixture was cooled to room temperature, filtered, and the filtrate was concentrated under reduced pressure, purified by silica gel column (eluent: 5% methanol/dichloromethane) and concentrated again to obtain the title compound (682 mg, 1.87 mmol).

Its structural characterization data are as follows:

MS m/z (ESI): 365.1[M+H]⁺

### Step 4: Preparation of (1aR,9aS,10aR)-6-(benzyloxy)-5-methoxy-1a,9a,10,10a-tetrahydrobenzo[e]cyclopropo[4,5]pyrrolo[1,2-a][1,4]diazepin-3,9(1H,8H)-dione (7-43-5)

(1aR,9aS,10aR)-6-(Benzyloxy)-5-methoxy-1a,9a,10,10a-tetrahydrobenzo[e]cyclopropo[4,5]pyrrolo[1,2-a][1,4]diazepin-3,9(1H,8H)-dione (180 mg, 493.97 μmol) was dissolved in dry THF (10 mL), followed by an addition of sodium borohydride (56.06 mg, 1.48 mmol) under stirring and a dropwise addition of trifluoroacetic acid (112.65 mg, 987.94 μmol, 75.65 μL) in tetrahydrofuran solution (100 μL) to generate a large number of bubbles. The reaction mixture was heated to 75° C. and reacted under stirring for 4 hours, then concentrated under reduced pressure, followed by an addition of water and ethyl acetate, stirred, and allowed to stand for liquid separation. The organic phase was concentrated under reduced pressure, purified by silica gel column (eluent: 5% methanol/dichloromethane), and concentrated again to obtain the title compound (142 mg, 405.24 μmol).

Its structural characterization data are as follows:

MS m/z (ESI): 367.3[M+H₂O]⁺

### Step 5: Preparation of allyl (1aR,9aS,10aR)-6-(benzyloxy)-5-methoxy-3-oxo-1,1a,9,9a,10, 10a-hexahydrobenzo[e]cyclopropo[4,5]pyrrolo[1,2-a][1,4]diazepin-8(3H)-carboxylate (7-43-6)

(1aR,9aS,10aR)-6-(Benzyloxy)-5-methoxy-1a,9a,10,10a-tetrahydrobenzo[e]cyclopropo[4,5]pyrrolo[1,2-a][1,4]diazepin-3,9(1H,8H)-dione (142 mg, 405.24 μmol) was dissolved in dry dichloromethane (5 mL), pyridine (48.08 mg, 607.86 μmol) was added dropwise, cooled under stirring to 0° C., followed by a dropwise addition of allyl chlorate (195.38 mg, 1.62 mmol, 172.29 μL), then heated to room temperature and reacted under stirring for 1 hour. The reaction mixture was diluted with dichloromethane, followed by an addition of 10% citric acid aqueous solution, stirred, and allowed to stand for liquid separation. The organic phase was washed with water, dried, concentrated, purified by silica gel column (eluent: 80% ethyl acetate/petroleum ether) and concentrated again to obtain the title compound (153 mg, 352.14 μmol).

Step 6: Preparation of allyl (1aR,9aS,10aR)-6-hydroxy-5-methoxy-3-oxo-1,1a,9,9a,10,10a-hexahydrobenzo[e]cyclopropo[4,5]pyrrolo[1,2-a][1,4]diazepin-8(3H)-carboxylate (7-43-7)

Allyl (1aR,9aS,10aR)-6-(benzyloxy)-5-methoxy-3-oxo-1,1a,9,9a,10,10a-hexahydrobenzo[e]cyclopropo[4,5]pyrrolo[1,2-a][1,4]diazepin-8(3H)-carboxylate (130 mg, 299.21 μmol) was dissolved in dichloromethane (1 mL), followed by a dropwise addition of methanesulfonic acid (0.5 mL) under stirring, and reacted continuously for 1 hour. The reaction mixture was diluted with dichloromethane, followed by an addition of sodium bicarbonate aqueous solution, stirred, and allowed to stand for liquid separation. The organic phase was dried, concentrated, purified by preparative high performance liquid chromatography and freeze-dried to obtain the title compound (57 mg, 165.52 μmol).

Its structural characterization data are as follows:

MS m/z (ESI): 345.1[M+H]⁺

Its preparation method was as follows:
Chromatographic column: Waters SunFire Prep C18 OBD (5 μm*19 mm*150 mm)
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 28 |
| 2.00 | 30 | 70 | 28 |
| 22.00 | 90 | 10 | 28 |

### Step 7: Preparation of allyl (1aR,9aS,10aR)-6-((5-(((S)-10-((allyloxy)carbonyl)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-5-methoxy-3-oxo-1,1a,9,9a,10,10a-hexahydrobenzo[e]cyclopropo[4,5]pyrrolo[1,2-a][1,4]diazepin-8(3H)-carboxylate (7-43-8)

Allyl (S)-8-((5-bromopentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (10.20 mg, 17.42 μmol), allyl (1aR,9aS,10aR)-6-hydroxy-5-methoxy-3-oxo-1,1a,9,9a,10,10a-hexahydrobenzo[e]cyclopropo[4,5]pyrrolo[1,2-a][1,4]diazepin-8(3H)-carboxylate (5 mg, 14.52 μmol) and potassium carbonate (3.01 mg, 21.78 μmol) were dissolved in DMF (1 mL), and reacted under stirring for 16 hours. The reaction mixture was treated with water to precipitate solids, which was filtered, washed with water and dried in vacuum to obtain the title compound (12 mg, 14.14 μmol).

Its structural characterization data are as follows:

MS m/z (ESI): 849.4[M+H]⁺

### Step 8: Preparation of (1aR,9aS,10aR)-5-methoxy-6-((5-(((S)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-1a,8,9,9a,10,10a-hexahydrobenzo[e]cyclopropo[4,5]pyrrolo[1,2-a][1,4]diazepin-3(1H)-one (7-43)

Allyl (1aR,9aS,10aR)-6-((5-(((S)-10-((allyloxy)carbonyl)-7-methoxy-2-(4-methoxyphenyl)-5-oxo-5,10,11,11a-tetrahydro-1H-benzo[e]pyrrolo[1,2-a][1,4]diazepin-8-yl)oxy)pentyl)oxy)-5-methoxy-3-oxo-1,1a,9,9a,10,10a-hexahydrobenzo[e]cyclopropo[4,5]pyrrolo[1,2-a][1,4]diazepin-8(3H)-carboxylate (12 mg, 14.14 μmol) was dissolved in DMF (1 mL), followed by a dropwise addition of tetrahydropyrrole (1.01 mg, 14.14 μmol) and an addition of tetrakis(triphenylphosphine)palladium (1.63 mg, 1.41 μmol), subjected to nitrogen replacement, and reacted under protection for 16 hours. The product was purified by preparative HPLC and freeze-dried to obtain the title compound (6.18 mg, 8.99 μmol).

Its structural characterization data are as follows:

MS m/z (ESI): 681.3[M+H]⁺

Its preparation method was as follows:
Chromatographic column: Waters SunFire Prep C18 OBD (5 μm*19 mm*150 mm)
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 28 |
| 4.00 | 30 | 70 | 28 |
| 24.00 | 90 | 10 | 28 |

### Preparation Example 27: Preparation of (S)-7-methoxy-8-((5-(((S)-7-methoxy-5-oxo-2,3,5, 10,11,11a-hexahydro-1H-benzo[e]pyrrolo[1,2-a][1,4]diazepin-8-yl)oxy)pentyl)oxy)-2-(4-methoxyphenyl)-1,10,11,11a-tetrahydro-5H-benzo[e]pyrrolo[1,2-a][1,4]diazepin-5-one (7-44)

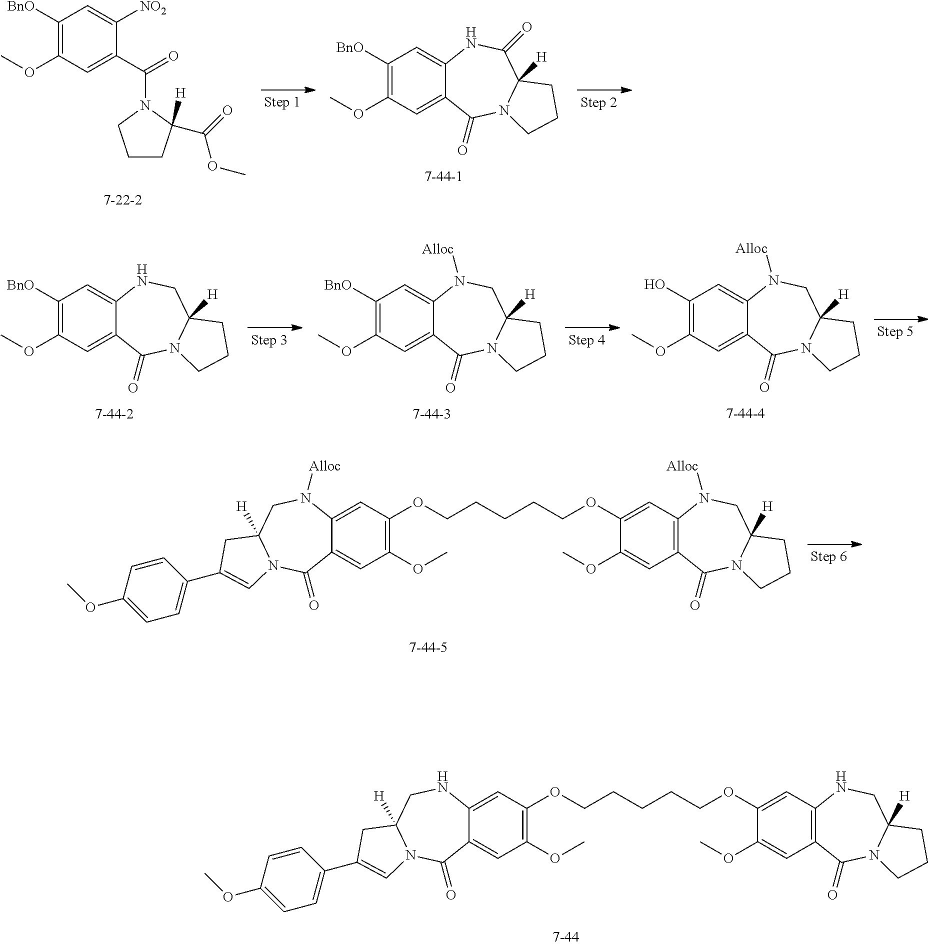

### Step 1: Preparation of (S)-8-(benzyloxy)-7-methoxy-1,2,3,11a-tetrahydro-5H-benzo[e]pyrrolo[1,2-a][1,4]diazepin-5,11(10H)-dione (7-44-1)

(4-(Benzyloxy)-5-methoxy-2-nitrobenzoyl)-L-proline methyl ester (870 mg, 2.04 mmol) was dissolved in methanol (100 mL), followed by an addition of saturated aqueous ammonium chloride solution (10 mL) and zinc powder (2.84 g, 43.44 mmol) in batches, heated to 80° C. and reacted under refluxing for 16 hours. The reaction mixture was cooled to room temperature, filtered, and the filtrate was concentrated under reduced pressure, purified by silica gel column (eluent: 5% methanol/dichloromethane) and concentrated again to obtain the title compound (1.15 g, 3.26 mmol).

Its structural characterization data are as follows:

MS m/z (ESI): 353.1[M+H]⁺

### Step 2: Preparation of (S)-8-(benzyloxy)-7-methoxy-1,2,3,10,11,11a-hexahydro-5H-benzo[e]pyrrolo[1,2-a][1,4]diazepin-5-one (7-44-2)

(S)-8-(Benzyloxy)-7-methoxy-1,2,3,11a-tetrahydro-5H-benzo[e]pyrrolo[1,2-a][1,4]diazepin-5,11(10H)-dione (1.1 g, 3.12 mmol) was dissolved in dry THF (10 mL), followed by an addition of sodium borohydride (354.29 mg, 9.36 mmol) under stirring and a dropwise addition of trifluoroacetic acid (711.85 mg, 6.24 mmol, 478.07 μL) in tetrahydrofuran solution (100 μL) to generate a large number of bubbles, heated to 75° C. and reacted under stirring for 4 hours. The reaction mixture was concentrated under reduced pressure, followed by an addition of water and ethyl acetate, stirred, and allowed to stand for liquid separation. The organic phase was concentrated under reduced pressure, purified by silica gel column (eluent: 5% methanol/dichloromethane), and concentrated again to obtain the title compound (1.01 g, 2.98 mmol).

### Step 3: Preparation of allyl (S)-8-(benzyloxy)-7-methoxy-5-oxo-2,3,11,11a-tetrahydro-1H-benzo[e]pyrrolo[1,2-a][1,4]diazepin-10(5H)-carboxylate (7-44-3)

(S)-8-(Benzyloxy)-7-methoxy-1,2,3,10,11,11a-hexahydro-5H-benzo[e]pyrrolo[1,2-a][1,4]diazepin-5-one (1 g, 2.96 mmol) was dissolved in dry dichloromethane (50 mL), followed by an addition of pyridine (350.62 mg, 4.43 mmol, 358.51 μL), cooled under stirring to 0° C., followed by a dropwise addition of allyl chlorate (1.07 g, 8.87 mmol, 942.30 μL), heated to room temperature and reacted under stirring for 1 hour. The reaction mixture was diluted with dichloromethane, followed by an addition of 10% citric acid aqueous solution, stirred, and allowed to stand for liquid separation. The organic phase was washed with water, dried, concentrated, purified by silica gel column (eluent: 80% ethyl acetate/petroleum ether) and concentrated again to obtain the title compound (530 mg, 1.25 mmol).

### Step 4: Preparation of allyl (S)-8-hydroxy-7-methoxy-5-oxo-2,3,11,11a-tetrahydro-1H-benzo[e]pyrrolo[1,2-a][1,4]diazepin-10(5H)-carboxylate (7-44-4)

Allyl (S)-8-(benzyloxy)-7-methoxy-5-oxo-2,3,11,11a-tetrahydro-1H-benzo[e]pyrrolo[1,2-a][1,4]diazepin-10(5H)-carboxylate (530 mg, 1.25 mmol) was dissolved in dichloromethane (4 mL), followed by a dropwise addition of methanesulfonic acid (2 mL) under stirring, and reacted continuously for 1 hour. The reaction mixture was diluted with dichloromethane, followed by an addition of sodium bicarbonate aqueous solution, stirred, and allowed to stand for liquid separation. The organic phase was dried and concentrated to obtain the title compound (410 mg, 1.23 mmol).

Its structural characterization data are as follows:

MS m/z (ESI): 333.2[M+H]⁺

### Step 5: Preparation of allyl (S)-8-((5-(((S)-10-((allyloxy)carbonyl)-7-methoxy-5-oxo-2,3,5, 10,11,11a-hexahydro-1H-benzo[e]pyrrolo[1,2-a][1,4]diazepin-8-yl)oxy)pentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (7-44-5)

Allyl (S)-8-((5-bromopentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (40 mg, 68.32 μmol), allyl (S)-8-hydroxy-7-methoxy-5-oxo-2,3,11,11a-tetrahydro-1H-benzo[e]pyrrolo[1,2-a][1,4]diaza-10(5H)-carboxylate (22.71 mg, 68.32 μmol) and potassium carbonate (14.16 mg, 102.48 μmol) were dissolved in DMF (1 mL), and reacted under stirring for 16 hours. The reaction mixture was treated with water to precipitate solid, which was filtered, washed with water and dried in vacuum to obtain the title compound (55 mg, 65.72 μmol).

Its structural characterization data are as follows:

MS m/z (ESI): 838.4[M+H]⁺

### Step 6: Preparation of (S)-7-methoxy-8-((5-(((S)-7-methoxy-5-oxo-2,3,5,10,11,11a-hexahydro-1H-benzo[e]pyrrolo[1,2-a][1,4]diazepin-8-yl)oxy)pentyl)oxy)-2-(4-methoxyphenyl)-1,10,11,11a-tetrahydro-5H-benzo[e]pyrrolo[1,2-a][1,4]diazepin-5-one (7-44)

Allyl (S)-8-((5-(((S)-10-((allyloxy)carbonyl)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-2-(4-methoxyphenyl)-5-oxo-11,11a-dihydro-1H-benzo[e]pyrrolo[1,2-a][1,4]diazepin-10(5H)-carboxylate (55 mg, 65.72 μmol) was dissolved in DMF (1 mL), followed by a dropwise addition of tetrahydropyrrole (4.67 mg, 65.72 μmol) and tetrakis(triphenylphosphine)palladium (7.59 mg, 6.57 μmol), subjected to nitrogen replacement, and reacted under nitrogen protection for 16 hours. The product was purified by preparative high performance liquid chromatography and freeze-dried to obtain the title compound (11.71 mg, 17.33 μmol).

Its structural characterization data are as follows:

MS m/z (ESI): 669.3[M+H]⁺

¹H NMR (400 MHz, CDCl₃) δ 7.49 (s, 2H), 7.31 (d, J=8.7 Hz, 2H), 7.26 (s, 3H), 6.89 (d, J=8.8 Hz, 2H), 4.29 (s, 1H), 3.99 (s, 4H), 3.87 (s, 5H), 3.81 (d, J=11.6 Hz, 5H), 3.70 (s, 1H), 3.58 (d, J=10.2 Hz, 4H), 3.32 (s, 3H), 2.74 (dd, J=16.1, 3.6 Hz, 1H), 2.21 (dd, J=12.8, 6.3 Hz, 1H), 1.90 (m, 6H), 1.65 (m, 4H).

Its preparation method was as follows:
Chromatographic column: Waters SunFire Prep C18 OBD (5 μm*19 mm*150 mm)
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 40 | 60 | 28 |
| 4.00 | 40 | 60 | 28 |
| 24.00 | 90 | 10 | 28 |

### Preparation Example 28: Preparation of (11aS,11a′S)-8,8′-(pentane-1,5-diylbis(oxy))bis(7-methoxy-1,2,3,10,11,11a-hexahydro-5H-benzo[e]pyrrolo[1,2-a][1,4]diazepin-5-one) (7-45)

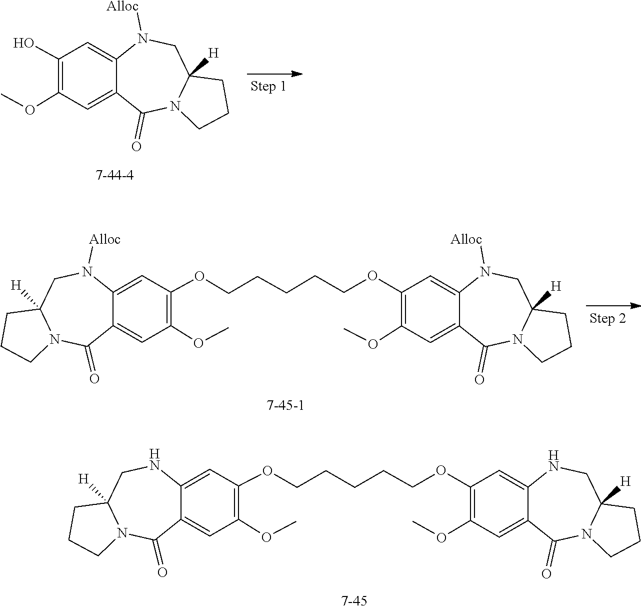

### Step 1: Preparation of allyl (S)-8-((5-(((R)-10-((allyloxy)carbonyl)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-5-oxo-2,3,11,11a-tetrahydro-1H-benzo[e]pyrrolo[1,2-a][1,4]diazepin-10(5H)-carboxylate (7-45-1)

1,5-Dibromopentane (12.45 mg, 54.16 μmol), allyl (S)-8-hydroxy-7-methoxy-5-oxo-2,3,11,11a-tetrahydro-1H-benzo[e]pyrrolo[1,2-a][1,4]diazepin-10(5H)-carboxylate (40 mg, 120.35 μmol) and potassium carbonate (49.90 mg, 361.06 μmol) were dissolved in DMF (1 mL), and reacted under stirring for 16 hours. The reaction mixture was treated with water to precipitate solid, which was filtered, washed with water and dried in vacuum to obtain the title compound (40 mg, 54.58 μmol).

Its structural characterization data are as follows:

MS m/z (ESI): 733.4[M+H]⁺

### Step 2: Preparation of (11aS,11a′S)-8,8′-(pentane-1,5-diylbis(oxy))bis(7-methoxy-1,2,3,10, 11,11a-hexahydro-5H-benzo[e]pyrrolo[1,2-a][1,4]diazepin-5-one) (7-45)

Allyl (S)-8-((5-(((R)-10-((allyloxy))carbonyl)-7-methoxy-5-oxo-2,3,5,10,11,11a-hexahydro-1H-benzo[e]pyrrolo[1,2-a][1,4]diazepin-8-yl)oxy)pentyl)oxy)-7-methoxy-5-oxo-2,3,11,11a-tetrahydro-1H-benzo[e]pyrrolo[1,2-a][1,4]diazepin-10(5H)-carboxylate (40 mg, 54.58 μmol) was dissolved in DMF (1 mL), followed by a dropwise addition of tetrahydropyrrole (3.88 mg, 54.58 μmol) and an addition of tetrakis(triphenylphosphine)palladium (6.31 mg, 5.46 μmol), subjected to nitrogen replacement, and reacted under nitrogen protection for 16 hours. The product was purified by preparative HPLC and freeze dried to obtain the title compound (4.96 mg, 8.70 μmol).

Its structural characterization data are as follows:

MS m/z (ESI): 565.3[M+H]⁺

¹H NMR (400 MHz, CDCl₃) δ 7.52 (s, 2H), 6.13 (s, 2H), 3.99 (s, 4H), 3.95-3.73 (m, 9H), 3.70 (s, 2H), 3.57 (d, J=11.7 Hz, 2H), 3.29 (s, 3H), 2.22 (dd, J=12.6, 6.1 Hz, 2H), 1.90 (dt, J=13.4, 6.5 Hz, 8H), 1.68 (d, J=20.9 Hz, 6H).

Its preparation method was as follows:
Chromatographic column: Waters SunFire Prep C18 OBD (5 μm*19 mm*150 mm)
Mobile phase A: acetonitrile; Mobile phase B: water (0.05% formic acid)

| Time [min] | Mobile phase A [%] | Mobile phase B [%] | Flow rate [mL/min] |
|---|---|---|---|
| 0.00 | 30 | 70 | 28 |
| 4.00 | 30 | 70 | 28 |
| 24.00 | 90 | 10 | 28 |

### Test Example: Inhibitory Effects of Compounds on Tumor Cell Proliferation

### 1. Inhibitory Effects of Compounds on NCI-N87 Cell Proliferation

(1) Cell plating: First, tumor cells NCI-N87 were cultured in the corresponding culture medium, the cells were digested with trypsin, and the cells were resuspended after centrifugation and counted. The cells were adjusted to an appropriate concentration for plating. The source of tumor cells is shown in Table 1.

TABLE 1
Source of tumor cells

| Cell name | Tumor type | Source |
|---|---|---|
| NCI-N87 | Human gastric | ATCC |
| | cancer cells | |

Co-incubation of the compound of the present invention and tumor cells: After the cells adhered, the culture medium was removed from the cell culture plates, and the diluted biologically active molecule (the compound of the present invention) was added to the wells of the above plate, and incubated for 72 hours.

Detection of in vitro cell activity: After the incubation was completed, 50 μL of Cell Counting-Lite™ 2.0 reagent (Vazyme) was added to each well, shaken and mixed well in the dark to perform the reaction for 10 minutes, then the detection was carried out, and a microplate reader (manufacturer: BMG, model: PHERAStar-FS) was used for reading. The background RLU was obtained using Cell Counting-Lite™ reagent in cell-free culture medium, and the solvent RLU was obtained using Cell Counting-Lite™ reagent in cell-containing culture medium. Cell inhibition rate=1−(sample RLU−background RLU)/(solvent RLU−background RLU)×100%, and the half maximal inhibitory concentration (IC₅₀) of the compound was calculated according to the four-parameter model fitting curve. RLU: relative light unit. The detection results are shown in Table 2-1 and Table 2-2.

### (2) Data Results

TABLE 2-1

| NCI-N87 cell proliferation inhibitory activity | |
|---|---|
| Compound number | IC₅₀ (nM) |
| 7-5 | 3.2 |
| 7-9 | 29.7 |
| 7-36 | 15.4 |
| 7-37 | 24.2 |
| 7-38 | 49.7 |
| 7-41 | 12.7 |

TABLE 2-2
NCI-N87 cell proliferation inhibitory activity

| Compound number | IC₅₀ (nM) |
|---|---|
| 7-5 | 4.1 |
| 7-3 | 79.3 |
| 7-29 | 117.4 |
| 7-34 | 120.2 |
| 7-35 | 117.9 |
| 7-39 | 11.0 |
| 7-40 | 93.6 |
| 7-42 | 100.1 |
| 7-43 | 10.1 |

The detection results showed that the compounds of the present invention had significant inhibitory effects on the proliferation of NCI-N87 human gastric cancer cells.

### 2. Inhibitory Effect of Compounds on HT29 Cell Proliferation

(1) Cell plating: First, tumor cells HT29 were cultured in the corresponding culture medium, the cells were digested with trypsin, and the cells were resuspended after centrifugation and counted. The cells were adjusted to an appropriate concentration for plating. The source of tumor cells is shown in Table 3.

TABLE 3
Source of tumor cells

| Cell name | Tumor type | Source |
|---|---|---|
| HT29 | Human colon | Cell Bank of Chinese |
| | cancer cells | Academy of Sciences |

Co-incubation of the compound of the present invention and tumor cells: After the cells adhered, the culture medium was removed from the cell culture plates, and the diluted biologically active molecule (the compound of the present invention) was added to the wells of the above plate, and incubated for 72 hours.

Detection of in vitro cell activity: After the incubation was completed, 50 μL of Cell Counting-Lite™ 2.0 reagent (Vazyme) was added to each well, shaken and mixed well in the dark to perform the reaction for 10 minutes, then the detection was carried out, and a microplate reader (manufacturer: BMG, model: PHERAStar-FS) was used for reading. The background RLU was obtained using Cell Counting-Lite™ reagent in cell-free culture medium, and the solvent RLU was obtained using Cell Counting-Lite™ reagent in cell-containing culture medium. Cell inhibition rate=1−(sample RLU−background RLU)/(solvent RLU−background RLU)×100%, and the half maximal inhibitory concentration (IC₅₀) of the compound was calculated according to the four-parameter model fitting curve. RLU: relative light unit. The detection results are shown in Table 4-1 and Table 4-2.

### (2) Data Results

TABLE 4-1
HT29 cell proliferation inhibitory activity

| Compound number | IC₅₀ (nM) |
|---|---|
| 7-5 | 1.4 |
| 7-9 | 14.9 |
| 7-36 | 14.7 |
| 7-37 | 16.2 |
| 7-38 | 15.4 |
| 7-41 | 9.8 |

TABLE 4-2
HT29 cell proliferation inhibitory activity

| Compound number | IC₅₀ (nM) |
|---|---|
| 7-3 | 10.2 |
| 7-39 | 9.7 |
| 7-42 | 99.7 |
| 7-43 | 10.2 |

The detection results showed that the compounds of the present invention had relatively significant inhibitory effects on the proliferation of HT29 colon cancer cells.

### 3. Inhibitory Effect of Compounds on HCC827 Cell Proliferation

(1) Cell plating: First, tumor cells HCC827 were cultured in the corresponding culture medium, the cells were digested with trypsin, and the cells were resuspended after centrifugation and counted. The cells were adjusted to an appropriate concentration for plating. The source of tumor cells is shown in Table 5.

TABLE 5
Source of tumor cells

| Cell name | Tumor type | Source |
|---|---|---|
| HCC827 | Non-small cell | ATCC |
| | lung cancer | |

Co-incubation of the compound of the present invention and tumor cells: After the cells adhered, the culture medium was removed from the cell culture plates, and the diluted biologically active molecule (the compound of the present invention) was added to the wells of the above plate, and incubated for 72 hours.

Detection of in vitro cell activity: After the incubation was completed, 50 μL of Cell Counting-Lite™ 2.0 reagent (Vazyme) was added to each well, shaken and mixed well in the dark to perform the reaction for 10 minutes, then the detection was carried out, and a microplate reader (manufacturer: BMG, model: PHERAStar-FS) was used for reading. The background RLU was obtained using Cell Counting-Lite™ reagent in cell-free culture medium, and the solvent RLU was obtained using Cell Counting-Lite™ reagent in cell-containing culture medium. Cell inhibition rate=1−(sample RLU−background RLU)/(solvent RLU−background RLU)×100%, and the half maximal inhibitory concentration (IC₅₀) of the compound was calculated according to the four-parameter model fitting curve. RLU: relative light unit. The detection results are shown in Table 6-1 and Table 6-2.

### (2) Data Results

TABLE 6

| Inhibition activity of compounds on | |
|---|---|
| proliferation of HCC827 cells | |
| Compound number | IC₅₀ (nM) |
| 7-5 | 13.1 |
| 7-9 | 115.9 |
| 7-36 | 126.1 |
| 7-37 | 124.0 |
| 7-38 | 138.8 |
| 7-41 | 225.1 |

The detection results showed that the compounds of the present invention had inhibitory effects on the proliferation of HCC827 human non-small cell lung cancer cells.

### 4. Inhibitory Effect of Compounds on Proliferation of HCC1954 Cells

(1) Cell plating: First, tumor cells HCC1954 were cultured in the corresponding culture medium, the cells were digested with trypsin, and the cells were resuspended after centrifugation and counted. The cells were adjusted to an appropriate concentration for plating. The source of tumor cells is shown in Table 7.

TABLE 5
Source of tumor cells

| Cell name | Tumor type | Source |
|---|---|---|
| HCC1954 | Human | Kangnuotai |
| | breast | Biotechnology |
| | cancer | Co., Ltd. |

Co-incubation of the compound of the present invention and tumor cells: After the cells adhered, the culture medium was removed from the cell culture plates, and the diluted biologically active molecule (the compound of the present invention) was added to the wells of the above plate, and incubated for 72 hours.

Detection of in vitro cell activity: After the incubation was completed, 50 μL of Cell Counting-Lite™ 2.0 reagent (Vazyme) was added to each well, shaken and mixed well in the dark to perform the reaction for 10 minutes, then the detection was carried out, and a microplate reader (manufacturer: BMG, model: PHERAStar-FS) was used for reading. The background RLU was obtained using Cell Counting-Lite™ reagent in cell-free culture medium, and the solvent RLU was obtained using Cell Counting-Lite™ reagent in cell-containing culture medium. Cell inhibition rate=1−(sample RLU−background RLU)/(solvent RLU−background RLU)×100%, and the half maximal inhibitory concentration (IC₅₀) of the compound was calculated according to the four-parameter model fitting curve. RLU: relative light unit. The detection results are shown in Table 8-1 and Table 8-2.

### (2) Data Results

TABLE 8-1

| Inhibition activity of compounds on | |
|---|---|
| proliferation of HCC1954 cells | |
| Compound number | IC₅₀ (nM) |
| 7-5 | 25.9 |
| 7-9 | 124.6 |
| 7-36 | 143.6 |
| 7-37 | 147.7 |
| 7-38 | 694.8 |
| 7-41 | 258.9 |

TABLE 8-2

| Inhibitory activity of compounds on | |
|---|---|
| proliferation of HCC1954 cells | |
| Compound number | IC₅₀ (nM) |
| 7-3 | 207.5 |
| 7-29 | 153.9 |
| 7-39 | 139.9 |
| 7-40 | 127.7 |

The detection results showed that the compounds of the present invention had inhibitory effects on the proliferation of HCC1954 human non-small cell lung cancer cells.

### 5. Inhibitory Effect of Compounds on Proliferation of NCI-H1975 Cells

(1) Cell plating: First, tumor cells NCI-H1975 were cultured in the corresponding culture medium, the cells were digested with trypsin, and the cells were resuspended after centrifugation and counted. The cells were adjusted to an appropriate concentration for plating. The source of tumor cells is shown in Table 9.

TABLE 5
Source of tumor cells

| Cell name | Tumor type | Source |
|---|---|---|
| NCI-H1975 | Human | Nanjing Kebai |
| | non-small | Biotechnology |
| | cell lung | Co., Ltd. |
| | cancer | |

Co-incubation of the compound of the present invention and tumor cells: After the cells adhered, the culture medium was removed from the cell culture plates, and the diluted biologically active molecule (the compound of the present invention) was added to the wells of the above plate, and incubated for 72 hours.

Detection of in vitro cell activity: After the incubation was completed, 50 μL of Cell Counting-Lite™ 2.0 reagent (Vazyme) was added to each well, shaken and mixed well in the dark to perform the reaction for 10 minutes, then the detection was carried out, and a microplate reader (manufacturer: BMG, model: PHERAStar-FS) was used for reading. The background RLU was obtained using Cell Counting-Lite™ reagent in cell-free culture medium, and the solvent RLU was obtained using Cell Counting-Lite™ reagent in cell-containing culture medium. Cell inhibition rate=1−(sample RLU−background RLU)/(solvent RLU−background RLU)×100%, and the half maximal inhibitory concentration (IC₅₀) of the compound was calculated according to the four-parameter model fitting curve. RLU: relative light unit. The detection results are shown in Table 10.

### (2) Data Results

TABLE 10

| Inhibitory activity of compounds on | |
|---|---|
| proliferation of NCI-H1975 cells | |
| Compound number | IC₅₀ (nM) |
| 7-5 | 12.3 |
| 7-3 | 124.4 |
| 7-9 | 107.3 |
| 7-36 | 113.5 |
| 7-37 | 101.1 |
| 7-38 | 105.4 |
| 7-39 | 12.3 |
| 7-41 | 69.9 |

The detection results showed that the compounds of the present invention had inhibitory effects on the proliferation of NCI-H1975 human non-small cell lung cancer cells.

Although the specific embodiments of the present invention have been described in detail, those skilled in the art will understand that various modifications and substitutions can be made to those details based on all the teachings disclosed, and these changes are within the scope of protection of the present invention. The full scope of the present invention is given by the appended claims and any equivalents thereof.

What is claimed is:

1. A compound or a pharmaceutically acceptable salt, ester, stereoisomer, polymorph, solvate, N-oxide, isotopically-labeled substance, metabolite or prodrug thereof, wherein the compound has the following structure: 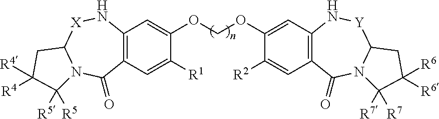
wherein, R¹ and R² are each independently selected from the group consisting of hydrogen, —CN, halogen, —OR^{a}, —NH₂, —NH(C₁₋₆ alkyl), —N(C₁₋₆ alkyl)₂, C₁₋₆ alkyl, C₂₋₆ alkenyl, C₂₋₆ alkynyl, C₃₋₆ cycloalkyl, 3- to 6-membered heterocyclyl, C₆₋₁₀ aryl and 5- to 10-membered heteroaryl; the alkyl, alkenyl, alkynyl, cycloalkyl, heterocyclyl, aryl and heteroaryl are optionally substituted with one or more substituents selected from the group consisting of —CN, halogen, —OH, —NH₂, C₁₋₆ alkyl, C₁₋₆ alkoxy, C₂₋₆ alkenyl, C₂₋₆ alkynyl, C₃₋₆ cycloalkyl, 3- to 6-membered heterocyclyl, C₆₋₁₀ aryl and 5- to 10-membered heteroaryl;
R⁴ and R^{4′} are each independently selected from the group consisting of hydrogen, halogen, —OH, —NH₂, C₁₋₆ alkyl, halogenated C₁₋₆ alkyl and C₁₋₆ alkoxy, both R⁵ and R^{5′} are hydrogen; or, R⁴ and R^{4′}, together with the carbon atom connecting R⁴ and R^{4′}, form a carbon-carbon double bond or a 3- to 6-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3- to 6-membered carbocyclic ring, and R^{5′} and R^{4′} are each independently selected from the group consisting of hydrogen, halogen, —NH₂, C₁₋₆ alkyl, halogenated C₁₋₆ alkyl and C₁₋₆ alkoxy;
R⁶ is hydrogen, C₁₋₆ alkyl or 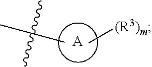
R^{6′}, R⁷ and R^{7′} are all hydrogen; or, R^{6′} and R⁷ are connected to form a bond, and R^{7′} is hydrogen;
R^{a} is selected from the group consisting of H, C₁₋₆ alkyl or C₃₋₆ cycloalkyl;
Ring A is selected from the group consisting of 3- to 6-membered cycloalkyl, C₆₋₁₀ aryl, 5- to 10-membered heteroaryl, 3- to 6-membered heterocyclyl;
R³ is selected from the group consisting of hydrogen, —CN, halogen, —OR^{a}, —NH₂, —NH(C₁₋₆ alkyl), —N(C₁₋₆ alkyl)₂, C₁₋₆ alkyl, C₃₋₆ cycloalkyl, 3- to 6-membered heterocyclyl, C₆₋₁₀ aryl and 5- to 10-membered heteroaryl; the alkyl, alkenyl, alkynyl, cycloalkyl, heterocyclyl, aryl and heteroaryl are optionally substituted with one or more substituents selected from the group consisting of —CN, halogen, —OH, —NH₂, C₁₋₆ alkyl, C₁₋₆ alkoxy, C₃₋₆ cycloalkyl, 3- to 6-membered heterocyclyl, C₆₋₁₀ aryl and 5- to 10-membered heteroaryl;
X and Y are each independently selected from the group consisting of —CH₂—, —CD₂-, 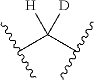 [SMILES: *C(*)[2H]]
and —C(O)—;
m is selected from 1 to 5, such as 1, 2, 3, 4 or 5;
n is selected from 1 to 10, such as 1, 2, 3, 4, 5, 6, 7, 8, 9 or 10;
when n is 5 and Ring A is phenyl, R³ is not methoxy.

2. The compound or pharmaceutically acceptable salt, ester, stereoisomer, polymorph, solvate, N-oxide, isotopically-labeled substance, metabolite or prodrug thereof according to claim 1, wherein
R¹ is selected from the group consisting of hydrogen, —CN, halogen, —OR^{a} and C₁₋₆ alkyl, wherein R^{a} is selected from the group consisting of C₁₋₆ alkyl, deuterated C₁₋₆ alkyl and C₃₋₆ cycloalkyl;
preferably, R¹ is selected from the group consisting of —OR^{a}, wherein R^{a} is selected from the group consisting of C₁₋₆ alkyl, deuterated C₁₋₆ alkyl and C₃₋₆ cycloalkyl;
preferably, R¹ is selected from the group consisting of methoxy, trideuterated methoxy and —O-cyclopropyl.

3. The compound or pharmaceutically acceptable salt, ester, stereoisomer, polymorph, solvate, N-oxide, isotopically-labeled substance, metabolite or prodrug thereof according to claim 1 or 2, wherein
R² is selected from the group consisting of hydrogen, —CN, halogen, —OR^{a} and C₁₋₆ alkyl, wherein R^{a} is C₁₋₆ alkyl or C₃₋₆ cycloalkyl;
preferably, R² is —OR^{a}, wherein R^{a} is C₁₋₆ alkyl;
preferably, R² is selected from the group consisting of methoxy.

4. The compound or pharmaceutically acceptable salt, ester, stereoisomer, polymorph, solvate, N-oxide, isotopically-labeled substance, metabolite or prodrug thereof according to any one of claims 1 to 3, wherein
R⁴ is hydrogen or halogen, R⁴ is selected from the group consisting of hydrogen, halogen, —OH, —NH₂, C₁₋₆ alkyl, halogenated C₁₋₆ alkyl and C₁₋₆ alkoxy, both R⁵ and R^{5′} are hydrogen; or, R⁴ and R^{4′}, together with the carbon atom connecting R⁴ and R^{4′}, form a carbon-carbon double bond or a 3- to 6-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3- to 6-membered carbocyclic ring, and both R^{5′} and R^{4′} are hydrogen;
preferably, R⁴ is hydrogen or fluorine, R^{4′} is selected from the group consisting of hydrogen, fluorine, —OH, —NH₂, methyl, trifluoromethyl and methoxy, both R⁵ and R^{5′} are hydrogen; or, R⁴ and R^{4′}, together with the carbon atom connecting R⁴ and R^{4′}, form a carbon-carbon double bond or a 3- to 6-membered carbocyclic ring, and both R⁵ and R^{5′} are hydrogen; or, R⁴ and R⁵ are connected to form a bond, or R⁴ and R⁵, together with the carbon atom connecting R⁴ and R⁵, form a 3-membered carbocyclic ring, and both R^{5′} and R^{4′} are hydrogen;
preferably, R⁴ is hydrogen or fluorine, R^{4′} is selected from the group consisting of hydrogen, fluorine, —OH, —NH₂, methyl, trifluoromethyl and methoxy, and both R⁵ and R^{5′} are hydrogen; or, R⁴ and R^{4′}, together with the carbon atom connecting R⁴ and R^{4′}, form a carbon-carbon double bond, and both R⁵ and R^{5′} are hydrogen; or, R⁴ and R⁵ are connected to form a bond, or R⁴ and R⁵, together with the carbon atom connecting R⁴ and R⁵, form a 3-membered carbocyclic ring, and both R^{5′} and R^{4′} are hydrogen;
preferably, R⁴ and R^{4′}, together with the carbon atom connecting R⁴ and R^{4′}, form a 3-membered carbocyclic ring, and both R⁵ and R^{5′} are hydrogen.

5. The compound or pharmaceutically acceptable salt, ester, stereoisomer, polymorph, solvate, N-oxide, isotopically-labeled substance, metabolite or prodrug thereof according to any one of claims 1 to 4, wherein
R⁶ is 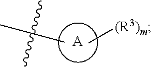
Ring A is selected from the group consisting of 3- to 6-membered cycloalkyl, C₆₋₁₀ aryl or 5- to 10-membered heteroaryl;
R³ is selected from the group consisting of hydrogen, —CN, halogen, —OR^{a}, —NH₂, —NH(C₁₋₆ alkyl), —N(C₁₋₆ alkyl)₂, C₁₋₆ alkyl, C₃₋₆ cycloalkyl, 3- to 6-membered heterocyclyl, C₆₋₁₀ aryl and 5- to 10-membered heteroaryl; the alkyl, alkenyl, alkynyl, cycloalkyl, heterocyclyl, aryl and heteroaryl are optionally substituted with one or more substituents selected from the group consisting of —CN, halogen, —OH, —NH₂, C₁₋₆ alkyl, C₁₋₆ alkoxy, C₃₋₆ cycloalkyl, 3- to 6-membered heterocyclyl, C₆₋₁₀ aryl and 5- to 10-membered heteroaryl;
R^{a} is selected from the group consisting of H and C₁₋₆ alkyl;
m is selected from 1, 2, 3, 4 and 5;
preferably, R⁶ is 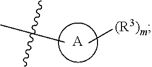
Ring A is selected from the group consisting of cyclopropyl, phenyl, furanyl, thienyl, pyrimidinyl and pyridinyl;
R³ is selected from the group consisting of hydrogen, fluorine, —OH, methoxy, —NH₂ and hydroxymethyl;
m is 1 or 2;
preferably, R⁶ is 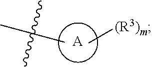
Ring A is phenyl;
R³ is selected from the group consisting of hydrogen, fluorine, —OH, methoxy, —NH₂ and hydroxymethyl;
m is 1 or 2.

6. The compound or pharmaceutically acceptable salt, ester, stereoisomer, polymorph, solvate, N-oxide, isotopically-labeled substance, metabolite or prodrug thereof according to any one of claims 1 to 5, wherein
X is —CH₂— or —CD₂-; optionally, Y is selected from the group consisting of —CH₂—, —CD₂-, 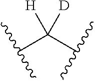 [SMILES: *C(*)[2H]]
and —C(O)—;
preferably, X is —CH₂— and Y is —CH₂—; X is —CH₂— and Y is —CD₂- or —C(O)—; or X is —CD₂- and Y is —CH₂—;
preferably, X is —CH₂— and Y is —CH₂—.

7. The compound or pharmaceutically acceptable salt, ester, stereoisomer, polymorph, solvate, N-oxide, isotopically-labeled substance, metabolite or prodrug thereof according to any one of claims 1 to 6, wherein the compound has a structure represented by the following Formula (I-1) or Formula (I-2): 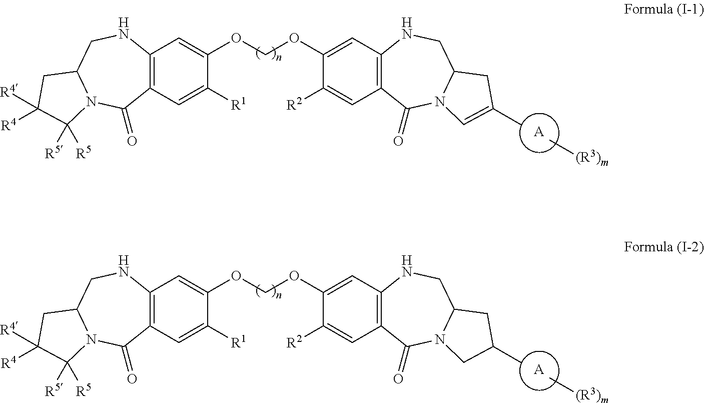
wherein, R¹, R², R³, R⁴, R^{4′}, R⁵, R^{5′}, Ring A, m and n are as described in any one of claims 1 to 6.

8. The compound or pharmaceutically acceptable salt, ester, stereoisomer, polymorph, solvate, N-oxide, isotopically-labeled substance, metabolite or prodrug thereof according to any one of claims 1 to 7, wherein the compound has a structure represented by the following Formula (II-1) or Formula (II-2): 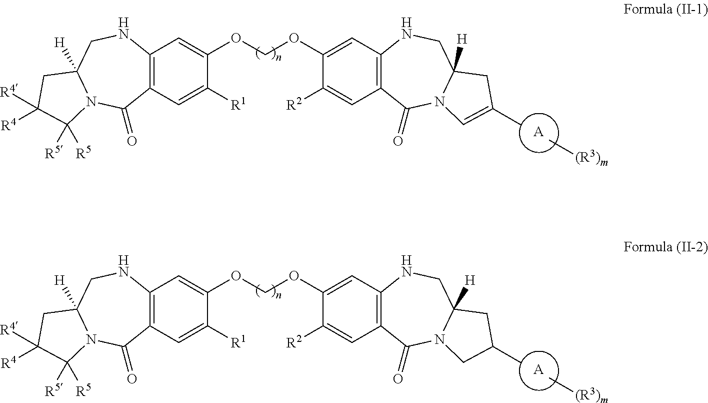
wherein, R¹, R², R³, R⁴, R^{4′}, R⁵, R^{5′}, Ring A, m and n are as described in any one of claims 1 to 6.

9. The compound or pharmaceutically acceptable salt, ester, stereoisomer, polymorph, solvate, N-oxide, isotopically-labeled substance, metabolite or prodrug thereof according to any one of claims 1 to 8, wherein the compound is selected from the group consisting of the following structures:

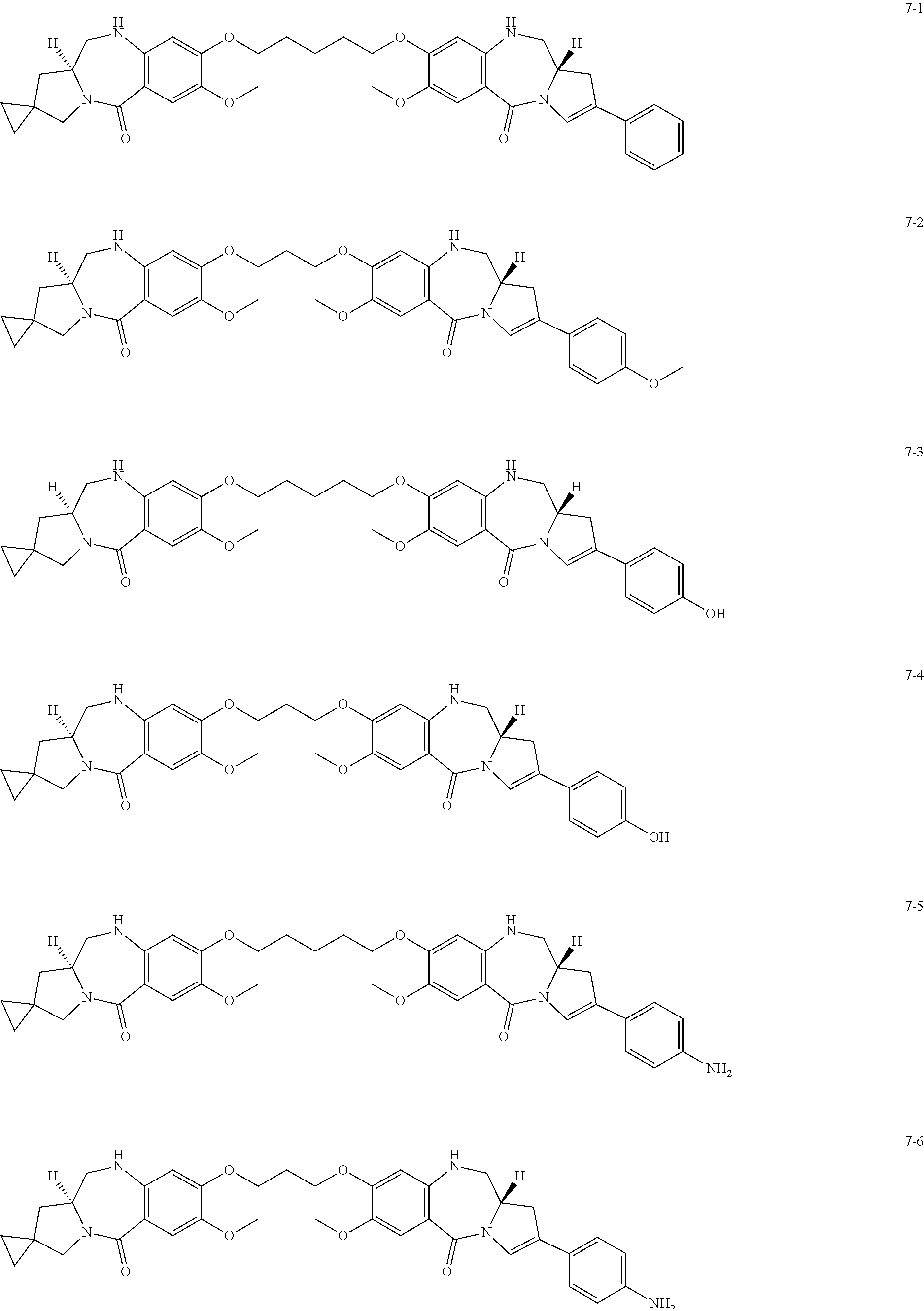

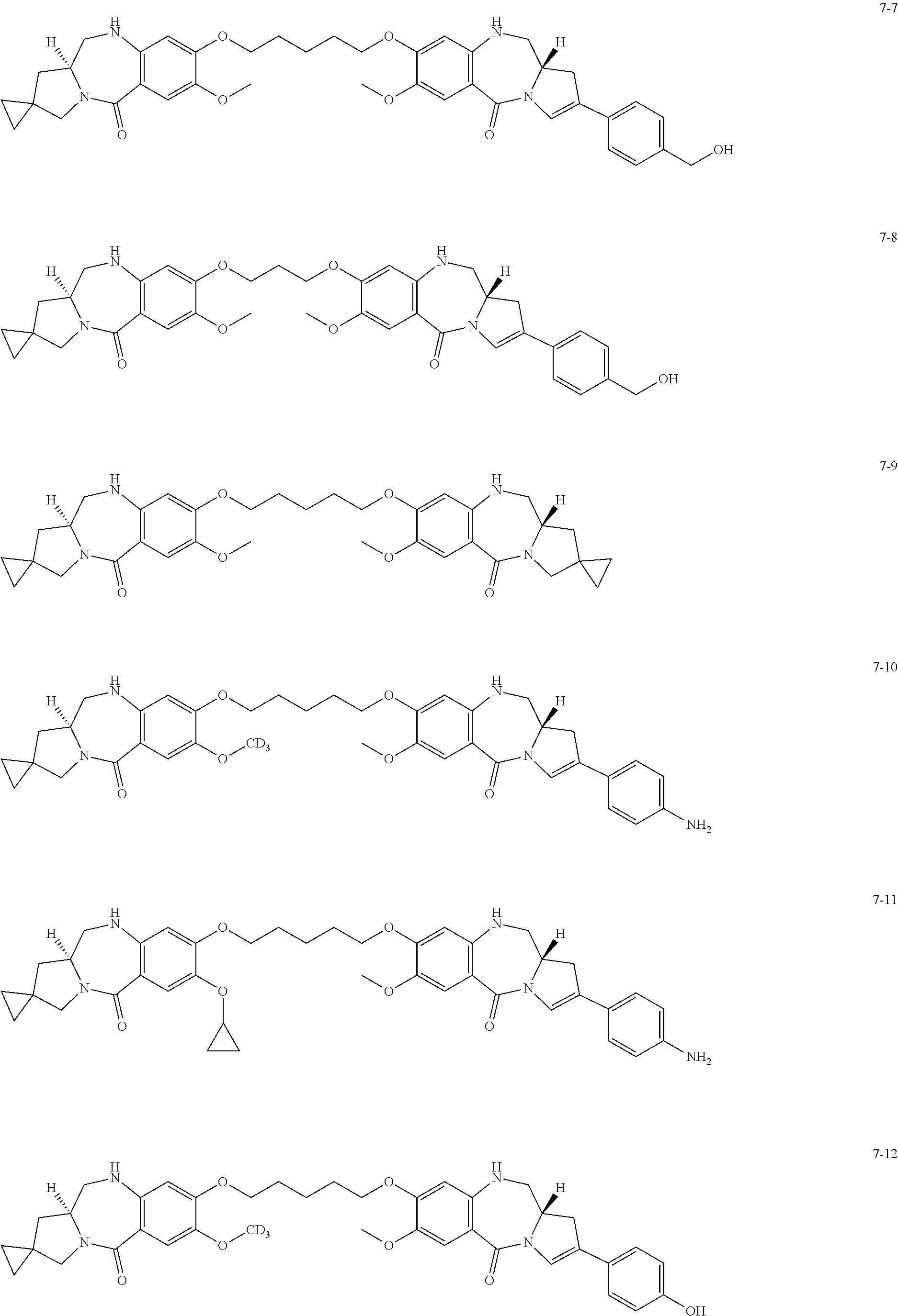

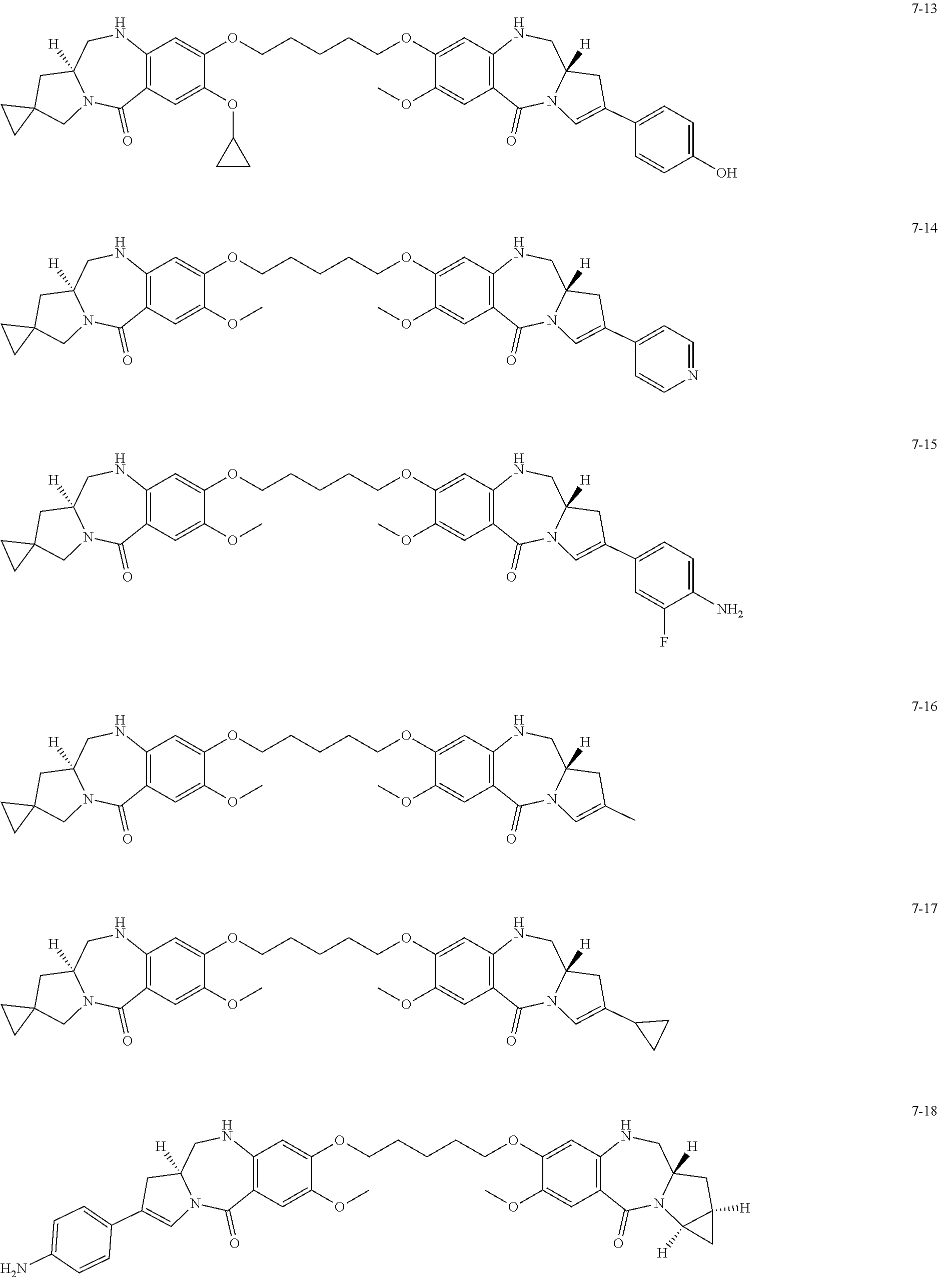

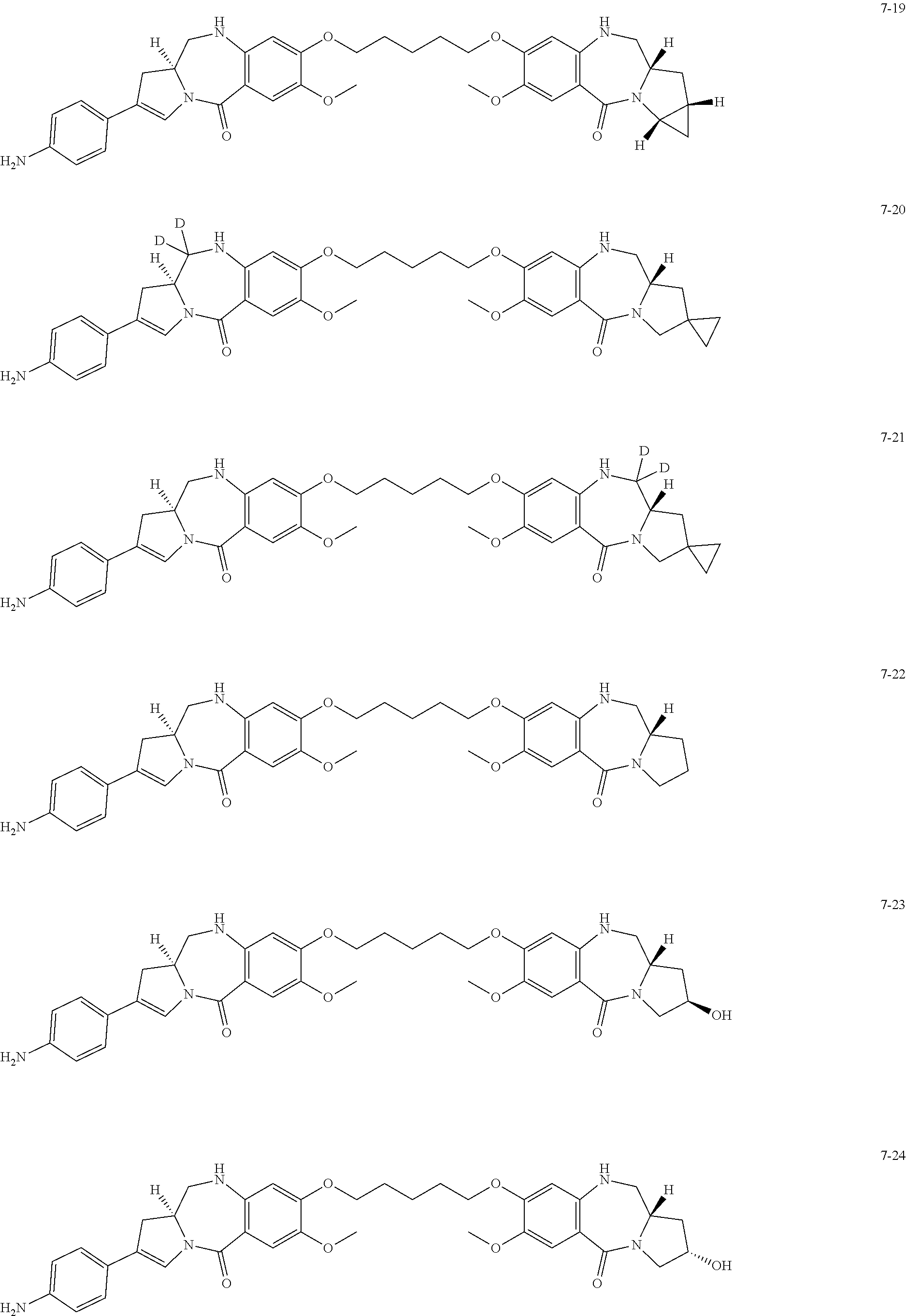

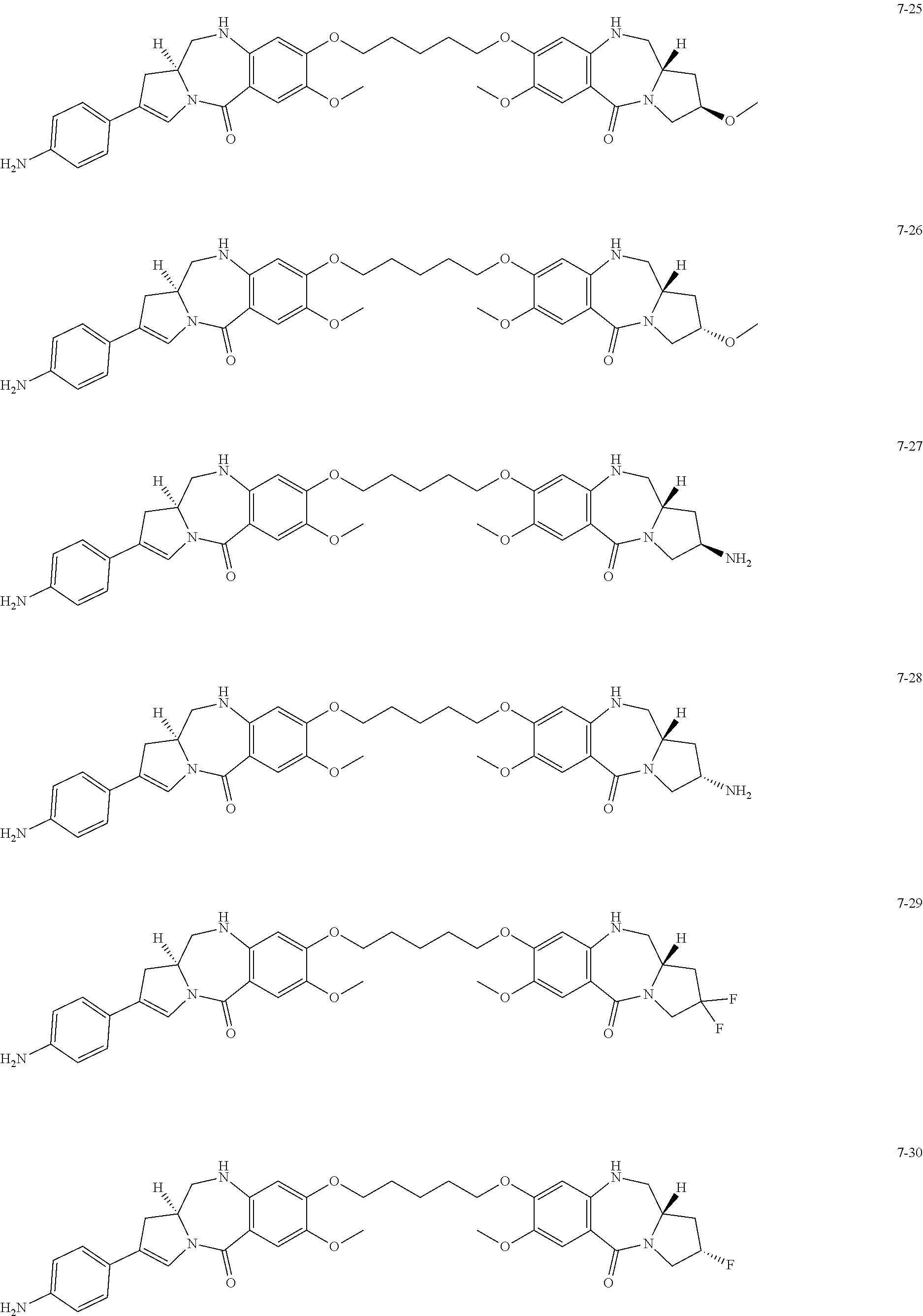

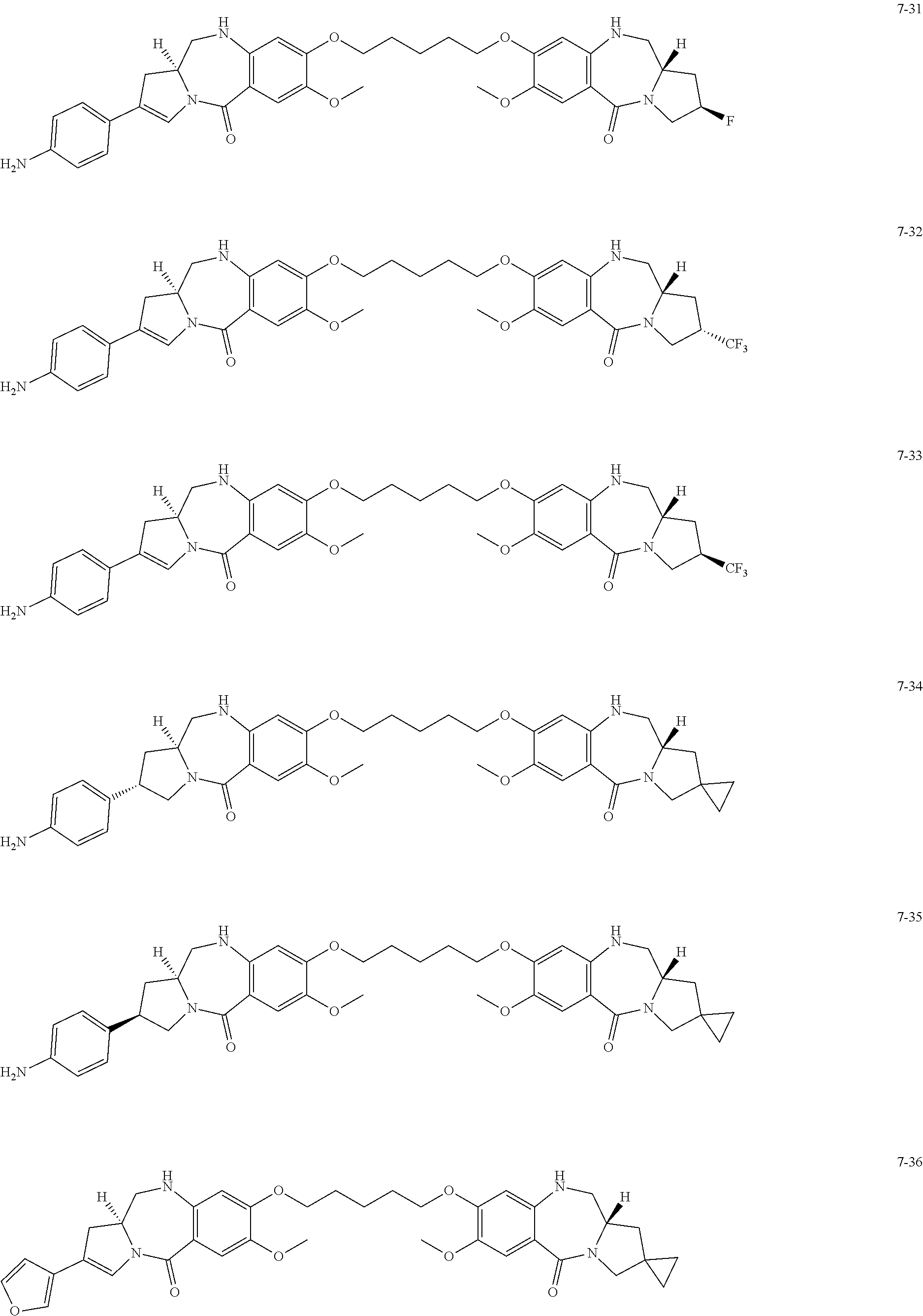

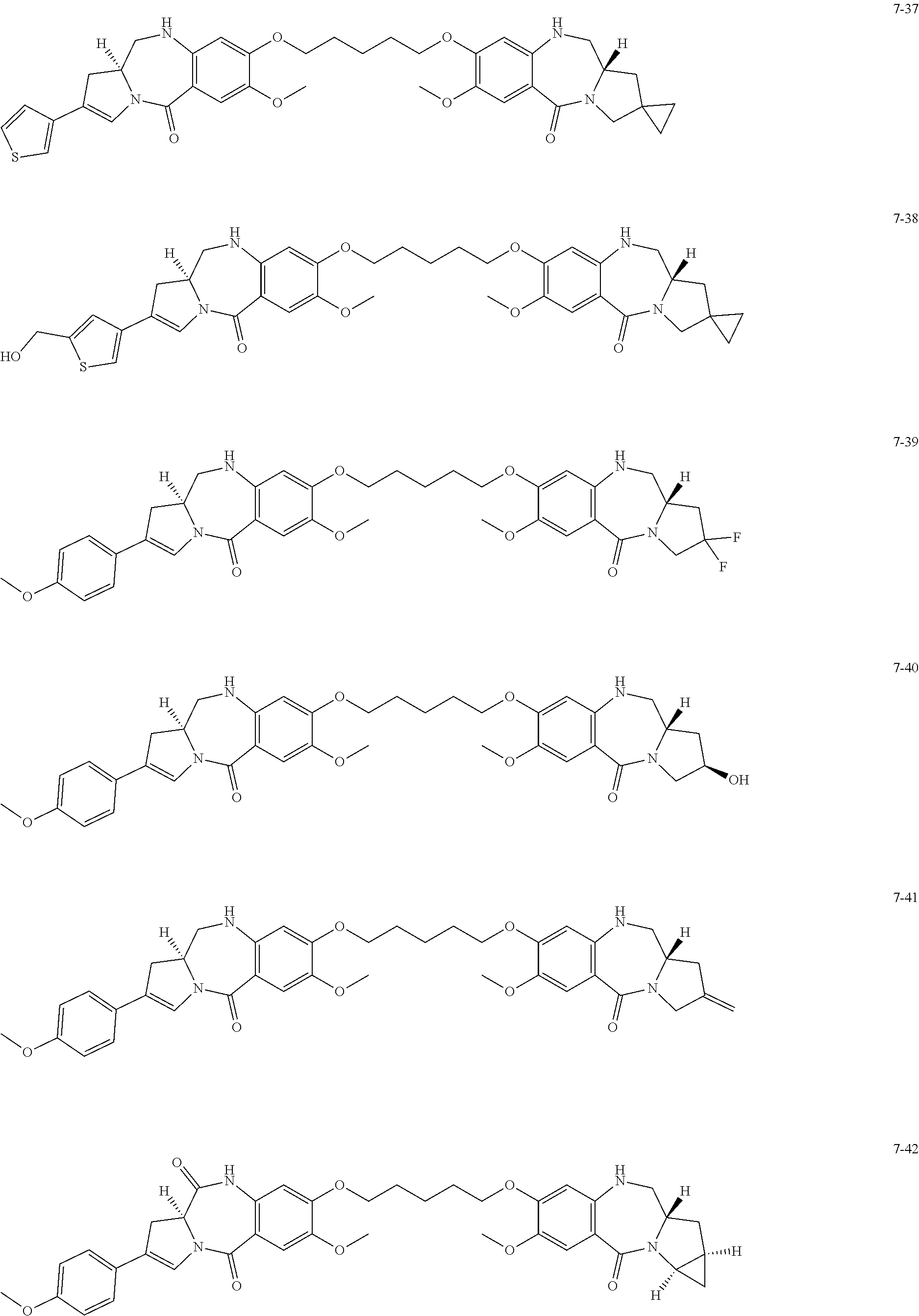

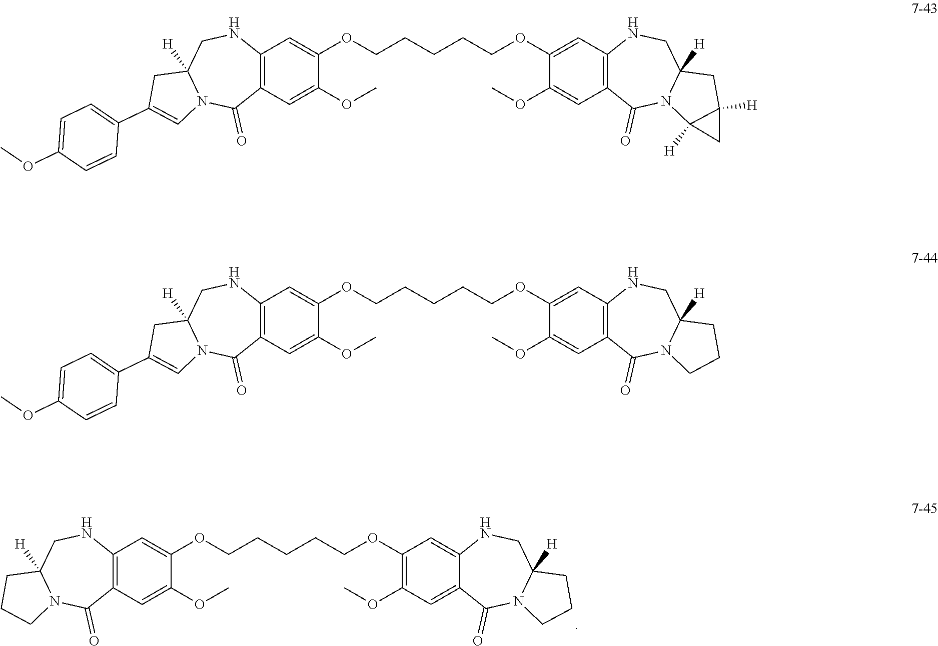 [SMILES: COc1ccc(C2=CN3C(=O)c4cc(OC)c(OCCCCCOc5cc6c(cc5OC)C(=O)N5[C@H](CN6)C[C@H]6C[C@H]65)cc4NC[C@@H]3C2)cc1 ; COc1ccc(C2=CN3C(=O)c4cc(OC)c(OCCCCCOc5cc6c(cc5OC)C(=O)N5CCC[C@H]5CN6)cc4NC[C@@H]3C2)cc1 ; COc1cc2c(cc1OCCCCCOc1cc3c(cc1OC)C(=O)N1CCC[C@H]1CN3)NC[C@@H]1CCCN1C2=O]

10. An antibody-drug conjugate, which comprises a moiety of the compound or pharmaceutically acceptable salt, ester, stereoisomer, polymorph, solvate, N-oxide, isotopically-labeled substance, metabolite or prodrug thereof according to any one of claims 1 to 9.

11. A pharmaceutical composition, which comprises the compound or pharmaceutically acceptable salt, ester, stereoisomer, polymorph, solvate, N-oxide, isotopically-labeled substance, metabolite or prodrug thereof according to any one of claims 1 to 9, or the antibody-drug conjugate according to claim 10, and one or more pharmaceutical excipients.

12. Use of the compound or pharmaceutically acceptable salt, ester, stereoisomer, polymorph, solvate, N-oxide, isotopically-labeled substance, metabolite or prodrug thereof according to any one of claims 1 to 9, the antibody-drug conjugate according to claim 10, or the pharmaceutical composition according to claim 11, in the manufacture of a medicament for treating a cancer or tumor;
optionally, the cancer or tumor is a solid tumor or a hematological malignancy, such as gastric cancer, breast cancer, lung cancer (e.g., non-small cell lung cancer, specifically lung adenocarcinoma) or urothelial carcinoma.

13. The compound or pharmaceutically acceptable salt, ester, stereoisomer, polymorph, solvate, N-oxide, isotopically-labeled substance, metabolite or prodrug thereof according to any one of claims 1 to 9, the antibody-drug conjugate according to claim 10, or the pharmaceutical composition according to claim 11, for use in treating a cancer or tumor;
optionally, the cancer or tumor is a solid tumor or a hematological malignancy, such as gastric cancer, breast cancer, lung cancer (e.g., non-small cell lung cancer, specifically lung adenocarcinoma) or urothelial carcinoma.

14. A method for treating a cancer or tumor, comprising administering to an individual in need thereof a therapeutically effective amount of the compound or pharmaceutically acceptable salt, ester, stereoisomer, polymorph, solvate, N-oxide, isotopically-labeled substance, metabolite or prodrug thereof according to any one of claims 1 to 9, the antibody-drug conjugate according to claim 10, or the pharmaceutical composition according to claim 11;
optionally, the cancer or tumor is a solid tumor or a hematological malignancy, such as gastric cancer, breast cancer, lung cancer (e.g., non-small cell lung cancer, specifically lung adenocarcinoma) or urothelial carcinoma.
